(19) United States
(12) Patent Application Publication
(10) Pub. No.: US 2003/0165852 A1
(43) Pub. Date: Sep. 4, 2003

(54) Methods and reagents for identifying rare fetal cells in the maternal circulation

(76) Inventors: Paula A. Schueler, Woodinville, WA (US); Hongxia Xu, Castro Valley, CA (US); Lisa Foltz, Indianapolis, IN (US); Xingyong Wu, Pleasanton, CA (US); Yehsiung Sha, Castro Valley, CA (US); Alexandra Nagy, Alameda, CA (US); Walter C. Mahoney, Woodinville, WA (US)

Correspondence Address: Kenneth J. Waite, Roche Diagnostics Corporation, 9115 Hague Road, Bldg D, Indianapolis, IN, 46250-0457, US

(21) Appl. No.: 10/000,897

(22) Filed: Nov. 15, 2001

Related U.S. Application Data
(60) Provisional application No. 60/248,882, filed on Nov. 15, 2000.

(51) Int. Cl. C12Q 1/68

(52) U.S. Cl. 435/6

(57) ABSTRACT

This invention provides methods and compositions useful for identifying and diagnosing rare fetal cells in a mixed cell population such as a maternal blood sample. The methods entail the use of specific nucleic acid probes that hybridize to fetal cell associated RNAs to identify the rare fetal cells or antibodies that bind to polypeptides encoded by the fetal cell associated RNAs for fetal cell detection. The cells detected by the methods of the present invention are useful for diagnosing the fetal cells for a genetic trait of interest, such as trisomy 21. Novel methods for simultaneous screening for fetal cells and diagnosing the fetal cells are also provided. Compositions comprising the fetal cell associated nucleic acids of the invention and their encoded proteins are also provided. The present invention further provides kits useful for practicing the present methods.

The present application claims priority of U.S. provisional application No. 60/248,882 under 35 U.S.C. §119(e), which application is incorporated by reference herein in its entirety.

### 1. FIELD OF THE INVENTION

This invention relates generally to the fields of cell purification, cell identification, and prenatal genetic analysis. More particularly, the invention provides methods and compositions for identifying individual cells of fetal origin in samples of maternal blood. The methods encompass the use of specific nucleic acid probes to identify the rare fetal cells in the maternal blood sample and optionally further diagnosing the detected fetal cells for a genetic trait of interest. Compositions comprising the nucleic acid probes and kits useful in the present methods are also provided.

### 2. BACKGROUND OF THE INVENTION

Amniocentesis and chorionic villus sampling are the currently accepted methods for prenatal testing for genetic abnormalities. However, both of these procedures are invasive and are accompanied by a small risk (on the order of 1%) of fetal death. Obtaining and identifying fetal cells in the maternal circulation holds considerable promise for prenatal genetic testing. Particularly advantageous is the fact that the test sample is obtained by a relatively non-invasive procedure that poses essentially no risk to the fetus. In addition, since fetal cells peak in the maternal circulation at about 10-16 weeks of gestation, it is possible to perform the genetic analysis at an early stage in pregnancy. However, fetal cells are extremely rare in maternal blood, on the order of 1 to 50 cells per 10⁷ nucleated blood cells. These low levels of fetal cells make even minimal levels of non-specific binding problematic for affinity separation of fetal cells from maternal cells.

A number of fetal cells are known to make their way into the maternal circulation, including leukocytes, trophoblast cells and nucleated red blood cells. Leukocytes have been generally excluded from consideration as targets for isolation from maternal blood for a number of reasons, including a lack of generic markers for use in isolation as well as the possibility of persistence in maternal blood of fetal leukocytes from previous pregnancies. Trophoblast cells have been considered undesirable due to concerns that these cells may be subject to confined placental mosaicism, rendering them unrepresentative of the fetus. Nucleated fetal erythroid cells, however, are considered an attractive target for prenatal genetic analysis.

The isolation of nucleated fetal erythroid cells from maternal blood has, however, been fraught with difficulty. The low abundance of these cells in maternal blood renders separation extremely difficult, as even extremely low non-specific binding by a separation reagent will result in large numbers of maternal cells if the reagent positively selects for the fetal cells, and unacceptably low yields if the antibody negatively selects for maternal cells.

Also, no fetal blood cell specific markers are known in the art. Enrichment of fetal blood cells has been performed using markers such as fetal hemoglobin (Hemoglobin F or HbF), which is estimated to be present in 0.1% to 0.7% of erythroid cells in normal adult blood. Immature erythroid cells (i.e., cells of the erythroid lineage at the reticulocyte stage and earlier) express markers which have been used to enrich fetal blood cells (e.g., glycophorin A, CD36, and the transferrin receptor, also known as TfR and CD71). However, these markers can also be found on cells in adult blood, and it has also been found that blood samples taken during pregnancy contain relatively high levels of maternal immature erythroid cells.

A variety of methods have been proposed for isolation or enrichment of fetal cells in maternal blood. These methods include centrifugation techniques, immunoaffinity techniques, and fluorescent in situ hybridization (FISH) methods. However, these methods suffer from a number of deficiencies.

Centrifugation methods generally rely on density gradients for separation of nucleated from non-nucleated cells and frequently include a lysis step to eliminate erythrocytes. See, for example, U.S. Pat. Nos. 5,432,054, and 5,646,004, International Patent Application No. WO 95/09245 and Rao et al. (1994, Ann. NY Acad. Sci. 731:142-143). However, these techniques co-enrich large numbers of maternal nucleated erythroid cells, and so do not provide the level of enrichment required for reproducible genetic screening of fetal cells.

Immunoaffinity approaches have been described using a variety of different antibodies. However, most approaches rely on the use of antibodies directed to markers in the erythroid pathway. For example, Bianchi et al. (1993, Prenatal Diag. 13:293-300) describes a method utilizing CD71 (transferrin receptor) CD36 (thrombospondin receptor) and/or glycophorin A antibodies for flow sorting to enrich ‘fetal’ cells from maternal blood. The use of antibodies to erythroid cell markers such as CD71, CD36 and glycophorin co-enriches maternal erythroid cells, which substantially outnumber fetal erythroid cells in maternal blood samples.

Fluorescent in situ hybridization methods have been used to sort cells which express particular RNAs from maternal blood samples. These methods suffer from the same problems as immunoaffinity methods, due to the lack of fetal cell specific probes. WO 96//17085 teaches the use of probes specific for HLA-G, a non-classical Class I MHC molecule which is an oncofetal marker found on extravillous cytotrophoblast cells, for use in sorting HLA-G expressing cells from samples, such a maternal blood.

Fetal cells present in the maternal circulation are at various stages of development. As with other cell types, expression patterns of cellular markers change as a given cell proceeds down a developmental pathway. Reagents for identifying fetal cells must accommodate such variations. Accordingly, there is a need in the art for new reagents and methods for separation and identification of fetal cells in maternal blood.

Citation or identification of any reference herein shall not be construed as an admission that such reference is available as prior art to the present invention.

### 3. SUMMARY OF THE INVENTION

The present invention provides methods for detecting a fetal cell in a maternal blood sample, comprising the steps of: (a) contacting said maternal blood sample with a first probe comprising a nucleotide sequence corresponding to SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, which first probe selectively or specifically hybridizes to mRNA in fetal cells if present in the maternal blood sample; and (b) identifying whether or not said maternal blood sample comprises a cell that comprises mRNA that detectably hybridizes to the first probe, thereby detecting whether or not said maternal blood sample contains a fetal cell.

The present invention further provides methods for detecting a fetal cell in a maternal blood sample, comprising the steps of: (a) contacting said maternal blood sample with a first probe comprising a nucleotide sequence having at least 90% sequence identity to at least 20 consecutive nucleotides of SEQ ID NO:, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, which first probe selectively or specifically hybridizes to fetal cells if present in the maternal blood sample; and (b) identifying whether or not said maternal blood sample comprises a cell that comprises mRNA that detectably hybridizes to the first probe, thereby detecting whether or not said maternal blood sample contains a fetal cell.

The present invention yet further provides methods for detecting a fetal cell in a maternal blood sample, comprising the steps of: (a) contacting said maternal blood sample with a first probe comprising a nucleotide sequence having at least 80% sequence identity to at least 40 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, which first probe selectively or specifically hybridizes to fetal cells if present in the maternal blood sample; and (b) identifying whether or not said maternal blood sample comprises a cell that comprises mRNA that detectably hybridizes to the first probe, thereby detecting whether or not said maternal blood sample contains a fetal cell.

The present invention yet further provides methods for detecting a fetal cell in a maternal blood sample, comprising the steps of: (a) performing differential expression analysis on RNA or cDNA obtained from fetal liver myeloid cells relative to RNA or cDNA obtained from mature myeloid cells; (b) identifying an RNA or cDNA species that is selectively or specifically expressed in the fetal liver myeloid cells, thereby identifying an RNA or cDNA species that is useful as a probe for fetal cells in the maternal circulation; (c) contacting the maternal blood sample with a probe comprising a nucleotide sequence corresponding to all or a portion of the RNA or cDNA of step (b); and (d) identifying whether or not said maternal blood sample comprises a cell that comprises mRNA that detectably hybridizes to the first probe, thereby detecting whether or not said maternal blood sample contains a fetal cell.

The present invention further provides methods for diagnosing an abnormality in a fetal cell, comprising the steps of: (a) contacting a maternal blood sample with a first probe comprising a nucleotide sequence corresponding to SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, which first probe selectively or specifically hybridizes to mRNA in the fetal cell if present in the maternal blood sample; (b) identifying whether or not said maternal blood sample comprises a cell that comprises mRNA that detectably hybridizes to the first probe, thereby detecting whether or not said maternal blood sample contains a fetal cell; and (c) if the maternal blood sample comprises a fetal cell, determining whether the abnormality exists in said fetal cell, thereby diagnosing the abnormality. The fetal cell detection and diagnostic steps can be performed concurrently or successively (in either order).

The present invention further provides methods for diagnosing an abnormality in a fetal cell, comprising the steps of: (a) contacting a maternal blood sample comprising said fetal cell with a first probe comprising a nucleotide sequence having at least 90% sequence identity to at least 20 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, which first probe selectively or specifically hybridizes to mRNA in the fetal cell if present in the maternal blood sample; (b) identifying whether or not said maternal blood sample comprises a cell that comprises mRNA that detectably hybridizes to the first probe, thereby detecting whether or not said maternal blood sample contains a fetal cell, and (c) if the maternal blood sample contains a fetal cell, determining whether the abnormality exists in said fetal cell, thereby diagnosing the abnormality. The fetal cell detection and diagnostic steps can be performed concurrently or successively (in either order).

The present invention further provides methods for diagnosing an abnormality in a fetal cell, comprising the steps of: (a) contacting a maternal blood sample comprising said fetal cell with a first probe comprising a nucleotide sequence having at least 80% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, which first probe selectively or specifically hybridizes to mRNA in the fetal cell if present in the maternal blood sample; (b) identifying whether or not said maternal blood sample comprises a cell that comprises mRNA that detectably hybridizes to the first probe, thereby detecting whether or not said maternal blood sample contains a fetal cell; and (c) if the maternal blood sample contains a fetal cell, determining whether the abnormality exists in said fetal cell, thereby diagnosing the abnormality. The fetal cell detection and diagnostic steps can be performed concurrently or successively (in either order).

The present invention yet further provides methods for diagnosing an abnormality in a fetal cell, comprising the steps of: (a) performing differential expression analysis on RNA or cDNA obtained from fetal liver myeloid cells relative to RNA or cDNA obtained from mature myeloid cells; (b) identifying an RNA or cDNA species that is selectively or specifically expressed in the fetal liver myeloid cells, thereby identifying an RNA or cDNA species that is useful as a probe for fetal cells in the maternal circulation; (c) contacting the maternal blood sample with a probe comprising a nucleotide sequence corresponding to all or a portion of the RNA or cDNA of step (b); (d) identifying whether or not said maternal blood sample comprises a cell that comprises mRNA that detectably hybridizes to the first probe, thereby detecting whether or not said maternal blood sample contains a fetal cell; and (e) if the maternal blood sample comprises a fetal cell, determining whether the abnormality exists in said fetal cell, thereby diagnosing the abnormality.

The fetal cell detection and diagnosis methods of the present invention optionally further comprise contacting the maternal blood sample with a second probe which selectively or specifically hybridizes to fetal cells if present in the maternal blood sample prior to identifying whether a fetal cell is present in the maternal blood sample, and, optionally, detecting a cell in said maternal blood sample which comprises mRNA that hybridizes to the second probe. The second probe is preferably labeled with the same type of label as the first probe. The first and second probes can correspond to the same mRNA or to different mRNAs. In a preferred embodiment, the first probe corresponds to the J42-4d gene (SEQ ID NO:11) and the second probe corresponds to fetal epsilon globin. Such probes are preferably at least 25, most preferably at least 30, and most preferably 150-200 nucleotides in length. The probes are also preferably riboprobe prepared according to the method described in Section 8.1 below.

In certain embodiments of the fetal cell detection and diagnosis methods of the present, the maternal blood sample is immunoenriched for fetal cells prior to contacting the blood sample with the fetal cell specific or selective probe. The maternal blood sample can be positively immunoenriched by (a) contacting the maternal blood sample with an antibody that selectively or specifically binds to fetal cells in the maternal blood sample; and (b) separating cells in the maternal blood sample that bind to the antibody from cells that do not bind to the antibody, thereby immunoenriching the maternal blood sample for fetal cells. Alternatively, the maternal blood sample can be negatively immunoenriched by (a) contacting the maternal blood sample with an antibody that selectively or specifically binds to maternal cells in the maternal blood sample; and (b) separating cells in the maternal blood sample that do not bind to the antibody from cells that bind to the antibody, thereby immunoenriching the maternal blood sample for fetal cells.

The diagnostic methods of the invention can be used to detect chromosomal abnormalities. In certain specific embodiments, the chromosomal abnormalities are aneuploidies, including but not limited to trisomy 13, trisomy 21, or Klinefelter or other sex chromosome syndromes. In other specific embodiments, the chromosomal abnormalities are single gene disorders. The single gene disorder can be a deletion, insertion or substitution disorder. In exemplary embodiments, the single gene disorder is spina bifida, sickle-cell anemia, a thalassemia, Marfan Syndrome, Duchenne Muscular Dystrophy, or cystic fibrosis. In yet other embodiments, the single gene disorders detected by the methods of the present invention are nucleoeotide triplet expansions in one or more genes. Such genes include but are not limited to the Fragile X Syndrome gene, the Friedreich's ataxia gene, the myotonic dystrophy gene, or the Huntington's disease genes. In yet other embodiments, the chromosomal abnormalities are viral sequences, e.g., HIV sequences, inserted in the fetal cell genome.

The present invention yet further provides methods for identifying a nucleic acid useful as a probe for fetal cells in the maternal circulation, comprising the steps of: (a) performing differential expression analysis on RNA or cDNA obtained from fetal liver myeloid cells relative to RNA or cDNA obtained from mature myeloid cells; and (b) identifying an RNA or cDNA species that is selectively or specifically expressed in the fetal liver myeloid cells, wherein the RNA or cDNA species that is selectively or specifically expressed in the fetal liver myeloid cells is useful as a probe for fetal cells in the maternal circulation. In a preferred embodiment, the fetal liver is human fetal liver. In another preferred embodiment, the fetal liver myeloid cells are obtained before 20 weeks of gestation. In preferred modes of the embodiment, the fetal liver myeloid cells are obtained between 10 and 15 weeks of gestation, e.g., at 10, 10.5, 11, 11.5, 12, 12.5, 13, 13.5, 14, 14.5, or 15 weeks of gestation. The mature myeloid cells can be fetal cord blood cells obtained after 20 weeks of gestation, fetal peripheral blood cells obtained after 20 weeks of gestation, fetal liver myeloid cells obtained after about 20 weeks of gestation, adult bone marrow cells or adult peripheral blood cells. In yet another preferred embodiment, the differential expression analysis comprises subtraction suppression hybridization.

The present invention yet further provides kits comprising in one or more containers (a) a first probe comprising a nucleotide sequence corresponding to SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, which first probe selectively or specifically hybridizes to mRNA in fetal cells if present in a maternal blood sample and (b) instructions for diagnostic use or a label indicating regulatory approval for diagnostic use. In other embodiments, the present invention provides kits comprising in one or more containers a first probe comprising a nucleotide sequence having at least 90% sequence identity to at least 20 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, which first probe selectively or specifically hybridizes to fetal cells if present in a maternal blood sample. In yet other embodiments, the present invention provides kits comprising in one or more containers a first probe comprising a nucleotide sequence having at least 80% sequence identity to at least 40 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, which first probe selectively or specifically hybridizes to fetal cells if present in a maternal blood sample. The kits can further comprise one or more antibodies for immunoenriching for fetal cells in a maternal blood sample, for example an antibody that selectively or specifically binds to fetal cells in a maternal blood sample. The kits can also optional comprise a second probe that selectively or specifically hybridizes to mRNA in fetal cells if present in a maternal blood sample. The second prove can comprise (i) a nucleotide sequence corresponding to SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42; (ii) a nucleotide sequence having at least 90% sequence identity to at least 20 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42; or (iii) a nucleotide sequence having at least 80% sequence identity to at least 40 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42. The second probe can correspond to the same or a different fetal cell specific or selective mRNA as the first probe. The kits of the invention can further include diagnostic reagents for determining the gender of the fetal cells or for identifying abnormalities associated with the fetal cells.

In the foregoing fetal cell detection and diagnosis methods and related kits of the present invention, the first probe can be designed to either specifically or selectively hybridize to fetal cells. The first probe is preferably labeled, for example by a radioactive or fluorescent label, a calorimetric reagent, or an enzyme.

The fetal cell detection and diagnosis methods and related kits of the present invention utilize probes having sequences that hybridize to RNAs in fetal cells to a greater extent than RNAs found in non-fetal, e.g., maternal cells in a mixed cell population. Such probes comprise a nucleotide sequence having 20-30 nucleotides with at least 80% sequence identity to a corresponding portion of a fetal cell specific or selective transcript, e.g., SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42. In other embodiments, the nucleotide sequence has at 30-40, 40-60, 60-80, 80-100, 100-150, 150-200, or greater than 200 nucleotides with at least 80% sequence identity to corresponding portion of a fetal cell specific or selective transcript. In various embodiments, the nucleotide sequence has at least 65%, more preferably at least 85%, yet more preferably at least 95% sequence identity to a corresponding portion, e.g., a 30-40, 40-60, 60-80, 80-100, 100-150, 150-200 or greater than 200 nucleotide portion, of a fetal cell specific or selective transcript. In one specific embodiment, the nucleotide sequence has 100% identity to a corresponding portion of a fetal cell specific or selective transcript.

In certain embodiments, a first probe of the invention is less than 40, 50, 100, 200, 300, 400, 500, 1000 or 1500 nucleotides in length. The probe can be an RNA probe, a DNA probe, or a chimeric probe. The probe is preferably single stranded, but can also be partially double stranded.

The fetal cell sought to be detected or diagnosed by the methods and compositions of the present invention is preferably an erythroblast or a trophoblast.

The present invention further provides isolated nucleic acid molecules selected from the group consisting of: (a) a nucleic acid molecule having a nucleotide sequence which is at least 90% identical to the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof; (b) a nucleic acid molecule comprising at least 15 nucleotide residues and having a nucleotide sequence identical to at least 15 consecutive nucleotide residues of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof, (c) a nucleic acid molecule which encodes a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78; (d) a nucleic acid molecule which encodes a fragment at least 10 consecutive amino acid residues of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78; (e) a nucleic acid molecule which encodes a fragment of a polypeptide comprising the amino acid sequence of any SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78; wherein the fragment comprises consecutive amino acid residues corresponding to at least half of the full length of any of said SEQ ID NOs; (f) a nucleic acid molecule which encodes a naturally occurring allelic variant of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78, wherein the nucleic acid molecule hybridizes with a nucleic acid molecule consisting of the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42 under stringent conditions, or a complement thereof. In certain preferred embodiments, the nucleic acid molecule is selected from the group consisting of: (a) a nucleic acid having the nucleotide sequence of any of SEQ ID NOs: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof; and (b) a nucleic acid molecule which encodes a polypeptide having the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78, or a complement thereof. The isolated nucleic acids of the invention can further optionally comprise vector nucleic acid sequences and/or nucleic acid nucleic acid sequences encoding a heterologous polypeptide. The present invention also encompasses prokaryotic and eukaryotic host cells, including but not limited to mammalian and non-mammalian, e.g., bacterial, host cells, which contain the nucleic acid molecules of the invention.

The present invention further provides isolated polypeptides selected from the group consisting of: (a) a fragment of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78; (b) a naturally occurring allelic variant of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78, wherein the polypeptide is encoded by a nucleic acid molecule which hybridizes with a nucleic acid molecule consisting of the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42 under stringent conditions, or a complement thereof; (c) a polypeptide which is encoded by a nucleic acid molecule comprising a nucleotide sequence which is at least 90% identical to a nucleic acid consisting of the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof. In certain embodiment of the invention, the isolated polypeptides have the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78. The polypeptides of the invention can further comprises heterologous amino acid residues. The present invention further encompasses methods for producing a polypeptide selected from the group consisting of: (a) a polypeptide comprising the amino acid sequence of any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78; (b) a polypeptide comprising a fragment of at least 10 contiguous amino acids of the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78; and (c) a naturally occurring allelic variant of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78, wherein the polypeptide is encoded by a nucleic acid molecule which hybridizes with a nucleic acid molecule consisting of the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof under stringent conditions; the methods comprising culturing a host comprising a nucleic acid of the invention under conditions in which the nucleic acid molecule is expressed.

### 3.1. Definitions

SPECIFIC MARKER: a marker (protein, nucleic acid, carbohydrate or other compound) which is found only in or on the target cell type among other cell types in a biological sample of interest. For example, a fetal erythroid cell phenotype specific marker is a marker that is found only on fetal cells of the erythroid lineage, but cannot be detected in/on other cells from the fetus or mother.

SELECTIVE MARKER: a marker that is found predominantly on or in the target cell type, but may be found in other cells as well. For example, fetal hemoglobin is found in fetal blood cells, as well as in a small percentage of maternal blood cells. The selective marker is preferably at least five times more abundant in a target cell relative to a non-target cell in the biological sample of interest, more preferably at least 10, 15, 20, 25, 30, 35, 40, 45 or 50 times more abundant in the target cell relative to a non-target cell in the biological sample of interest, e.g., a maternal blood sample.

SPECIFIC: a nucleic acid used in a reaction, such as a probe used in a hybridization reaction, a primer used in a PCR, or a nucleic acid present in a pharmaceutical preparation, is referred to as “specific” if it hybridizes or reacts only with the intended target. Similarly, a polypeptide is referred to as “specific” if it binds only to its intended target, such as a ligand, hapten, substrate, antibody, or other polypeptide. An antibody is referred to as “specific” if it binds only to the intended target.

SELECTIVE: a nucleic acid used in a reaction, such as a probe used in a hybridization reaction, a primer used in a PCR, or a nucleic acid present in a pharmaceutical preparation, is referred to as “selective” if it hybridizes or reacts with the intended target more frequently, more rapidly, or with greater duration than it does with alternative substances. Similarly, a polypeptide is referred to as “selective” if it binds an intended target, such as a ligand, hapten, substrate, antibody, or other polypeptide more frequently, more rapidly, or with greater duration than it does to alternative substances. An antibody is referred to as “selective” if it binds via at least one antigen recognition site to the intended target more frequently, more rapidly, or with greater duration than it does to alternative substances.

ASSOCIATED: specific or selective.

CORRESPOND OR CORRESPONDING: Between nucleic acids, “corresponding” means homologous to or complementary to a particular sequence or portion of the sequence of a nucleic acid. As between nucleic acids and polypeptides, “corresponding” refers to amino acids of a peptide in an order derived from the sequence or portion of the sequence of a nucleic acid or its complement.

ERYTHROID: an immature cell of the erythroid lineage (i.e., a cell of the erythroid lineage which is not a mature erythrocyte). Erythroid cells include reticulocytes, orthochromatic erythroblasts, polychromatophilic erythroblasts, basophilic erythroblasts, proerythroblasts, colony forming unit-erythroid (CFU-E) and burst forming unit-erythroid (BFU-E).

NUCLEIC ACID OF THE INVENTION: A nucleic acid comprising a nucleotide sequence corresponding to all or a portion of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 and 42, or a variant or derivative thereof.

POLYPEPTIDE OF THE INVENTION: A polypeptide comprising an amino acid sequence corresponding to all or a potion of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, and 78, or a variant or derivative thereof.

FETAL CELL PROBE: A nucleic acid that specifically or selectively hybridizes with a fetal cell RNA or its complement relative to RNAs in other cells in a sample of interest, e.g., non-fetal cells in a maternal blood. A fetal cell probe can be labeled and used for detection of fetal cell RNA. A fetal cell probe can also be in the form of an oligonucleotide useful for PCR amplification of a cDNA corresponding to said fetal cell RNA.

TARGET CELL: a cell of fetal origin in a mixed cell population.

REFERENCE CELLS: a cell that is not a target cell in a mixed cell population.

### 4. BRIEF DESCRIPTION OF THE DRAWINGS

FIG. 1 is a genetic map showing the strategy for converting specific scFv antibody into Fab antibody. Phagemids from a naïve scFv library are cloned and selected for the correct antigen binding characteristics. The immunoglobulin V_{H} and V_{L} regions encoded in the scFv insert are then excised and substituted for the separately translated V_{H} and V_{L} encoding regions in the anti-DOX Fab vector.

FIG. 2 is a three-panel figure showing half-tone reproductions of the analysis of erythroblast antigen. The antigen was immunopurified from a cell extract using Clone 1 anti-erythroblast antibody, subsequently captured on a Nickel absorbant. Upper Panel: Silver-stained polyacrylamide gel; Lower Panels: Western blot at two different exposures. The arrow indicates the position of a specifically identified antigen with an apparent molecular weight of ˜90 kDa.

FIG. 3 is a two-panel figure showing half-tone reproductions of the analysis of erythroblast antigen. The gels were stained with Coomassie Brilliant Blue. Upper Panel: antigen purified by capturing Clone 1 antibody using Nickel absorbant; Lower Panel: antigen purified by capturing biotinylated Clone 1 antibody using streptavidin Dynabeads™. The arrows indicate two bands with apparent molecular weights of ˜90 kDa and ˜78 kDa.

FIG. 4 is a three-panel figure showing half-tone reproductions of the analysis of erythroblast antigen. Upper Panel: Silver-stained polyacrylamide gel; Lower Panels: Western blot from two separate experiments. The analysis compares antigen immunopurified using different erythroblast-specific antibody clones. Clones 18 and 28 appear to recognize ˜90 kDa and ˜78 kDa bands comigrating with those recognized by Clone 1. Clones 17, 22, and 23 appear to identify different bands.

FIG. 5 is a four-panel half-tone figure showing test results for Clone 95 nucleic acid as a probe for fetal cells. The top two panels are a “Southern” (virtual Northern) blot of cDNA from various tissue sources, probed with Clone 95, and shown at two different exposures. The middle panel is an extended Southern analysis using cDNA from a larger panel of tissue samples. The lower panel is a Northern blot of mRNA from different tissues probed with Clone 95.

FIG. 6 is a two-panel half-tone figure showing test results for Clone 369 nucleic acid as a probe for fetal cells. The top panel is a Southern blot; the lower panel is a Northern blot.

FIG. 7 is a six-panel half-tone figure showing the testing of a probe for in situ hybridization. The cells were obtained from human fetal liver blood collected at 16 weeks gestation. The cells were overlaid with the probe at concentrations of 0, 10, and 40 μg/mL (left to right). The staining pattern is consistent with hybridization of the probe with a complementary mRNA sequence present in the cytoplasm.

FIG. 8 is a three-panel half-tone figure showing the results of a genetic analysis according to the invention. Upper left panel shows the phase contrast photomicrograph of cells enriched from a maternal blood sample. The cells were obtained by affinity enrichment using the anti-erythrocyte antibody from Clone 1 in a magnetic activated cell sorting technique. The panel to the right shows the cytoplasmic staining pattern obtained by in situ hybridization using the nucleic acid probe from Clone 369. This specifies which cells in the field are fetal in origin. The lower panel shows the nuclear staining pattern obtained by in situ hybridization using a nucleic acid probe specific for a repeat sequence of Chromosome Y, developed using a different fluorescent marker. The single dot appears in the same cell as the probe, and characterizes the genotype of the fetal cell as having a single Y chromosome.

FIGS. 9A and 9B shows Northern blot data of various tissues obtained from the experiments described in Section 7.2. The probes used in to probe the blots shown in this figure correspond to SEQ ID NO:34 and related clones.

FIGS. 10A and 10B shows Northern blot data of various tissues obtained from the experiments described in Section 7.2. The probes used in to probe the blots shown in this figure correspond to SEQ ID NOs:24 and 27 and related clones.

FIGS. 11A and 11B shows Northern blot data of various tissues obtained from the experiments described in Section 7.2. The probes used in to probe the blots shown in this figure correspond to SEQ ID NO:21 and related clones.

FIGS. 12A and 12B shows Northern blot data of various tissues obtained from the experiments described in Section 7.2. The probes used in to probe the blots shown in this figure correspond to SEQ ID NO:36 and related clones.

FIGS. 13A and 13B shows Northern blot data of various tissues obtained from the experiments described in Section 7.2. The probes used in to probe the blots shown in this figure correspond to SEQ ID NOs:15 and 31 and related clones.

FIGS. 14A and 14B shows Northern blot data of various tissues obtained from the experiments described in Section 7.2. The probes used in to probe the blots shown in this figure correspond to SEQ ID NO:10 and related clones.

FIGS. 15A and 15B shows Northern blot data of various tissues obtained from the experiments described in Section 7.2. The probes used in to probe the blots shown in this figure correspond to SEQ ID NO:41 and related clones.

FIGS. 16A and 16B shows two schematics of the probe signal amplification method that is preferred for detecting fetal cell associated RNAs, as described in Section 8, infra.

FIG. 17 Cord blood cells stained for DAPI (nuclear stain) and L15-1A (cytoplasmic localization) as described in Section 8 below.

FIG. 18 Cord blood cells stained for DAPI (nuclear stain) and L15-1A (cytoplasmic localization) as described in Section 8 below.

FIGS. 19A and 19B Cord blood cells (CB) or bone marrow cells (BM) stained for DAPI (nuclear stain), with or without one or both of J42-4d (cytoplasmic localization), fetal globin epsilon (cytoplasmic localization).

FIG. 20 Cord blood cells stained for DAPI (nuclear, diffuse), gamma and epsilon globins (cytoplasmic) and X and Y chromosomes (subnuclear) using the simultaneous detection methodology of Section 5.9.5.

### 5. DETAILED DESCRIPTION OF THE INVENTION

The present invention provides nucleic acids probes that are useful for identifying a blood cell of fetal origin in a mixed cell population, e.g., a maternal blood sample. The nucleic acid probes are adapted to hybridize with RNA (typically mRNA) present in the fetal cell, or, in some instances, to cDNA reverse transcribed from the RNA. Thus, the fetal cell can be distinguished from maternal cells or other cells that may be present in the mixed population (the “reference cells”), and separated or analyzed in situ. These are referred to in this disclosure as “probes.”

### 5.1. Identification of Fetal Cell Associated RNAs

The present invention provides a methods for discovering nucleic acids the are preferentially or uniquely expressed in fetal cells relative to other cells, e.g., cells of maternal origin, in a mixed cell population. Such nucleic acids are useful for designing probes for identifying fetal cells in a mixed cell population. Polypeptide translation products of such RNAs can be used to prepare antibodies that can be used select for fetal cells in a mixed cell population.

The methods described herein take advantage of the key role the liver plays in the production of fetal cells during gestation. At about 8 weeks, the fetal liver takes over from the yolk sac as the main source of fetal blood cells of all types, including erythroid cells and their precursors. Peak production occurs from about 10-20 weeks of gestation, after which the bone marrow begins to take over. Production of erythroid cells by the liver drops to about 20% of peak levels by week 30, and is virtually absent at term. Fetal liver is therefore an excellent source for RNA species that are more highly expressed in fetal blood cells compared with maternal blood cells. Erythroid cells are easily obtained from fetal liver samples collected between 9-20 weeks of gestation. A preferred collection period is at 16-20 weeks, which corresponds to the highest concentration of nucleated erythroid cells, as a percentage of total cells present. A preferred source is human fetal liver, although other species can be used as a substitute, adjusting gestation times as appropriate. Once a fetal cell associated RNA is identified in a non-human species, the corresponding human homolog can be identified and its expression analyzed to confirm that its expression is associated with cells of fetal rather than maternal origin.

The methods provided herein entail the use of differential expression analysis to identify RNAs that are associated with fetal cells. Generally, the differential expression methods provided herein entail manipulating RNAs obtained fetal cell to either (a) eliminate or reduce RNAs found in cells which are likely to contaminate the test sample or (b) amplify those RNAs which are not found (or found at reduced levels) in cells likely to contaminate the test sample. The differential expression analysis methods identify on a molecular level RNA or cDNA molecules (“tags”) absent from or present at relatively lower amounts in “driver RNA” or “driver cDNA” prepared from “reference cells” (cells which should not be identified by the probe sequence, e.g., maternal blood cells), and present (at relatively higher amounts) in “tester RNA” or “tester cDNA” prepared from target cells (cells which should be identified by the probe sequence, e.g., fetal cells). Such differential expression analysis techniques are discussed in Section 5.1.1, infra.

With respect to the fetal liver as a source of tester nucleic acid, it is preferable that the tissue be chilled very shortly after harvesting, and that mRNA be prepared from the tissue as soon as possible. The erythroid cells are easily separated from hepatic parenchymal cells by gentle manipulation followed by low-speed or gradient centrifugation.

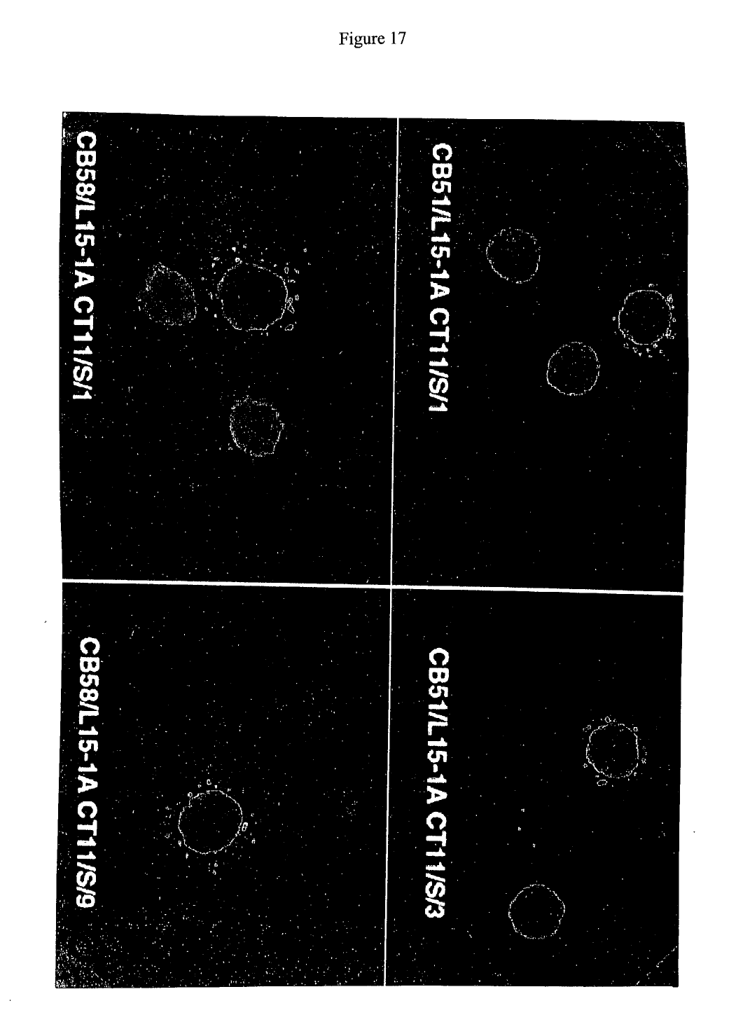

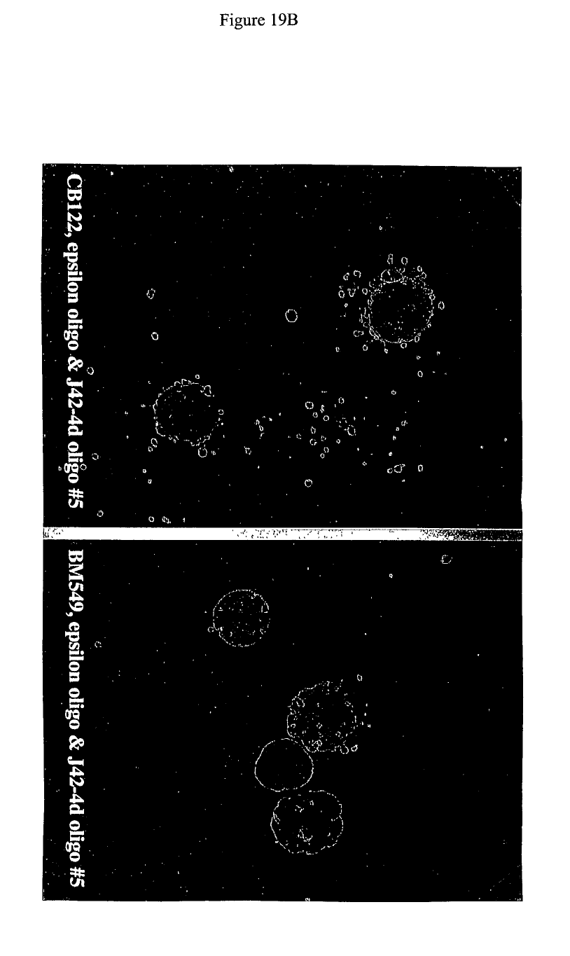

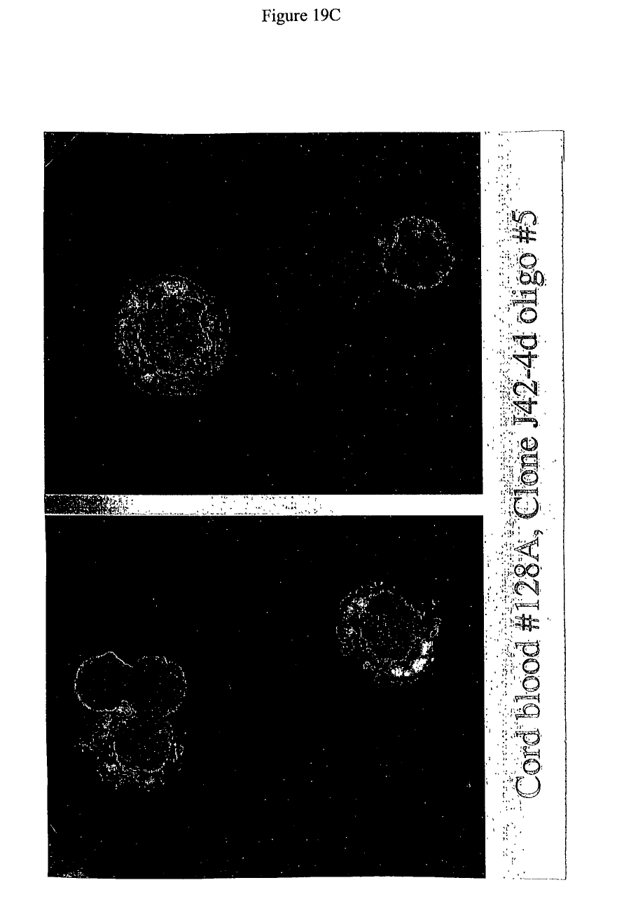

The success of the present methods in identifying probes that are specific to fetal cells or immature erythroid cells is demonstrated in FIGS. 17-19, which show examples of nucleated cells in cord blood cells or human bone marrow cells detected through the presence of DAPI stained nuclei using the DAPI channel (blue), and peroxidase-antibody cascade complexes were detected using TSA-green through the FITC channel (green).

Following differential expression analysis, it is preferably to “validate” the tags as fetal cell specific or fetal cell selective. “Validation” of the specificity or selectivity generally involves clonally expanding each candidate tag, and then evaluating its characteristics by further analysis. Exemplary validation steps to ensure the specificity or selectivity of the fetal cell tags are discussed in Section 5.1.2, below.

Having identified tag sequences with desirable specificity characteristics, further characterization of the RNA or corresponding which is the source of the probe sequence can be performed. Expression patterns can be determined by in situ hybridization using various tissue sections. The full length sequence of the cloned DNA insert can be obtained, and modified probes and primers can be designed. The sequence can be used to pull out overlapping inserts from a cDNA library obtained by SSH or by reverse transcription of fetal mRNA, for example, by the CapFinder™ technique, and the sequence of the entire transcript can be determined. Probe sequences can then be obtained that hybridize anywhere along the transcript. The encoding region can be identified, and the amino acid sequence of the translation product can be predicted. The encoded polypeptides can be recombinantly expressed and used for making antibodies, which antibodies can be used in the fetal cell detection methods of the present invention.

### 5.1.1. Differential Expression Methods

A variety of methods are known in the art for identifying differentially expressed RNAs that can be used to identify fetal cell associated tags, which can then be used to screen for the corresponding full length cDNAs. Additonally, proteome methods may be used to identify polypeptides and their corresponding RNAs or cDNAs that are differentially expressed among maternal and fetal cells. “Differential expression,” as the term is used herein, is understood to refer to both quantitative as well as qualitative differences in expression patterns, e.g., of a gene or genes, between target cells (e.g., fetal cells) and reference cells (e.g., maternal cells).

Methods of differential expression are well-known to one skilled in the art, and include but are not limited to differential display, serial analysis of gene expression (SAGE), nucleic acid array technology, subtractive hybridization, proteome analysis and mass-spectrometry of two-dimensional protein gels. The methods of gene expression profiling are exemplified by the following references describing differential display (Liang and Pardee, 1992, Science 257:967-971), proteome analysis (Humphery-Smith et al., 1997, Electrophoresis 18:1217-1242; Dainese et al., 1997, Electrophoresis 18:432-442), SAGE (Velculescu et al., 1995, Science 270:484-487), subtractive hybridization (Wang and Brown, 1991, Proc. Natl. Acad. Sci. U.S.A. 88:11505-11509), and hybridization-based methods of using nucleic acid arrays (Heller et al., 1997, Proc. Natl. Acad. Sci. U.S.A. 94:2150-2155; Lashkari et al., 1997, Proc. Natl. Acad. Sci. U.S.A. 94:13057-13062; Wodicka et al., 1997, Nature Biotechnol. 15:1259-1267). All such methods are encompassed by the present invention.

In one embodiment, subtractive hybridization is used to identify fetal cell tags. The principle of subtractive hybridization is that cDNAs common to both the target (e.g., fetal) cells and reference (e.g., maternal) cells are selected out by hybridizing to each other, leaving differentially expressed cDNA clones. See Wang et al., 1991, Proc. Nat'l Acad. Sci USA 11505-11509. The subtractive hybridization method of Wang et al. removes commonly expressed cDNA from the experimental and control cDNA pools and thereby enriches for differentially expressed genes.

In another embodiment, one of a number of variations of differential display is used to identify fetal cell tags. See Liang et al., 1992, Science 257:967; Liang et al., 1995; Methods Enzymol. 254:304; U.S. Pat. No. 5,262,311; U.S. Pat. No. 5,599,672. Generally, Liang et al. describe a protocol which involves the reverse transcription of a messenger ribonucleic acid (“mRNA”) population, in independent reactions, with each of twelve anchor primers (T₁₂ MN), where M can be G (guanine), A (adenine) or C (cystosine) and N can be G, A, C or T (thymidine). The resulting single-stranded cDNAs are then amplified by the polymerase chain reaction (hereinafter, “PCR”) using the same anchor primer used for reverse transcription together with an upstream or 5′ decamer of arbitrary sequence. The PCR products, which are labeled by incorporation of tracer amounts of a radioactive nucleotide, are resolved for analysis by denaturating polyacrylamide gel electrophoresis (PAGE). This technique permits the simultaneous visualization of transcripts associated with the reference and target cells, e.g., maternal and fetal cells. Liang et al. postulated that each two-primer combination could amplify only a limited subpopulation of cDNAs, and that the twelve anchor primers together with twenty arbitrary decamers (i.e., 240 PCR reactions) should result in the display of the 3′ termini of all distinct mRNAs that are theoretically expressed in any given cell type (Liang and Pardee, 1992, Science 257:967-971). However, some of the genes identified, although useful for PCR-based identification of fetal cells, are below the limit of detection for in situ hybridization, which is a preferred method for identifying fetal cells according to this invention.

In yet another embodiment, fetal cell tags can be identified by combining subtractive hybridization and differential display. The combined methods involves subtractive hybridization followed by a differential display applied to the subtracted libraries.

In yet another embodiment, fetal cell tags can be identified by representational difference analysis of cDNA, which enriches for differences through rounds of subtraction and selective amplification.

In a preferred embodiment, suppression subtractive hybridization (SSH) is used to amplify candidate probe sequences. Suppression subtractive hybridization, which utilizes a combination of subtractive hybridization and polymerase chain reaction technology, is well known in the art and may even be performed using commercially available kits (Diatchenko et al., 1996, Proc. Natl. Acad. Sci USA 93(12):6025-6030; PCR-select cDNA Subtraction Kit (Clontech), which is based on methods described in U.S. Pat. No. 5,565,340). Generally, mRNA is isolated from the tissue or cell type which produces the tag sequences (e.g., cell/tissue specific or selected mRNA's), then converted into cDNA using any convenient method for production of double-stranded cDNA. cDNA (or a portion of the cDNA) from the tissue or cell type which produces the probe sequences (“tester cDNA”) is digested with a restriction endonuclease to produce appropriate ‘sticky ends’ (single stranded overhangs to which other nucleic acids, such as adaptors, may be annealed), split into two portions (a “first portion” and a “second portion”), and the two portions are modified by the addition of different adaptors of known sequence (e.g., the first portion is modified by addition of a first adaptor and the second portion is modified by the addition of a second, different adaptor). “Driver cDNA” prepared from “reference cells” (e.g., cells which should not be identified by the probe sequence, such as maternal cells in a maternal blood sample) is separately mixed with the modified first and second portions of tester cDNA, and each mixture is denatured and allowed to anneal. Each resulting mixture contains single-stranded tester cDNA, homoduplex tester cDNA, heteroduplex tester/driver cDNA, single stranded driver cDNA, and homoduplex driver cDNA. These mixtures are combined, along with an additional portion of denatured driver cDNA, and allowed to anneal, creating a complex mixture comprising single stranded tester cDNA and portion 1 and portion 2 tester cDNA, homoduplex driver cDNA and portion 1 and portion 2 tester cDNA, heteroduplex portion 1 tester/driver cDNA, heteroduplex portion 2 tester/driver cDNA and heteroduplex portion 1/portion 2 tester cDNA. The ends of the duplex cDNA's are “filled in” using a template-driven reaction (e.g., using DNA polymerase), then amplified using a template-driven amplification process such as the polymerase chain reaction and two primers, a first primer which will anneal to the first adaptor, and a second primer which will anneal to the second adaptor. Only heteroduplex portion 1/portion 2 tester cDNA will be geometrically amplified by the amplification reaction. The end result of SSH is a population of amplified sequences which are derived from RNAs more prevalent in the tester sample than the driver sample.

The SSH process may be reiterated using a different driver cDNA. Reiteration of the SSH process simply requires that the amplified product from the previous round of SSH be digested with a restriction enzyme to produce appropriate sticky ends to the amplified double stranded DNA, preferably using the same restriction endonuclease as in the previous round(s) of SSH. The digested DNA is then split into two portions, modified by the separate addition of different adaptors, and processed. SSH may be reiterated as many times as desired, with any number of driver cDNA samples.

Driver cDNA may be prepared from a variety of sources, including, but not limited to, samples of adult myeloid cells likely to contain a proportion of nucleated erythroid cells, such as adult bone marrow, nucleated cells from adult peripheral blood, and the like. Tumor cells may also be used to prepare driver cDNA. Alternatively, driver cDNA can be prepared from fetal tissues more mature than the source of the tester cDNA, such as fetal liver from later stages gestation.

Preferably, dispersed cells are used for preparation of tester and driver cDNA, although tissues may be used as well. More preferably, cells which have been subfractionated using physicochemical separation techniques or immunoadsorption techniques are utilized for cDNA preparation, particularly for tester cDNA preparation. The cells may also optionally be cultured, although this is generally not recommended, since the expression pattern of the cells may change as a result.

The product of the SSH reaction can be cloned into a suitable vector, thereby constituting a subtracted library from which individual candidate cDNA can be regenerated and validated. The use of the SSH technique permits the preparation of libraries corresponding to a number of fetal cell/reference cell (tester/driver) combinations to determine which combinations of cell types and collection times yield the richest proportion of valid clones.

### 5.1.2. Validation

Described herein are non-limiting examples of validation steps that can be performed on fetal cell associated tags identified by the differential expression analysis methods of the invention. While each of these steps is optional, it is recommended that candidate sequences be evaluated by as many of these criteria as possible. The steps can be performed in any order desired. They are generally listed in order of increasing difficulty or rarity of reagents, and it is generally convenient to perform the steps roughly in the order indicated.

1. PRELIMINARY SEQUENCING. The insert from each randomly selected cDNA clone is PCR amplified, and single-run sequencing of 50-200 nucleotides is performed. The sequence is then compaired against those available in public databases such as GenBank. It is recommended that this be done early in the validation process, to eliminate housekeeping genes, mRNA known to be generously expressed in adult blood cells, and redundant clones. If the sequence contains a single, clear open reading frame, then the orientation of the clone can also be predicted.

2. INITIAL EXPRESSION SCREENING. The cloned DNA is tested in blot analysis of expression patterns in an initial screening panel of fetal and adult cells. It is recommended that this be done using a Southern hybridization technique, using whole cDNA prepared either from mRNA or total RNA of the cells (a “virtual Northern”). The use of cDNA provides a renewable source of material for screening a number of clones. For example, the initial screening panel could include as positives: several fetal blood and fetal erythroid cell samples from fetal liver; and as negatives, adult and fetal liver parenchymal cells and several adult bone marrow cells.

3. EXPANDED EXPRESSION SCREENING. The cloned DNA is then tested for expression patterns using an expanded cell panel: for example, at least five fetal blood and erythroid cells taken at different stages of gestation, and at least three bone marrow and three peripheral blood samples from different adults. Preferred clones show at least 5 times and preferably at least 25 times the expression in the positive samples compared with the negative samples.

4. NORTHERN ANALYSIS. Cloned DNA that pass the preceding expression pattern analysis are preferably retested using mRNA from selected cell populations to verify that the DNA-RNA hybrids form with sufficient specificity to distinguish between the cell populations as a whole. Preferred clones show at least 5 times and preferably at least 25 times the expression in the positive samples compared with the negative samples. Information as to the size of the message and possible alternative splicing may also be obtained. Blots can be stripped and reused for testing of subsequent DNA clones. The blots can also be probed using DNA for housekeeping genes such as GADPH and β-actin, or previously characterized sequences such as transferrin and γ-globin. This permits early elimination of DNA clones hybridizing to transcripts with the same size profile.

5. ORIENTATION AND ABUNDANCE ANALYSIS. Where the DNA is intended to specify fetal cells by hybridizing with mRNA in situ, the correct hybridizing strand should be identified. Orientation analysis is performed by Northern analysis using DNA from the cloned insert prepared as an asymmetric single-stranded probe. Abundance is determined by titration experiments using suitable standards, as are known in the art. The transcript should not only be specific for the desired cell type, it should be sufficiently abundant to provide ready detection of the specified cell according to the intended method.

6. IN SITU mRNA HYBRIDIZATION. Testing for probe sequences intended for in situ hybridization typically includes positive and negative screening using defined cell populations. Positive cell populations for fetal erythroid probes include nucleated erythroid cells from fetal liver, and cultured or uncultured cord blood cells. Positive cells for trophoblast probes are included in cell populations obtained from term placenta and chorionic villae. Negative cell populations include adult peripheral blood myeloid cells and bone marrow cells. Cells of interest in both positive and negative populations are either enriched or counterstained using specific antibody for an important phenotypic marker, such as those described earlier. Preferred DNA probe sequences have a relative rate of true positive to false positive identification of individual cells (estimated from the degree of enrichment of the cell population or the counterstaining) of about 10, 30, or 100 in order of increasing preference. The in situ hybridization analysis will also provide data on the intracellular distribution of the hybridizing transcript. A broad and abundant distribution facilitates most types of subsequent testing. At this stage, technical aspects of hybridization can also be refined; such as the agents used for cell attachment, fixation, and permeabilization; and the labeling, detection or signal amplification methods. Probe specificity can be confirmed by pre-treating the cells with RNAse, and by parallel probing with control sequences.

7. INDEPENDENT CONFIRMATION BY RT-PCR. Wherever possible, it is important to confirm that fetal cells express the probe sequence using a method other than a solid-phase hybridization assay (such as Northern blotting or in situ hybridization). In this test, PCR amplification is conducted using erythroid cells immunoaffinity enriched from fetal liver, or immunoaffinity enriched syncytiotrophoblasts, depending on the nature of the probe. RNA from the cells is reverse-transcribed and used as a template in PCR amplification. Two primers, based on segments of the probe that are about 100-200 base pairs apart, are used in the reaction. PCR amplification is then conducted, and the rate of amplification is determined (measured as the amount of PCR product formed of the correct size after a certain number of cycles). Compared with cells enriched from adult bone marrow or peripheral blood, the rate of amplification is typically at least about 10, 30, or 100 times higher.

8. DIAGNOSTIC TEST RUNS. Model diagnostic analysis is conducted using spiked adult blood samples. Fresh peripheral blood is combined with either blood cells obtained from fetal liver, erythroid cells purified from fetal liver, cultured or uncultured cord blood cells (preferably from about 12 weeks gestation), or cytotrophoblast cells. The fetal cells are from a male fetus and added to an adult female blood sample, so that the Y chromosome can be used to follow specificity. The combined blood sample is then processed by all the intended steps leading up to hybridization with the probe, including density gradient separation and immunoaffinity enrichment. The cells are then processed with the probe according to the intended method to obtain validation. Where the probe is intended for in situ hybridization, the cells are processed accordingly, probed with the probe, and then counterstained for X and Y chromosomal markers. The count of X and Y chromosomes in each cell can also be determined by MGG/benzidine staining. Validation is obtained if the probe correctly distinguishes the fetal cells in the field from the adult blood cells. Similar experiments are then conducted using actual maternal blood samples taken from women carrying a single male fetus. In order of increasing preference, the probe sequence identifies at least about 25%, 50%, 75%, 90%, or 95% of fetal cells in the field. In order of increasing preference, the relative rate of true positive to false positive identification of individual cells (based on Y chromosome counterstaining) is 3, 10, 30, or 100.

### 5.2. Nucleic Acids of the Invention

The present invention provides nucleic acids that relate to the nucleic acids of the invention, i.e., the nucleic acids of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42. Fragments, partially identical homologs, and longer nucleic acids including such sequences are included in the invention. Nucleic acids encoding the polypeptide translation products of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42, and their fragments and derivatives, are also provided.

The nucleic acids of the invention encompass adaptations of the fetal cell associated sequences, particularly those adaptations that facilitate their use in the separation and identification methods described in this disclosure. Additions, deletions, and substitutions of residues can be made for any worthwhile purpose, such as enhancing stability of hybrids formed with the target sequence, adapting towards a consensus of sequence variants, and decreasing cross-reactivity with sequences present in maternal cells. Nucleic acid analogs of this invention include backbone chemistry not found in naturally occurring nucleic acids that improves stability or shelf-life. Labels or moieties for subsequently attaching labels can be attached or inserted into the sequence at any point that does not disturb the desired specificity.

The nucleic acids of the invention, especially those of about 50 nucleotides in length or less, can be conveniently prepared from the sequence data provided in this disclosure by chemical synthesis. Several methods of synthesis are known in the art, including the triester method and the phosphite method. In a preferred method, nucleic acids are prepared by solid-phase synthesis using mononucleoside phosphoramidite coupling units. See, for example, Beaucage et al., 1981, Tetra. Lett. 22:1859; Kumar et al., J. Org. Chem. 49:4905, and U.S. Pat. No. 4,415,732.

Longer nucleic acids can also be prepared by chemical synthesis, but are more typically prepared by amplification or replication techniques. For example, nucleic acids can be amplified by PCR from RNA obtained from fetal tissue or cord blood cells, or from a cDNA library prepared from such tissue. Alternatively, nucleic acids can be amplified by PCR from human genomic DNA libraries. Nucleic acids prepared by any of these methods can be further replicated to provide a larger supply by any standard technique, such as by PCR amplification or molecular cloning.

One aspect of the invention pertains to isolated nucleic acid molecules that encode a polypeptide of the invention or a biologically active portion thereof, as well as nucleic acid molecules sufficient for use as hybridization probes to identify nucleic acid molecules encoding a polypeptide of the invention and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded.

An “isolated” nucleic acid molecule is one which is separated from other nucleic acid molecules which are present in the natural source of the nucleic acid molecule. Preferably, an “isolated” nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free, e.g., at least 60%, 70%, 75%, 80%, 85%, 90%, 95%, 98%, or 99%, free of other cellular material, or culture medium when produced by recombinant techniques, or substantially free of chemical precursors or other chemicals when chemically synthesized. As used herein, the term “isolated” when referring to a nucleic acid molecule does not include an isolated chromosome.

In instances wherein the nucleic acid molecule is a cDNA or RNA, e.g., mRNA, molecule, such molecules can include a poly A “tail”, or, alternatively, can lack such a 3′ tail. Although cDNA or RNA nucleotide sequences may be depicted herein with such tail sequences, it is to be understood that cDNA nucleic acid molecules of the invention are also intended to include such sequences lacking the depicted poly A tails. Where a nucleic acid molecule of the invention is used as a probe, it is preferred the that the probe lacks the polyA tails.

A nucleic acid molecule of the present invention, e.g., a nucleic acid molecule having all or a portion of the nucleotide sequence of SEQ ID NOs: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42, or a complement thereof, can be isolated using standard molecular biology techniques and the sequence information provided herein. Using all or a portion of the nucleic acid sequences of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of SEQ ID NO:1, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42, or a portion thereof.

Moreover, a nucleic acid molecule of the invention can comprise only a portion of a nucleic acid sequence or SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42, a fragment which can be used as a probe or primer (e.g., as described in Section 5.3) or a fragment encoding a biologically active portion of a polypeptide of the invention (e.g., as described in Section 5.4).

The invention further encompasses nucleic acid molecules that differ from the nucleotide sequence of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 due to degeneracy of the genetic code and thus encode the same protein as that encoded by the nucleotide sequence of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42.

In addition to the nucleotide sequences of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42, it will be appreciated by those skilled in the art that DNA sequence polymorphism, including silent polymorphisms and those that lead to changes in the amino acid sequence may exist within a population (e.g., the human population). Such genetic polymorphisms may exist among individuals within a population due to natural allelic variation. An allele is one of a group of genes which occur alternatively at a given genetic locus. As used herein, the phrase “allelic variant” refers to a nucleotide sequence which occurs at a given locus. Such natural allelic variations can typically result in 1-5% variance in the nucleotide sequence of a given gene. Alternative alleles can be identified by sequencing the gene or its corresponding mRNA of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the properties of the nucleic acids (e.g., ability to hybridize to a fetal cell associated RNA) are intended to be within the scope of the invention.

In various embodiment of the present invention, an isolated nucleic acid molecule of the invention is at least 500, 600, 700, 800, 900, 1000, 1100, 1200, 1300, 1400, 1500, 1600, 1700, 1800, 1900 or 2000 nucleotides in length and hybridizes under stringent conditions to the nucleic acid molecule comprising the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42, or a complement thereof.

Accordingly, in other embodiments, an isolated nucleic acid molecule of the invention is at least 50, 100, 200, 300, 400, 500, 600, 700, 800 or 900 nucleotides in length and hybridizes under stringent conditions to the nucleic acid molecule comprising the nucleotide sequence of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42, or a complement thereof.

As used herein, the term “hybridizes under stringent conditions” means conditions for hybridization and washing under which nucleotide sequences at least 70% identical to each other typically remain hybridized to each other. Such stringent conditions are known to those skilled in the art and can be found in Current Protocols in Molecular Biology, John Wiley & Sons, N.Y. (1989), 6.3.1-6.3.6, which is incorporated by reference here in its entirety. A preferred, non-limiting example of stringent hybridization conditions are hybridization in 6×sodium chloride/sodium citrate (SSC) at about 450 C, followed by one or more washes in 0.2×SSC, 0.1% SDS at 50-65° C. Preferably, an isolated nucleic acid molecule of the invention that hybridizes under stringent conditions to the sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42, or a complement thereof, corresponds to a naturally-occurring nucleic acid molecule. As used herein, a “naturally-occurring” nucleic acid molecule refers to an RNA or DNA molecule having a nucleotide sequence that occurs in nature.

In addition to naturally-occurring allelic variants of a nucleic acid molecule of the invention sequence that may exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologues of various species may be non-essential for activity and thus would be likely targets for alteration. Alternatively, amino acid residues that are conserved among the homologues of various species may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78 yet retain biological activity. In one embodiment, the isolated nucleic acid molecule includes a nucleotide sequence encoding a protein that includes an amino acid sequence that is at least about 30%, 35%, 40%, 45%, 50%, 55%, 60%, 65%, 75%, 85%, 95%, or 98% identical to the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, and 78.

An isolated nucleic acid molecule encoding a variant protein can be created by introducing one or more nucleotide substitutions, additions or deletions into the nucleotide sequence of any of SEQ ID NOs: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. A most preferred biological activity for the purposes of the present invention is antigenicity or immunogenicity. Following mutagenesis, the encoded protein can be expressed recombinantly and the activity of the protein, i.e., the ability of be bound by an antibody against the non-mutant protein, can be determined.

The present invention encompasses antisense nucleic acid molecules, i.e., molecules which are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides or more in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a Cech et al. U.S. Pat. No. 4,987,071; and Cech et al. U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675.

PNAs can be used in diagnostic applications. For example, PNAs can be used as antisense or antigene agents for sequence-specific modulation of gene expression by, e.g., inducing transcription or translation arrest or inhibiting replication. In a more preferred embodiment, PNAs are used for fetal cell detection and diagnosis, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a stepwise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide may include other appended groups such as peptides (e.g., for targeting host cell receptors in viva), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

### 5.3. Probes of the Invention

Probe sequences of this invention include those that hybridize with an encoding region or a non-encoding region of the transcript, or span both. Non-encoding regions in some instances are preferred, since they are generally less functionally constrained and less likely to cross-hybridize with other targets. In addition, they may be part of a splice variant which is tissue specific.

The probes of the invention reliably distinguish human fetal cells in a majority of random maternal blood samples. In a single maternal blood sample, optionally enriched using an antibody specific to a fetal cell specific or selective antigen), the probe sequences generally identify at least 25%, and in order of increasing preference, identify about 50%, 75%, 90%, or 95% of fetal cells of a particular phenotype (such as erythroid cells or trophoblast cells) in the population. In a panel of maternal blood samples of mixed ethnic heritage, the relative rate of true positive to false positive identification of individual cells (fetal versus maternal cells identified) is generally at least 3, and is more typically 10, 30, or 100 in order of increased preference.

Preferred probe sequences are those that have minimal cross-reactivity with cells that are abnormally present in certain maternal blood samples due to a disease condition. Relevant diseases include cancer (particularly leukemias, lymphomas, and other myeloid or lymphoid malignancies, and certain endothelial cell and other malignancies that result in sluffing of malignant cells into the circulation), and hemoglobin abnormalities.

The target sequence is described as being “prominent” or “preferentially detected” in fetal cells compared with other cells in the mixed population, if the level of detection (according to the method used) is typically at least 5 times higher, more preferably at least about 25 times higher, and even more preferably at least about 100 times higher than other cells in the population, such as maternal cells of a similar phenotype. Low levels of expression of the sequence are acceptable in maternal cells, as long as the quantitative difference is sufficiently large and consistent to provide a reliable test according to the detection method used. In addition, certain preferred probe sequences have one or more of the properties described in the validation tests of Section 5.1.2.

Of special interest are probes that contain a sequence of consecutive nucleotides that is at least partly identical to a sequence in one of fetal cell associated RNAs of the invention. The length of consecutive nucleotides is generally at least 10 nucleotides, and may be 15, 25, 30, 40, 50, 70, 100, or 200 nucleotides in length. The degree of identity between the region of the probe that corresponds to a nucleic acid of the invention is typically at least 50%, and may be about 70%, 80%, 90%, 95% or 100%. The degree of identity between the region of the probe that corresponds to the fetal cell associated RNA and the corresponding region of the fetal cell associated RNA is typically at least 50%, and may be about 70%, 80%, 90%, 95% or 100%.

One of skill in the art will appreciate that nucleic acids with a longer matching sequence are preferred as more likely to distinguish the target sequence. Longer sequences can be incorporated with more labeling moieties per strand, and need not be as closely identical to the target in order to uniquely identify it. However, shorter sequences generally provide more tissue penetration and more rapid hybridization kinetics. Preferred hybridization probes are 10 to 200 nucleotides in length, more preferably 25 to 100 nucleotides in length. To combine the advantages of a long probe sequence with multiple labeling moieties and the efficiency of shorter-length probes, the probe sequence can be subdivided into nucleic acids of about 25 to 100 residues in length, provided as a reagent mixture. Thus, in certain embodiments of the invention, for a given fetal cell detection assay, a biological sample such as a maternal blood sample is contacted with multiple probes, e.g., of 25-100 nucleotides in length. In one embodiment, the multiple probes comprise nucleic acid sequences that correspond to RNAs transcribed from one gene. The multiple probes can be designed to hybridize to one or more alternative splice forms of the same transcript. In another embodiment, the multiple probes comprise nucleic acids sequences that correspond to RNAs transcribed from more than one gene.

Preferred oligonucleotide probes for use as PCR primers are preferably 10 to 100 nucleotides in length and more typically 15 to 50 nucleotides in length. Individual primers may not necessarily hybridize with unique nucleic acid sequences on the target, and yet still be capable of specifically amplifying a unique sequence when used with a second primer, or when used in a nested amplification reaction with still other primers. The probe/primer typically comprises substantially purified oligonucleotide. In one embodiment, the oligonucleotide comprises a region of nucleotide sequence that hybridizes under stringent conditions to at least about 12, preferably about 25, more preferably about 30, 40, 50, 75, 100, 125, 150, 175, 200, 250, 300, 350 or 400 consecutive nucleotides of the sense or anti-sense sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 and 42 or of a naturally occurring mutant of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 and 42. In another embodiment, the oligonucleotide comprises a region of nucleotide sequence that hybridizes under stringent conditions to at least 400, preferably 450, 500, 530, 550, 600, 700, 800, 900, 1000 or 1150 consecutive oligonucleotides of the sense or antisense sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 and 42 or of a naturally occurring mutant of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 and 42.

The fetal cell probes of the invention may additionally comprise features of the antisense nucleic acid molecules and PNAs described in Section 5.2, supra, including but not limited the inclusion of modified nucleotide residues that impart greater stability on the probe-fetal cell RNA hybrids formed when performing fetal cell detection and/or diagnosis.

Probes for detection of a fetal cell preferably comprise a label group incorporated or attached thereto, e.g., a radioisotope, a fluorescent compound, an enzyme, or an enzyme co-factor.

### 5.4. Polypeptides of the Invention

In addition, to the foregoing nucleic acids, the present invention provide polypeptides encoded by the nucleic acids of the invention. The nucleic acid sequences provide a gateway for analyzing polypeptides encoded by the fetal cell associated nucleic acids. Since the target transcript is preferentially expressed in fetal cells, the polypeptide product is expected to have a similar expression pattern, and may also serve as a marker for fetal cells. Of particular interest are polypeptide products predicted to contain a membrane spanning region, since they are more likely to be expressed at the cell surface. Also of interest are polypeptide products with enzymatic activity, especially for production of a cell-surface marker, or for conversion of a chromogenic substrate. Epitope containing amino acid sequences from the encoding region that are preferably 10, 15, 25, 50 or greater residues in length can also be used to elicit and select specific antibody according to the general methods provided elsewhere in this disclosure. In turn, these antibodies can be used for fetal cell detection or immunoenrichment from a mixed cell population by contacting with the cells under conditions that permit the antibody to bind to the expressed antigen. Although not all nucleic acid molecules of the invention encode a full open reading frame, including a start and stop codon, one skill in the art would recognize that the encoded polypeptides can be recombinantly expressed by inserting the nucleic acid into the appropriate vector with start, stop, and/or translation initiation signals; additionally, such sequences can be encoded by a fusion partner if a polypeptide of the invention is to be expressed in the form of a fusion protein. The following table indicates which polypeptide SEQ ID NOs. correspond to which nucleic SEQ ID NOs. of the invention; where no SEQ ID NO. is given for a corresponding polypeptide is indicative that the nucleic acid in question comprises largely or solely noncoding sequences:

| | Corresponding | Corresponding |
|---|---|---|
| Fetal Cell Specific | Nucleic Acid | Polypeptide |
| Transcript Name | SEQ ID NO. | SEQ ID NO: |
| 1503-7E (tag) | 10 | 43 |
| J42-4d (FL) | 11 | 44 |
| J2r(3) (ASF) | 12 | — |
| J2r(12) (ASF) | 13 | 45 |
| J2r(13) (ASF) | 14 | 46 |
| 305-4G (tag) | 15 | 47 |
| K1-1a (FL) | 16 | 48 |
| K2r/1f(50) (ASF) | 17 | 49, 50, 51 |
| K2r/1f(59) (ASF) | 18 | 52 |
| K(1)157-2A (ASF) | 19 | 53 |
| K3r(HIGH)76 (ASF) | 20 | — |
| 597-10C (tag) | 21 | 54 |
| NT7-T3 (FL) | 22 | 55 |
| N9r/Mf (ASF) | 23 | 56 |
| 334-2C (tag) | 24 | 57, 58 |
| O19r-T3 (FL) | 25 | 59, 60, 61 |
| O1-1a (ASF) | 26 | 62, 63 |
| 332-9E (tag) | 27 | — |
| P60-1a (FL) | 28 | 64, 65, 66 |
| P1-la (ASF) | 29 | 67 |
| P3r(9) (ASF) | 30 | 68 |
| 305-9E (tag) | 31 | 69 |
| R5′-T3 (FL) | 32 | 70 |
| R6r/1-6H (ASF) | 33 | 71 |
| 369-8G (tag) | 34 | — |
| U2f-T3 (FL) | 35 | 72 |
| 305-6G (tag) | 36 | 73 |
| L15-1a (FL) | 37 | 74 |
| L21-1a | 38 | 75 |
| 252 | 39 | — |
| 120r | 40 | 76 |
| Clone-1 | 41 | 77 |
| D19-2g | 42 | 78 |

One aspect of the invention pertains to isolated polypeptides, and biologically active portions thereof, including but not limited to polypeptide fragments suitable for use as immunogens to raise antibodies directed against a polypeptide of the invention. In one embodiment, the native polypeptide can be isolated from cells or tissue sources by an appropriate purification scheme using standard polypeptide purification techniques. In another embodiment, polypeptides of the invention are produced by recombinant DNA techniques. Alternative to recombinant expression, a polypeptide of the invention can be synthesized chemically using standard peptide synthesis techniques.

An “isolated” or “purified” polypeptide or biologically active portion thereof is substantially free of cellular material or other contaminating polypeptides from the cell or tissue source from which the polypeptide is derived, or substantially free of chemical precursors or other chemicals when chemically synthesized. The language “substantially free of cellular material” includes preparations of polypeptide in which the polypeptide is separated from cellular components of the cells from which it is isolated or recombinantly produced. Thus, polypeptide that is substantially free of cellular material includes preparations of polypeptide having less than about 30%, 25%, 20%, 15%, 10%, 5%, 2% or 1% (by dry weight) of heterologous polypeptide (also referred to herein as a “contaminating polypeptide”). When the polypeptide or biologically active portion thereof is recombinantly produced, it is also preferably substantially free of culture medium, i.e., culture medium represents less than about 25%, 20%, 15%, 10%, 5%, 2% or 1% of the volume of the polypeptide preparation. When the polypeptide is produced by chemical synthesis, it is preferably substantially free of chemical precursors or other chemicals, i.e., it is separated from chemical precursors or other chemicals which are involved in the synthesis of the polypeptide. Accordingly such preparations of the polypeptide have less than about 30%, 25%, 20%, 15%, 10%, 5%, 2% or 1% (by dry weight) of chemical precursors or compounds other than the polypeptide of interest.

Biologically active portions of a polypeptide of the invention include polypeptides comprising amino acid sequences sufficiently identical to or derived from the amino acid sequence of the polypeptide (e.g., the amino acid sequence shown in any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, and 78), which include fewer amino acids than the full length polypeptide, and exhibit at least one activity of the corresponding full-length polypeptide. Typically, biologically active portions comprise a domain or motif with at least one activity of the corresponding polypeptide. A biologically active portion of a polypeptide of the invention can be a polypeptide which is, for example, 10, 25, 50, 100 or more amino acids in length. Moreover, other biologically active portions, in which other regions of the polypeptide are deleted, can be prepared by recombinant techniques and evaluated for one or more of the functional activities of the native form of a polypeptide of the invention.

Preferred polypeptides have the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, and 78. Other useful polypeptides are substantially identical (e.g., at least about 45%, preferably 55%, 65%, 75%, 85%, 95%, or 99%) to any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, and 78, and retain the functional activity of the polypeptide of the corresponding naturally-occurring polypeptide yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (, % identity=# of identical positions/total # of positions (e.g., overlapping positions)×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST polypeptide searches can be performed with the XBLAST program, score=50, wordlength=3 to obtain amino acid sequences homologous to a polypeptide molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for polypeptide sequences, or from 1 to 6 for DNA sequences. The default if ktup is not specified is 2 for polypeptides and 6 for DNA. For a further description of FASTA parameters, see http://bioweb.pasteur.fr/docs/man/man/fasta.1.html#sect2, the contents of which are incorporated herein by reference.

The percent identity between two sequences can be determined using techniques similar to those described above, with or without allowing gaps. In calculating percent identity, typically exact matches are counted.

The invention also provides chimeric or fusion polypeptides. As used herein, a “chimeric polypeptide” or “fusion polypeptide” comprises all or part (preferably biologically active) of a polypeptide of the invention operably linked to a heterologous polypeptide (i.e., a polypeptide other than the same polypeptide of the invention). Within the fusion polypeptide, the term “operably linked” is intended to indicate that the polypeptide of the invention and the heterologous polypeptide are fused in-frame to each other. The heterologous polypeptide can be fused to the N-terminus or C-terminus of the polypeptide of the invention.

One useful fusion polypeptide is a GST fusion polypeptide in which the polypeptide of the invention is fused to the C-terminus of GST sequences. Such fusion polypeptides can facilitate the purification of a recombinant polypeptide of the invention.

In another embodiment, the fusion polypeptide contains a heterologous signal sequence at its N-terminus. For example, if a polypeptide of the invention comprises a signal sequence, the native signal sequence can be removed and replaced with a signal sequence from another polypeptide. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.), which may be useful for recombinant expression and/or purification of the polypeptide of the invention.

In yet another embodiment, the fusion polypeptide is an immunoglobulin fusion polypeptide in which all or part of a polypeptide of the invention is fused to sequences derived from a member of the immunoglobulin protein family. The immunoglobulin fusion polypeptides of the invention can be used as immunogens to produce antibodies directed against a polypeptide of the invention.

Chimeric and fusion polypeptides of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

The present invention also pertains to variants of the polypeptides of the invention. Variants can be generated by mutagenesis, e.g., discrete point mutation or truncation. An agonist can retain substantially the same, or a subset, of the biological activities of the naturally occurring form of the polypeptide. An antagonist of a polypeptide can inhibit one or more of the activities of the naturally occurring form of the polypeptide by, for example, competitively binding to an antibody the binds to the native polypeptide of interest.

The polypeptides of the invention can be modified to exhibit reduced or increased post-translational modifications, including, but not limited to glycosylations, (e.g., N-linked or O-linked glycosylations), myristylations, palmitylations, acetylations and phosphorylations (e.g., serine/threonine or tyrosine).

### 5.5. Antibodies of the Invention

The present invention further encompasses the use of antibodies that bind to the polypeptides of the invention for fetal cell detection and diagnostics. The antibodies are preferably monoclonal, and may be multispecific, human, humanized or chimeric antibodies, single chain antibodies, Fab fragments, F(ab′) fragments, fragments produced by a Fab expression library, and binding fragments of any of the above. The term “antibody,” as used herein, refers to immunoglobulin molecules and immunologically active portions of immunoglobulin molecules, i.e., molecules that contain an antigen binding site that immunospecifically binds a polypeptide of the invention. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

An isolated polypeptide of the invention, or a fragment thereof, can be used as an immunogen to generate antibodies using standard techniques for polyclonal and monoclonal antibody preparation. The full-length polypeptide can be used or, alternatively, the invention provides antigenic peptide fragments for use as immunogens. The antigenic peptide of a polypeptide of the invention comprises at least 8 (preferably 10, 15, 20, or 30) amino acid residues of the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, and 78, and encompasses an epitope of the polypeptide such that an antibody raised against the peptide forms a specific immune complex with the polypeptide.

Preferred epitopes encompassed by the antigenic peptide are regions that are located on the surface of the polypeptide, e.g., hydrophilic regions. Hydrophobic regions can be identified by hydropathy plots.

An immunogen typically is used to prepare antibodies by immunizing a suitable subject, (e.g., rabbit, goat, mouse or other mammal). An appropriate immunogenic preparation can contain, for example, recombinantly expressed or chemically synthesized polypeptide. The preparation can further include an adjuvant, such as Freund's complete or incomplete adjuvant, or similar immunostimulatory agent.

In certain embodiments of the invention, the antibodies are human antigen-binding antibody fragments of the present invention and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, may comprise the variable region(s) alone or in combination with the entirety or a portion of the following: hinge region, CH1, CH2, CH3 and CL domains. Also included in the invention are antigen-binding fragments also comprising any combination of variable region(s) with a hinge region, CH1, CH2, CH3 and CL domains. Preferably, the antibodies are human, murine (e.g., mouse and rat), donkey, sheep, rabbit, goat, guinea pig, camelid, horse, or chicken. As used herein, “human” antibodies include antibodies having the amino acid sequence of a human immunoglobulin and include antibodies isolated from human immunoglobulin libraries, from human B cells, or from animals transgenic for one or more human immunoglobulin, as described infra and, for example in U.S. Pat. No. 5,939,598 by Kucherlapati et al.

Antibodies that bind to the polypeptides of the invention may be monospecific, bispecific, trispecific or of greater multispecificity. Multispecific antibodies may be specific for different epitopes of a polypeptide of the invention or may be specific for both a polypeptide of the invention as well as for a heterologous polypeptide. See, e.g., PCT publications WO 93/17715; WO 92/08802; WO 91/00360; WO 92/05793; Tutt, et al., 1991, J. Immunol. 147:60-69; U.S. Pat. Nos. 4,474,893; 4,714,681; 4,925,648; 5,573,920; 5,601,819; Kostelny et al., 1992, J. Immunol. 148:1547-1553.

The present invention encompasses the use of derivatives of the antibodies of the invention that are modified, i.e., by the covalent attachment of any type of molecule to the antibody such that covalent attachment does not prevent the antibody from binding to a polypeptide of the invention. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other polypeptide, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies of the invention can be produced by various procedures well known in the art. For example, polypeptides of the invention can be administered to various host animals including, but not limited to, rabbits, mice, rats, etc. to induce the production of sera containing polyclonal antibodies specific for the polypeptide. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed., 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a non-limiting example, mice can be immunized with a polypeptide of the invention or a cell expressing a polypeptide of the invention. Once an immune response is detected, e.g., antibodies specific for the polypeptide of the invention are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding the polypeptide of the invention. Ascites fluid, which generally contains high levels of antibodies, can be generated by injecting mice with positive hybridoma clones.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles which carry the nucleic acid sequences encoding them. In a particular embodiment, such phage can be utilized to display antigen binding domains expressed from a repertoire or combinatorial antibody library (e.g., human or murine). In phage display methods, functional antibody domains are displayed on the surface of phage particles which carry the nucleic acid sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 binding domains expressed from phage with Fab, Fv or disulfide stabilized Fv antibody domains recombinantly fused to either the phage gene III or gene VIII protein. Phage expressing an antigen binding domain that binds to polypeptide of the invention or a binding portion thereof can be selected or identified with antigen e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology, 191-280; PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 1992, 12(6):864-869; and Sawai et al, 1995, AJR134:26-34; and Better et al., 1988, Science 240:1041-1043 (said references incorporated by reference in their entireties).

Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; and Skerra et al., 1988, Science 240:1038-1040. For some uses, including in vivo use of antibodies in humans and in vitro proliferation or cytotoxicity assays, it is preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 1985, 229:1202; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entirety. Humanized antibodies are antibody molecules from non-human species antibody that binds the desired antigen having one or more CDRs from the non-human species and framework and constant regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., 1988, Nature 332:323, which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology, 1991, 28(4/5):489-498; Studnicka et al., 1994, Protein Engineering 7(6):805-814; Roguska. et al., 1994, PNAS 91:969-973), and chain shuffling (U.S. Pat. No. 5,565,332).

Completely human antibodies can be used in the present methods. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which express human immunoglobulin genes. For example, the human heavy and light chain immunoglobulin gene complexes may be introduced randomly or by homologous recombination into mouse embryonic stem cells. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce useful IgG, IgA, IgM and IgE antibodies. For an overview of this technology for producing human antibodies, see, Lonberg and Huszar, 1995, Int. Rev. Immunol. 13:65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., 1994, Bio/technology 12:899-903).

Further, antibodies to the polypeptides of the invention can, in turn, be utilized to generate anti-idiotype antibodies that “mimic” the polypeptides of the invention using techniques well known to those skilled in the art. (See, e.g., Greenspan & Bona, 1989, FASEB J. 7(5):437-444; and Nissinoff, 1991, J. Immunol. 147(8):2429-2438).

### 5.6. Recombinant Vectors and Host Cells

Another aspect of the invention pertains to vectors, preferably expression vectors, containing a nucleic acid encoding a polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant polypeptide.

Examples of suitable inducible non-fusion E. coli expression vectors include pTrc (Amann et al., (1988) Gene 69:301-315) and pET Id (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 1 id vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant polypeptide expression in E. coli is to express the polypeptide in a host bacteria with an impaired capacity to proteolytically cleave the recombinant polypeptide (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of polypeptides in cultured insect cells (e.g., Sf 9 cells) include the pAc series (Smith et al. (1983) Mol. Cell Biol. 3:2156-2165) and the pV_{L} series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the mouse hox promoters (Kessel and Gruss (1990) Science 249:374-379) and the beta-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

In another embodiment, the expression characteristics of an endogenous (e.g., J42-4d or D19-2 g gene) within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene (e.g., J42-4d or D19-2 g gene) and controls, modulates or activates. For example, endogenous J42-4d or D19-2 g genes which are normally “transcriptionally silent”, i.e., a J42-4d or D19-2 g gene which is normally not expressed in maternal erythroid cell, or are expressed only at very low levels in a cell line or microorganism, may be activated by inserting a regulatory element which is capable of promoting the expression of a normally expressed gene product in that cell line or microorganism. Alternatively, transcriptionally silent, endogenous J42-4d or D19-2 g genes may be activated by insertion of a promiscuous regulatory element that works across cell types.

A heterologous regulatory element may be inserted into a stable cell line or cloned microorganism, such that it is operatively linked with and activates expression of endogenous genes, using techniques, such as targeted homologous recombination, which are well known to those of skill in the art, and described e.g., in Chappel, U.S. Pat. No. 5,272,071; PCT publication No. WO 91/06667, published May 16, 1991.

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

### 5.7. Methods for Fetal Cell Enrichment

The invention provides methods of identifying and diagnosing fetal cells in a sample. Most commonly, the fetal cells will be present in a maternal blood sample, where the fetal cells comprise an extremely low percentage of the total cells present. As mentioned above, prior to carrying out the fetal cell detection and diagnosis methods of the present invention, it is preferable that the biological sample, e.g., maternal blood sample, that will be subject to detection or diagnosis is enriched for rare fetal cells.

A mixed blood sample may be enriched for fetal cells by fractionation. Fractionation methods include density gradient centrifugation and differential lysis. For example, density gradients can be used to remove maternal red blood cells and lymphocytes (see, e.g., Durrant et al., 1996, Early Hum Dev 47 Suppl:S79-83). Similarly, maternal blood cells can be removed from a maternal blood sample by differential lysis of the maternal cells, as described by Furbetta et al., 1980, Br J Haematol 44(3):441-50.

The foregoing fractionation techniques can be done as an alternative or, more preferably, in addition to the immunoenrichment methods described in Section 5.7.1 below.

### 5.7.1. Immunoenrichment

In a preferred embodiment, the fetal cell enrichment step utilizes a fetal cell-associated or maternal cell-associated antibody (an “immunoenrichment” step).

Immunoenrichment can be positive immunoenrichment, whereby the mixed cell population of interest is contacted with a fetal cell-associated antibody and cells bound to the antibody are selected for. Preferably, the antibodies are directed to fetal blood cell associated antigens or trophoblast associated antigens. The antibodies are preferably specific or selective for antigens which are not found on maternal blood cells in a maternal blood sample. More preferably, the antibodies are specific or selective for a fetal blood cell-specific or trophoblast-specific antigen. One exemplary antibody for use in immunoenrichment is Clone 1 which is specific for a fetal erythroid cell antigen. An antibody comprising the heavy chain or light chain of Clone 1, or one or more heavy or light chain CDRs of Clone 1, can also be used.

Immunoenrichment can also be negative immunoenrichment, whereby the mixed cell population of interest is contacted with a maternal cell-associated antibody and cells bound to the antibody are selected against. Preferably, the antibodies used in negative immunoenrichment have little to no binding to fetal erythroid and trophoblast cells.

Immunoenrichment may be carried out using any appropriate methodology known in the art. Preferred methods include fluorescence-activated cell sorting (FACS), immunomagnetic technologies, immunoprecipitation methods, and solid-phase separation methods (e.g., panning). Generally, the antibody used for immunoenrichment is modified in a way that allows separation of cells with bound antibody from cells without bound antibody. In the case of FACS, the immunoenrichment antibody is labeled with a fluorescent compound (or secondarily labeled with a fluorescent compound), incubated with the maternal sample, then the cells of the maternal sample are separated into labeled and unlabeled fractions using an automated sorter. Immunoenrichment using immunomagnetic technology generally involves binding cells in the maternal sample with an immunoenrichment antibody primarily or secondarily labeled with paramagnetic or ferromagnetic particles, followed by separation of labeled cells with a magnetic field. Other useful techniques involve binding, adsorbing, or otherwise linking an immunoenrichment antibody to a solid phase, such as a bead or a plastic substrate, binding the antibody to cells in a maternal sample, and retaining bound cells on the basis of the properties of the solid phase (e.g., by collection of beads). Preferred forms of immunoenrichment antibody include immunoenrichment antibodies which are haptenized, directly labeled with a fluorescent dye, adsorbed to magnetic particles, linked to a suspendable solid phase such as beads, or adsorbed to a solid phase such as a plastic dish.

Normally, the immunoenrichment step comprises incubating the sample containing the mixed cell population with an immunoenrichment antibody for a sufficient period of time to allow binding of the immunoenrichment antibody to target cells present in the sample. The incubation period is typically from about 10 or 15 minutes up to several hours. The incubation may be carried out at elevated temperatures (e.g., about 30° to 37° C), at room temperature (RT, approximately 19° to 22° C.) or at reduced temperatures (e.g., from about 4° to about 15° C.). The incubation is normally carried out in a physiologically-acceptable solution containing a pH buffer, salts, and optionally containing dextrose and/or blocking agents such as serum albumin, gelatin, and the like.

The steps following incubation of the maternal sample with the immunoenrichment antibody will depend on the immunoenrichment antibody and any modifications thereto, as will be apparent to one of skill in the art. Generally, the sample will be processed to separate cells bound to the immunoenrichment antibody from cells not bound to the immunoenrichment antibody.

When magnetic particles are used, the sample is subjected to a magnetic field, which is generally oriented to segregate the antibody-bound cells to the wall of the incubation vessel, and the incubation solution, including any bound, is removed. Where positive immunoenrichment is used, i.e., the antibody bound cells are the fetal cells of interest, the incubation solution is discarded. Where negative immunoenrichment is used, the incubation solution will contain the fetal cells of interest and antibody-bound cells are discarded. Similarly, for suspendible solid phase-based systems, the suspendible solid phase is allowed to settle. The supernatant, including unlabeled cells, is removed where a positive immunoenrichment antibody is used, and collected for further processing where a negative immunoenrichment antibody is used. Where the immunoenrichment antibody is adsorbed to a non-suspendible solid phase (e.g., the bottom of a plastic dish), unbound cells and incubation solution are simply removed or collected, as desired. For positive immunoenrichment, the cells thus separated may be washed by simply resuspending the sample (where magnetic or suspendible solid phase technology is used), or simply adding additional buffer (where non-suspendible solid phase technology is used) and repeating the separation procedure. Preferably the separated cells are washed at least once.

When FACS is used for separating antibody-bound and non-bound cells, the cells are normally processed to render bound cells detectable, if the immunoenrichment antibody is not primarily labeled. For positive immunoenrichment, such processing generally involves washing away any unbound immunoenrichment antibody, then incubating with a detection reagent (e.g., rhodamine-derivatized avidin for a biotinylated immunoenrichment antibody, or a labeled secondary antibody of appropriate specificity for an unmodified immunoenrichment antibody), washing again, then FACS processing.

Washing is typically carried out using the solution which was used for the antibody incubation, minus the antibody, although simple buffered solutions such as phosphate buffered saline (PBS) and tris buffered saline (TBS) may also be used. The cells of the maternal sample are typically washed several times, generally about 2-3, although a larger or smaller number of washes may be used as long as sufficient excess immunoenrichment antibody is removed and the cells are not unduly damaged by the washing procedure.

Optionally, additional rounds of immunoenrichment are utilized to further enrich the mixed cell population for fetal cells. The increase in enrichment of fetal cells in the maternal sample with additional rounds of immunoenrichment must be balanced against loss of cells due to processing during immunoenrichment. Preferably, immunoenrichment is performed in 1 to 4 rounds, 1 to 3 rounds, or 1 to 2 rounds. Where more than one round of immunoenrichment is carried out, it is preferred that different immunoenrichment antibodies are used for each round of immunoenrichment.

Depending on the technology used for positive immunoenrichment, it may be desirable to “release” antibodies bound to the fetal cells enriched from the maternal sample after immunoenrichment is completed and/or between rounds of immunoenrichment. Release of bound antibodies is generally accomplished by altering pH or ionic conditions in the fluid medium.

### 5.7.2. Methods for Identifying Fetal Cell Associated Antibodies

The selection step of the instant methods for enriching fetal cells in a sample prior to fetal cell detection and/or diagnosis is preferably performed using one or more antibodies specific for a specific or selective marker on the target cell. The instant invention provides antibodies specific for fetal erythroid specific markers and selective markers, which can be used in the selection step, as well as methods for isolating new target cell specific and selective antibodies.

The inventors have discovered that fetal liver is an excellent source for erythroblast cells expressing suitable markers for antibody development. Human fetal liver samples are preferably collected between about 10-18 weeks of gestation, taking care to chill the tissue very shortly after harvesting. Preferably, the fetal livers experience no more than 15 minutes of warm hypoxia. The fetal livers are dissociated by gentle mechanical dispersion (e.g., trituration or pressing between sterile glass plates), and erythroid cells are separated from hepatic parenchymal cells by, for example, low-speed or gradient centrifugation.

The cells may then be used as the “target preparation” for immunization or antibody selection. It is recommended that the cells be used immediately and without further manipulation, so as not to affect antigen display. However, cultured cells or preserved cells with or without mild fixation may also be used as the target preparation, and it is also possible to use cellular extracts, purified membranes, or antigen fractions as the target preparation.

Antibodies can be raised against antigens in the target preparation by immunizing animals with the target preparation. Preferably, the animals have been previously tolerized to adult human blood cells, preferably nucleated red blood cells isolated from adult human peripheral blood. Serum may be collected from such immunized animals, and polyclonal antibodies may be purified from the serum. For methods of antibody production, see generally the Handbook of Experimental Immunology (D. M. Weir & C. C. Blackwell, eds.); and Current Protocols in Immunology (J. E. Coligan et al., eds., 1991).

When animals are immunized with the target preparation, it is preferable to prepare monoclonal antibodies. Monoclonal antibody production is well known in the art, and generally involves isolation of immunoglobin-producing cells (or immunoglobin producing cell precursors) from immunized animals. The isolated cells are immortalized by, for example, fusion with a myeloma cell line which does not produce immunoglobin or by transformation with Epstein-Barr virus (EBV). Clones of immortalized cells which produce antibodies of interest are isolated by screening the clones (or supernatant from cultures of the clones) against an antigen of interest, typically the target preparation. Methods of monoclonal antibody production can be found in, for example, U.S. Pat. Nos. 4,491,632, 4,472,500, and 4,444,887, and Galfre et al. (1981, Meth. Enzymol., 73B:3-46).

In a particularly preferred method, the target preparation is used to select antibody-producing clones from an established library of immunocompetent cells or particles. Preferably, the library is a “naïve” library, which means that it is not biased by previous immunization events. The preferred naïve library will either be a germ-line library, or a library prepared from a young, immunologically naïve animal neither tolerized nor sensitized against any foreign antigen. Especially preferred is a “germ-line” library, in which an array of variable regions (usually V_{H} and V_{L}) are obtained in germ-line form and assembled in the library in random heterodimeric combinations. The variable regions in a germ line library will not have gone through the somatic mutation events that normally occur in cells of the B lymphocyte lineage during affinity maturation. The number of theoretical combinations of germ-line V_{H} and V_{L} regions (the product of the numbers of encoded V_{H} and V_{L} variants) can exceed 10⁹, 10¹¹, or even 10¹³, especially when encoding sequences from a large plurality of out-bred individuals of the same species are used in preparing the library. Higher numbers of V_{H}-V_{L} combinations are preferred, since this increases the probability of obtaining a specific antibody with a higher affinity. A key advantage of a germ line library is that it will not have immunological blind spots due to tolerization for self antigens, as would be present in a library obtained, say, from the rearranged immunoglobulin genes of a mature B lymphocyte population. Thus, antibodies against rare self-antigens are obtainable. For preparation of germ line antibody libraries, see generally Marks et al. (1996, N. Engl. J. Med. 335(10):730-733), and McGuinness et al. (1996 Nat. Biotechnol. 14(9):1149-1154). Particularly preferred is the Griffiths library, described in Griffiths et al. (1993, EMBO J. 12(2):725-734), in which single-chain variable regions (scFv) are displayed on phage.

To perform the selection, the cells or viral particles are contacted with the target preparation under conditions that permit the antibody to bind the cells if they display an antigen binding site specific for a cell antigen, as will be undersood by one of skill in the art. The bound cells or viral particles are separated from unbound cells or viral particles, then the bound cells or viral particles are released from the target preparation and the process is preferably repeated several times. Negative selection can optionally be conducted by contacting with mature erythrocytes or other non-erythroblasts and collecting the unbound cells or viral particles. The cells or viral particles can be replicated at various points during selection if necessary to replenish the supply.

Selected antibodies are preferably further validated by clonally replicating the particles or cells expressing the selected antibody, then testing the clones (or the antibody produced by them) in positive and negative screens. Positive screening may be accomplished by testing the antibodies with cells or antigen preparations which the antibody should bind to, such as erythroblasts, preferably fetal erythroblasts. Negative screening may be accomplished by testing the antibodies against cells or antigen preparations to which the antibody should not react, such as mature erythrocytes, monocytes, granulocytes, or lymphoid cells from periperal blood. Optionally, the antibodies may be additionally negatively screened against erythroblasts and bone marrow from adults. Antibodies that react in the positive screen and do not react in the negative screen may be used in the fetal cell enrichment methods of the invention, but are preferably further selected and/or characterized.

Preferably, the antibodies which pass validation testing are also tested in an immunoaffinity purification assay, if such an assay has not been part of the validation testing. As will be understood by one of skill in the art, any given antibody may have varying effectiveness across different assays. For example, an antibody which is highly efficacious in immunostaining may perform poorly in quantitative immunoassays such as an ELISA. Accordingly, it is recommended that antibodies which pass validation testing be further tested for the ability to enrich erythroblasts from amniotic cord blood samples. Optionally, antibodies which perform well in enriching erythroblasts from amniotic cord blood samples are further tested for the ability to enrich fetal erythroblasts from a maternal blood sample.

At any time during or following the selection or validation process, further adaptations of the antibody molecule both within and outside the variable region can be conducted. It has been found that a proportion of scFv antibodies, when not expressed on the surface of a phage, undergo denaturation upon incubation for several hours or days at 37° C. This is attributed to a weak affinity between the V_{H} and V_{L} chains along the interface. Antibodies with this property can be tested while attached to the phage, or converted to another construct such as an antibody consisting of or containing an Fab fragment. The V_{H} and V_{L} interface is stabilized in the Fab due to interaction of the CL and CH1 immunoglobulin domains. Conversion of genetic constructs encoding scFv to those that encode Fab is a matter of standard genetic manipulation, and is illustrated herein.

The antibodies of the invention include antibody molecules having the V_{H} or V_{L} sequence of the exemplary antibodies, with or without modifications in the amino acid sequence. Acceptable modifications to the V_{H} or V_{L} sequence of the exemplary antibodies include amino acid insertions, deletions, and substitions, so long as the modified antibodies retain the specificity of the ‘parent’ antibody (e.g., the antibody upon which the modified antibody is based). A wide range of alterations of the variable region framework are typically available that do not compromise specificity. Alterations in buried residues, interface residues, and antigen-binding residues are less frequent, as are non-conservative substitutions and excisions that affect folding, but all such alterations are permissible as long as the specificity of the parent antibody is maintained. Methods used for ‘humanization’ of non-human antibody variable regions, such as those disclosed in International Patent Applications Nos. WO 94/11509 and WO 96/08565 may be applied to the antibodies of the invention, or may simply be used as guides for selecting residues to be altered. Alterations are also permitted that improve specificity, including mutations in the CDR and substitution of either the V_{H} or V_{L} with a variable region chain from another antibody.

Certain embodiments of the invention are antibodies having the complementarity determining regions (CDRs) of either the V_{H} or the V_{L} (preferably both) that are homologous to those of one of the exemplary antibodies described herein. Preferably, the homologous CDRs contain no more than about 5 alterations per V_{H} or V_{L} chain in comparison with the prototype.

Antibodies having any of the alterations indicated above can be identified as having desirable specificity without undue experimentation, by simply conducting binding or purification assays similar to that used to validate the specificity of the parent molecule, as illustrated herein.

Certain embodiments of the invention comprise antibodies that compete with one of the exemplary antibodies for binding to an antigen preferentially expressed on human erythroblasts. Such antibodies can be identified, for example, by adapting any binding or validation assay for the parent molecule to a competition format. In a preferred example, the exemplary antibody is used for immunofluorescent labeling or immunoaffinity purification of erythroblasts in a mixed cell population as already described. However, the cells are preincubated or the separation step is carried out in the presence of the antibody being tested in an unlabeled form. Ability to compete with the exemplary antibody is indicated by decreased effectiveness of the exemplary antibody in labeling or purification. Competition can also be assayed by antibody binding in a blot or antigen immunoassay format.

Certain embodiments of the invention comprise antibodies that bind the same antigen as one of the exemplary antibodies. Such antibodies can be identified, for example, by competition assays using one of the exemplary antibodies and purified antigen. Included are antibodies that are specific for an erythroblast antigen with an apparent molecular weight of 78 kDa or 90 kDa as determined by polyacrylamide gel electrophoresis in sodium dodecyl sulfate (SDS-PAGE) under disulfide reducing conditions.

Particular antibodies of this invention can be prepared based on the amino acid sequence data provided in this disclosure, incorporating any desired amino acid deletions, additions, or substitutions. Peptide synthesis and assembly is one possible approach, but it is usually more convenient to prepare proteins of the length of variable region chains by expressing a nucleic acid encoding it in a suitable prokaryotic or eukaryotic host cell. One example is the phagemid vector VODOX1, which can be used as a backbone for expressing V_{H} and V_{L} polypeptide sequences as an Fab fragment. The construct can encode recognition sites such as polyhistidine or a c-myc tag that permit later purification by affinity methods. Alternatively, antibody can be purified from cell supernatants, lysates, or ascites fluid by a combination of traditional biochemical separation techniques, such as amonium sulfate precipitation, ion exchange chromatography on a weak anion exchange resin such as DEAE, hydroxyapatite chromatography, and gel filtration chromatography.

The antibodies used in the invention have a variety of utilities, including enriching cells in mixed samples, purification of antigens, and imaging, detection, identification, and quantitation of cells.

The erythroid cell antibodies of the invention may be used for direct or indirect immunostaining. Accordingly, the antibodies may be used for imaging, detecting and/or identifying erythroid cells in biological samples, by contacting cells in a sample with an antibody of the invention, permitting formation of a stable complex, and then visualizing cells bearing the stable antigen-antibody complex by any method known in the art. The antibodies may also be used to quantitate erythroid cells in a sample, using the antibodies in a quantitative immunoassay. To the extent that the target antigen is also expressed on cells that are dedifferentiating during oncogenesis, the antibody may also be used to image, detect, identify and/or quantitate such cells.

Antibodies of this invention can be used to raise anti-idiotypes for erythroid antigens, according to any method known in the art. Generally, anti-idiotype antibodies are prepared by using an anti-erythroid cell antibody of the invention as an immunogen or to select antibody producing particles, for example from a phage library. Selection of the anti-idiotype clones is done using the anti-erythroid cell antibody as a positive selector, and using antibodies of unrelated specificity, but generally of the same isotype, as negative selectors. Validation of initially selected clones is performed by inhibition experiments, in which desired clones block binding between anti-erythroid cell antibody and either erythroid cells or the target antigen. Anti-idiotype clones may be further selected for their ability to elicit a specific anti-erythroid cell antibody in a naïve mammal, or selecting a specific anti-erythroid cell antibody from an antibody library. Anti-idiotypes can then be used to obtain additional clones of anti-erythroid cell antibodies.

The antibodies of the instant invention are particularly advantageous for enriching erythroid cells in biological samples. A mixed cell population containing erythroid cells is contacted with an antibody of the invention under conditions that permit the antibody to bind to an erythroid cell antigen and form a stable complex, then the cells bearing the stable antigen-antibody complex are separated from cells not bearing the stable complex. The mixed cell population may be any population containing erythroid cells, including bone marrow cells and other blood cell progenitor and precursor populations. Of particular interest are obtaining fetal erythroid cells from maternal blood for purposes of prenatal genetic diagnosis, as described herein.

Antibodies of this invention can also be used to identify, purify, or characterize their target antigen. The Examples provide an illustration of the immunoaffinity purification of a 78-90 kDa antigen from a lysate of erythroblasts, using the antibody produced by the antibody referred to as Clone 1, whose heavy and light chain coding sequences are SEQ ID NOs:8 and 9, respectively. The expression pattern of this antigen along the erythrogenic pathway is compared with that of other cell markers in Table

TABLE 1
Relative Expression (-to ✓✓)

| | | CD71 | | | | Clone 1 |
|---|---|---|---|---|---|---|
| Cell Phenotype | Hemoglobin | (TfR) | CD45 | CD36 | Glycophorin A | Antigen |
| Proerythroblast | — | — | — | ✓ | ✓ | ✓✓ |
| Basophilic Erythroblast | — | ? | — | ✓ | ✓ | ✓✓ |
| Polychromatophilic | ✓✓ | ✓ | — | ✓ | ✓ | ✓✓ |
| Erythroblast | | | | | | |
| Orthochromatic | ✓✓ | ✓ | — | — | ✓✓ | ✓✓ |
| Erythroblast | | | | | | |
| Reticulocyte | ✓✓ | ✓ | — | — | ✓✓ | ✓ |
| Mature Erythrocyte | ✓✓ | ✓ | — | — | ✓✓ | — |
| Other Blood Cells | — | ✓ | ✓✓ | ✓ | — | — |

Once the amino acid sequence of the target antigen is obtained, the full length antigen or a fragment of the antigen can be prepared synthetically for further use.

Generally, polypeptides can be prepared either by chemical synthesis, or by expression of a nucleic acid encoding it in a cell-free translation system or in a host cell. Short polypeptides of about 30 or fewer amino acids in length are conveniently prepared from sequence data by chemical synthesis. A preferred method is solid phase synthesis, in which the C-terminal amino acid is attached to a solid phase and the peptide is grown towards the N-terminal, as is well known in the art, using iterative cycles of deprotection of the growing protein on the solid phase and coupling the next amino acid, followed by cleavage of the completed peptide from the solid phase and deprotection of the amino acid side chains. Recombinant expression is the preferred method for production of longer polypeptides. A large variety of recombinant expression systems are known in the art, utilizing a variety of constructs and host cells. Generally, a nucleic acid encoding the desired protein is operatively linked to a suitable promoter in an expression vector, and transfected into a suitable host cell. The host cell is then cultured under conditions that allow transcription and translation of the protein, which is subsequently recovered and purified.

The epitope to which a particular antibody binds can be mapped by preparing fragments and testing the ability of the antibody to bind. For example, sequential peptides of 12 amino acids are prepared covering the entire sequence, and overlapping by 8 residues. The peptides can be prepared on a nylon membrane support by F-Moc chemistry, using a SPOTSO kit from Genosys according to manufacturer's directions. Prepared membranes are then overlaid with the antibody, washed, and overlaid with β-galactose conjugated anti-human IgG. The test is developed by adding the substrate X-gal. Positive staining indicates an antigen fragment recognized by the antibody.

Purified erythroblast antigens and antigen fragments may in turn be used to prepare additional erythroblast-specific antibodies according to the general techniques already described.

Antibodies against a fetal cell associated or specific antigen may be derivatized for use in the methods of the present invention. Details of production of antibody fragments and derivatives are well known in the art and may be found, for example in, “Antibody Engineering,” 2nd edition (C. Borrebaeck, ed., Oxford University Press, 1995) and “Immunoassay” (E. P. Diamandis & T. K. Christopoulos, eds., Academic Press, Inc., 1996). The term “antibody” also refers to fusion polypeptides comprising an antibody of the invention and another polypeptide or a portion of a polypeptide (a “fusion partner”), such as an affinity tag, an enzyme or other fusion partner.

For use in certain aspects of the instant invention, antibodies may be “primarily” or “secondarily” labeled. A primarily labeled antibody is an antibody which is directly conjugated to a composition which permits detection of the antibody. A secondarily labeled antibody is an antibody which is bound to a detection composition through at least one intermediate composition. For example, an antibody may be primarily labeled by covalent linkage to an enzyme or fluorescent molecule or by adsorption to a magnetic particle. A secondarily labeled antibody may be unmodified and labeled by binding a labeled antibody-binding protein (such as Protein A, Protein G, or an anti-immunoglobin antibody which may be primarily or secondarily labeled itself), or modified, and labeled by a compound which specifically binds the modification (e.g., covalent modification with a hapten such as biotin followed by labeling with labeled hapten binding protein such as avidin or streptavidin).

### 5.8. Methods of Fetal Cell Detection

The present invention provides methods of detecting rare fetal cells in a mixed cell population. Such methods utilize the nucleic acids identified herein as being selectively or specifically expressed in fetal cells relative to other cell types in the mixed cell populations of interest.

Identification of fetal cells with nucleic acid probes is normally carried out using an in situ approach, generally fluorescent in situ hybridization (FISH), although in situ amplification methods are also contemplated. For FISH, a nucleic acid probe is modified (or synthesized with modified nucleotides) so that it can be detected by fluorescence. The nucleic acid probe may incorporate or be covalently bound to a fluorescent dye, it may be modified with a hapten to allow a fluorescent reagent to bind to the nucleic acid probe, or it may be primarily or secondarily labeled with an enzyme which is detected by the use of a fluorogenic substrate. Hapten/hapten binding polypeptide pairs, useful for detection of nucleic acid probe hybridization, include (but are not limited to) biotin/avidin or streptavidin, digoxigenin/a-digoxigenin antibodies, and dinitrophenol (DNP)/a-DNP antibodies.

At least one nucleic acid probe is used to identify fetal cells in a maternal sample, although the use of at least 2, 3, 4, 5 or more nucleic acid probes is contemplated. When more than one nucleic acid probe is used, each different nucleic acid probe can be detected using a different fluorescent dye, so that cells expressing multiple nucleic acid probes can be identified. In addition to the nucleic acids of the invention, probes corresponding to genes that are preferentially expressed in fetal cells in a maternal blood sample include, but are not limited to, fetal hemoglobin probes, paternal HLA determinant probes, Y chromosome specific probes, With respect to fetal hemoglobin probes, probes to transcripts of the γ-, ε-, or ζ-globin probes can be used, although an ε-globin probe is preferred. Because γ-globin transcripts are expressed in RBCs of adults with hereditary persistence of fetal hemoglobin or σβ thalassemia, and ζ-globin probes transcripts are expressed in RBCs of adults with α thalassemia.

When multiple dyes are used in conjunction with multiple fetal cell probes, it is preferable that the various dyes have non-overlapping fluorescent spectra, or at least that the emissions spectra be distinguishable through the use of narrow pass filters. A large number of fluorescent dyes are known in the art and commercially available. Commonly used fluorescent dyes include fluorescein, rhodamine, texas red, phycoerythrin, Hoechst 33258, Cascade Blue, Cy3, and derivatives thereof. Alternatively, multiple nucleic acids probes can comprise the same type of label for the purpose of improving the signal to noise ratio of a single probe.

Methods for in situ hybridization (ISH) are well known in the art. Because the cells analyzed by the methods of the invention are generally in a suspension, ISH is normally carried out on fixed, permeabilized cells which have been fixed to an insoluble substrate, such as a poly-L-lysine-coated glass slide or polystyrene plate or dish, although ISH may also be carried out on fixed cells in suspension. Where the cells are adhered to a substrate, the substrate is preferably transparent to visible and ultraviolet light (e.g., glass), to allow for use of fluorescent dyes as labels. As will be appreciated by one of skill in the art, materials and solutions used in preparation of cells for ISH and for ISH itself are preferably RNase-free.

Generally, a suspension of cells, preferably at least about 106, 107 or 2×107 cells/milliliter is made in a solution comprising little or no added protein (e.g., serum free medium or a balanced salt solution) and placed on substrate which has been derivatized to allow attachment of cells by use of a crosslinking agent. Preferably, the substrate is modified by coating with poly-L-lysine or by “subbing” with gelatin. The cell suspension is placed on the substrate, generally as a small “pool” or drop on the surface of the substrate, and the cells are allowed to attach to the substrate by settling under normal gravity for a period of time, preferably at least about 10, 20 or 30 minutes, although the cells may be “spun” onto the substrate by the use of a centrifuge with an approprate rotor adapted to hold the substrate. Attachment of the cells onto the substrate is preferably accomplished under conditions of humidity approaching 100%, as will be apparent to one of skill in the art.

After the cells have attached to the substrate, the cells are crosslinked to the substrate (or to the derivative bound to the substrate) using a fixative. Any appropriate fixative may be used, including acid alcohol solutions, acid acetone solutions, aldehyde fixatives, homobifunctional crosslinking agents such as N-hydroxysuccinimide (NHS) esters (e.g., disuccinimidyl suberate, disuccinimidyl glutarate, and the like) and heterobifuncational crosslinking agents known in the art. Preferably, an aldehyde fixative such as formaldehyde, paraformaldehyde or glutaraldehyde, is used to crosslink cells to poly-L-lysine or gelatin coated substrates. Preferably, the cells are fixed to the substrate by placing the substrate with attached cells into a bath of fixative solution, although fixation may be accomplished by replacing the pool or drop of liquid containing the cells with a similar volume of fixative. The attached cells and substrate are incubated in the fixative for a period of time appropriate to the particular fixative selected by the practitioner, preferably about 20 minutes in the case of 4% paraformaldehyde.

After fixation, the substrate may be rinsed, typically with a buffered saline solution such as phosphate buffered saline or tris-buffered saline, dehydrated using a series of ethanol baths (e.g., by incubating the fixed cells in 50%, 70%, 95%, and 100% ethanol for 2-5 minutes each) air dried, and stored for later ISH procesing. Where the cell/substrate preparation is intended for immediate ISH processing, the cells must still be permeabilized, preferably by incubating the cell/substrate preparation in 50% ethanol, although detergent solutions, such as 0.01 to 0.1% t-octylphenoxypolyethoxyethanol or polyoxyethylenesorbitan monolaurate, may also be used.

Alternatively, the cells may be fixed in solution using an appropriate fixative, rinsed, dehydrated and embedded in paraffin, then sectioned and adhered to glass slides using conventional histologic processing techniques. Prior to processing for ISH, cells processed in this matter must be de-paraffinized, typically by use of a xylene bath, and rehydrated by processing through progressively less concentrated ethanol solutions, as is well known in the art.

The cells to be analyzed are first denatured, generally by use of extreme pH (e.g., 0.2 N HCl for 10-30 minutes at room temperature) followed by high temperature (e.g., 10-20 minutes at 70° C. in 2×SSC), and an additional digestion with a non-specific protease (e.g., pronase) may be included as well. After denaturation, a post-fixation step is preferably performed by incubating the denatured cells in fixative (e.g., five minutes in 4% paraformaldehyde at room temperature), followed by rinsing in a buffered salt solution.

Non-specific binding sites on the cell/substrate preparation are preferably blocked prior to hybridization with probes, typically by acetylation and modification of free sulfur groups. Preferably such blocking is carried out by incubating the cell/substrate preparation in a sulfur reducing agent (e.g., 10 mM dithiothreitol, DTT, in buffered saline at elevated temperature, such as 10 minutes at 45° C.), followed by incubation with DTT, iodoacetamide, and N-ethylmaleimide (e.g., 10 mM DTT, 10 mM iodoacetamide, 10 mM N-ethylmaleimide for 30 minutes at 45° C.). Additional blocking of polar and charged groups may be accomplished by incubation of the cell/substrate preparation in acetic anhydride (e.g., 0.25 to 0.5% for 5-10 minutes at room temperature).

Probe nucleic acid probe is denatured prior to hybridization with the prepared cells. Normally, the probe is precipitated in ethanol, then redissolved in a small volume of solvent such as 2×SSC, 1×TEA, or formamide, heat denatured by incubating at 70° C. or higher for 10-20 minutes, then added to a hybridization mixture. A non-specific, unlabeled DNA, such as sonicated salmon sperm DNA is preferably denatured along with the probe. Generally, when more than one probe is used, the probes are hybridized with the cells at the same time, although use of multiple probes does require use of divergent labeling systems to avoid signal crossover.

Hybridization is typically carried out at elevated temperature in hybridization mix containing a buffered salt solution (e.g., 4×SSC), a high molecular weight polymer to increase the effective concentration of the probe(s) (e.g., 20% dextran sulfate), and a protein blocking agent (e.g., 2 mg/mL high purity bovine serum albumin). Hybridization is typically carried out under a coverslip which may be anchored in place with rubber cement or any other material which serves to temporarily anchor the coverslip and reduce evaporation of the hybridization mixture. Hybridization is preferably carried out under conditions where the hybridization temperature is 12-20° C. below the melting temperature (Tm) of the probe. The Tm of a long nucleic acid can be found as Tₘ=81.5−16.6(log10[Na+])+0.41(%G+C)−0.63(%formamide)−600/N, where N=the length of the selectively hybridizable nucleic acid under study, while the Tm of oligonucleotides from about 70 to 15 nucleotides in length may be found as Tₘ=81.5−16.6(log10[Na+])+0.41(%G+C)−600/N, and the Tm of short oligonucleotides of <14 nucleotides may be found as Tm=2(A+T)+4(G+C), where A, T, G and C are the numbers of adenosine, thymidine, guanosine and cytosine residues, respectively. Hybridization may be accomplished in as short a period as 2-4 hours, although longer hybridization incubations are also acceptable. Alternatively, glycerol-based ISH technology, such as that disclosed in International Patent Application No. WO 96/31626 or U.S. Pat. No. 5,948,617, may be used.

After the hybridization incubation is completed, the hybridization solution is removed, and the cells are washed, typically for 15 minutes each in 50% formamide/2×SSC at 37° C., 2×SSC at 37° C., and 1×SSC at room temperature. After washing is completed, the cells are incubated in the detection reagent (e.g., fluorescently-labeled avidin or streptavidin for a biotinylated probe). The exact conditions of the incubation with the detection reagent will vary depending on the exact identity of the detection reagent, but is typically accomplished by incubation for 30-60 minutes at 37° C. in a chamber protected from ambient light (to reduce photobleaching of the fluorescent label), although signal amplification techniques generally require multiple incubations, as will be apparent to one of skill in the art. Amplification techniques such as the use of secondary antibodies which bind to a primary detection reagent or enzymatic amplification may be employed if so desired. Excess detection or amplification reagent is washed away, typically by rinsing with a buffered salt solution (e.g., 4×SSC) at room temperature. Optionally, a rinse including a detergent (e.g., 0.1% t-octylphenoxypolyethoxyethanol) in the buffered salt solution may be incorporated in the wash protocol.

Genomic DNA in the cells may be counterstained by incubation with a double-stranded DNA-binding dye, such as propidium iodide or 4,6-diamidino-2-phenylindole (DAPI) and rinsing away unbound dye.

Where immunoenriched cells are processed as cells in suspension, the cells are carried through a substantially similar process, except that the cells are collected by centrifugation or filtration after each step (e.g., after fixation, each wash step, etc.).

After hybridization, labeling with detection reagent and counterstaining, the cells are preferably sealed under a coverslip with an anti-fading reagent appropriate to the fluorescent dye(s) used in the detection reagent. The appropriate anti-fading reagent can be easily selected by the skilled practitioner.

Fetal cells may be detected by the use of any convenient fluorescent microscopy technique, including epifluorescence microscopy, confocal fluorescence microscopy, and other techniques known in the art. Results of microscopy may be stored on photographic negatives, photographic plates, or on magnetic or optical storage media when a CCD camera or other electronic imaging equipment is used. Alternatively, cells which are processed as cells in suspension may be analyzed using FACS technology.

Nucleated cells which are present in the sample following immunoenrichment and are labeled by at least one of the nucleic acid probes are considered identified as fetal cells.

In situ single cell PCR, for example using PCR primers corresponding to the nucleic acids of the invention, also offers a method for detection of single cells of fetal origin. With this method, each cell, fixed either in suspension or on a solid support, and either as a single cell or in the context of surrounding tissue, functions individually as a reaction chamber for the PCR. With proper fixation and permeabilization conditions, the oligonucleotide primers and other reaction components are able to diffuse into the cells, and, upon thermal cycling, are able to amplify available specific target sequences. The product DNA retained within the source cell can be readily detected by standard in situ hybridization (Brezinschek et al., 1995, J. Immunol. 155:190). For diagnostic purposes, single fetal cells can be isolated and product DNA can alternatively be extracted and subjected to gel electrophoresis or southern blotting.

Specific or selective acting fetal cell probes of the invention can be labeled with radioactive labels including radionucleides (e.g. ³⁵S, ³²P or ³H) and hybridized to nucleic acid that has been extracted or amplified via various PCR techniques from single cells or samples of cells. Southern and Northern blot analyses standard for practitioners in the art can then be utilized to confirm presence of fetal cells in nucleic acid extracted from the samples. If RT-PCR is used in conjunction with the fetal cell associated primers of the invention to produce cDNA's specific to fetal cells in a mixed fetal maternal sample, then detection of amplified cDNA in cells could also be accomplished with radioactive labeling of cDNA followed by autoradiography or scintillation counting.

In a preferred embodiment, non-isotopic labels (e.g. biotin or digoxigenin) for RNA probes could ideally be used for non-radioactive in situ and Northern blotting applications to detect fetal cells. Non-isotopic labeled RNA probes offer several advantages over other types of probes: RNA/RNA hybrids are more stable than RNA/DNA hybrids; RNA probes are single stranded and don't re-anneal on themselves; RNA probes can be labeled throughout the molecule; and RNase A can be used to eliminate unhybridized single stranded probe. These factors result in RNA probes that are more sensitive and have lower background than either cDNA or oligonucleotide probes. Thus, the fetal cell probes of the invention, labeled with non-isotopic identifiers offer a superior technique for detection. In addition the fetal cell probes of the invention, with non-isotopic labels, enable simultaneous use of probes specific for genetic disorders or traits and aimed at nuclear DNA.

If the non-isotopic labeled RNA probes contain fluorescence markers, then fetal cells may be detected by the use of any convenient fluorescent microscopy technique, including epifluorescence microscopy, confocal fluorescence microscopy, and other techniques known in the art. Results of microscopy may be stored on photographic negatives, photographic plates, or on magnetic or optical storage media when a CCD camera or other electronic imaging equipment is used. Alternatively, cells which are processed as cells in suspension may be analyzed using FACS technology.

Identification of fetal cells with nucleic acid probes is normally carried out using an in situ approach, generally fluorescent in situ hybridization (FISH), although in situ amplification methods are also contemplated, as discussed above. For FISH, a nucleic acid probe is modified (or synthesized with modified nucleotides) so that it can be detected by fluorescence.

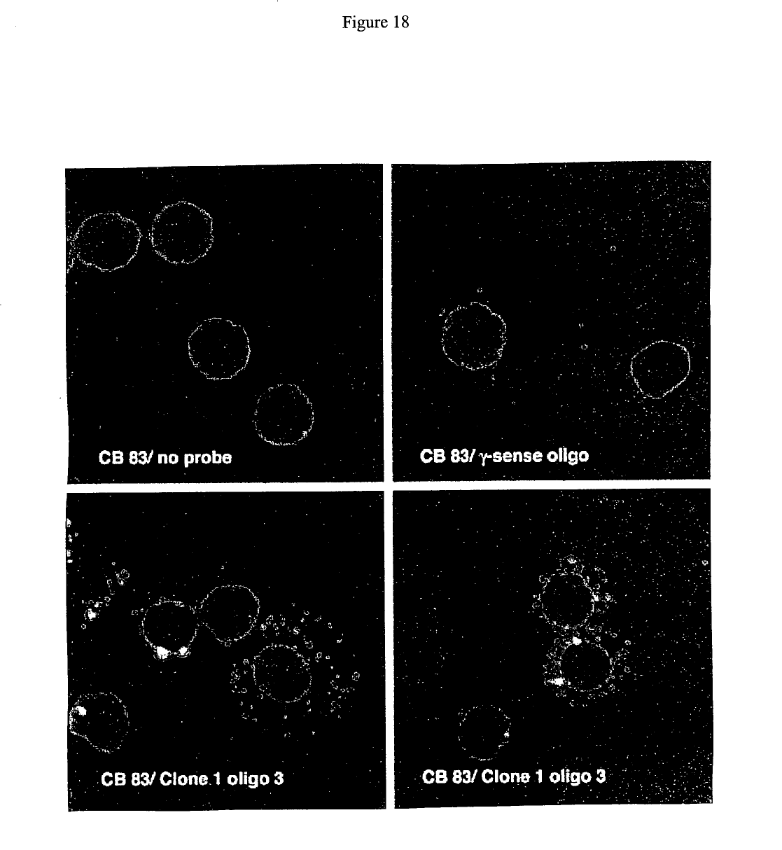

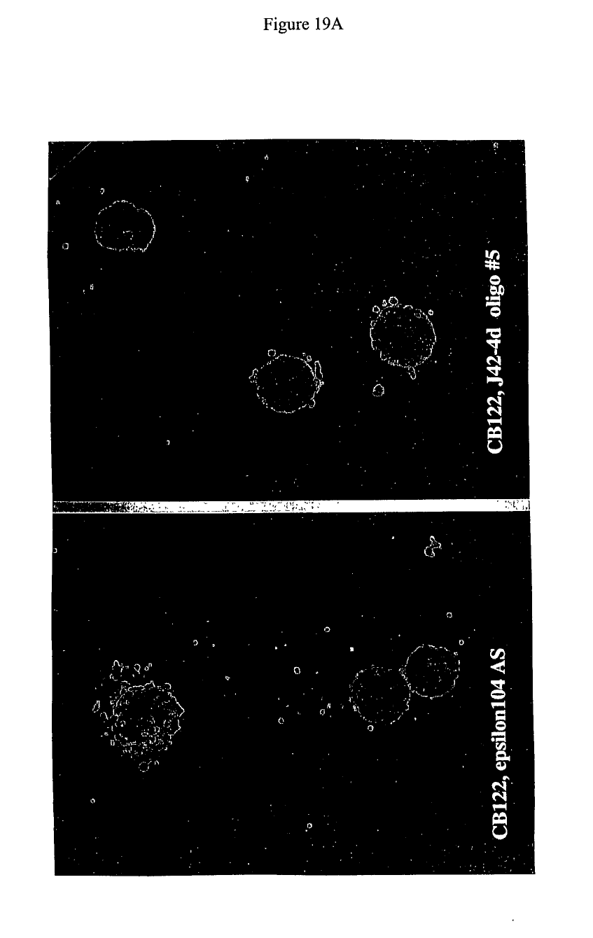

A fetal cell probe is a reagent for detecting a fetal cell RNA contained in a fetal cell potentially present in a sample of interest by a hybridization reaction. Usually, a probe will comprise a label or a means by which a label can be attached, either before or subsequent to the hybridization reaction. Means for attaching labels include biotin moieties that couple with avidin or streptavidin, haptens that couple with anti-hapten antibody, and particular nucleic acid sequences (optionally on a branch or fork) that hybridize with a reagent nucleic acid having a complementary sequence, any of which ultimately lead to the attachment of a label. Suitable labels include radioisotopes, fluorochromes, chemiluminescent compounds, dyes, and proteins, including enzymes. The probe may incorporate or be covalently bound to a fluorescent dye, it may be modified with a hapten to allow a fluorescent reagent to bind to the probe, or it may be primarily or secondarily labeled with an enzyme which is detected by the use of a fluorogenic substrate. Hapten/hapten binding protein pairs, useful for detection of nucleic acid specifier hybridization, include (but are not limited to) biotin/avidin or streptavidin, digoxigenin/a-digoxigenin antibodies, and dinitrophenol (DNP)/a-DNP antibodies. In preferred embodiment, the signal arising from the probe which indicates hybrid formation between a probe and its target is described in FIG. 18 and Section 8, infra. Such modifications are also contemplated for the diagnostic probes that are used in conjunction with the fetal cell probes of the invention.

In other embodiment of the present invention, instead of, or in conjunction with, using fetal cell associated probes to identify rare fetal cells in a maternal blood sample, an antibody that immunospecifically binds to a fetal cell antigen, such as an antibody directed against a polypeptide of the invention or an antibody that is identified by the methods described in Section 5.7.2, supra, including but not limited to the anti-Clone-1 antibody or derivatives thereof, can be used for fetal cell detection. A maternal blood sample, which has been optionally immunoenriched for fetal cell, is contacted by an antibody against a fetal cell associated antigen, including but not limited to the antibodies described of Sections 5.5 and 5.7.2. Antibody-bound cells can be identified by routine immunostaining methods known in the art. As will be readily apparent to one of skill in the art, the signal amplification step of FIG. 18 and Section 8 can be readily adapted to methods where the agent bound to fetal cells is an antibody rather than a nucleic acid probe.

### 5.9. Methods of Fetal Cell Diagnosis

The identification methods of the present invention allow for non-invasive prenatal diagnostics. As discussed above, in a preferred embodiment, the identification of fetal cells involves contacting the biological sample containing the cell or a cell extract with the probe under conditions where the nucleic acid can selectively or specifically hybridize with the target transcript. The target transcript may be RNA or a cDNA copy. Where a cell extract is used, formation of a stable hybrid will indicate that at least one cell containing the transcript was present in the original cell population. Where permeabilized whole cells are used, detection of hybrid formation will indicate which cells in the population contain the transcript.

Optionally, the fetal cells are separated from the maternal cells prior to carrying out the diagnostic methods of the invention. Thus, the probe sequences can also be used to separate fetal cells expressing the target transcript from maternal cells in a mixed population. An example of an intracytoplasmic staining method for cell separation using nucleic acid sequences is described generally in U.S. Pat. No. 5,648,220. Briefly, the cell is lightly fixed with 2-8% paraformaldehyde and penneabilized with aqueous alcohol, such that the cell remains sufficiently intact to retain the target sequence. The cells are then contacted with a probe sequence or plurality of sequences to which a detectable label (such as a fluorescence marker) is attached. After washing, the identified cells are then separated from other cells, either by micromanipulation, or by an automated method such as fluorescence-activated cell sorting.

Where the detection and diagnostic methods of the invention entail the use of PCR, the PCR reaction can be performed in situ. For the diagnostic methods of the invention, the PCR reaction can be performed on a single cell that has been identified by the fetal cell probes and antibodies of the invention to be a fetal cell. Micromanipulation methods are known in the art and can be used to separate a fetal cell from the maternal blood sample and place into a suitable container for the PCR reaction.

Identified fetal cells from a maternal sample can be used in diagnostic assays, particularly assays for genetic diseases, as will be apparent to one of skill in the art. Normally, such diagnostic assays are carried out using FISH technology and a diagnostic probe. As will be apparent to one of skill in the art, diagnostics assays on the fetal cells may be carried out after the ISH procedure with the fetal cell probe or antibody, or may be carried out concurrently. The exact size and sequence of the diagnostic probe will depend on the identity of the genetic disorder which is the subject of testing. For example, when testing for a trisomy (e.g., Down's Syndrome or trisomy 21), a probe specific for the chromosome of interest is utilized, while testing for genetic diseases will utilize one or more probes specific for disease-causing or associated alleles. In a preferred embodiment, the trisomy 21 probe is the AneuVysion® probe (Vysis).

When the diagnostic assay is carried out sequentially (e.g., after identification of fetal cells with a probe DNA), the location of fetal cells in the sample can be recorded, then the DNA probes and detection reagents can be removed from the sample by stripping. Generally, stripping is accomplished by denaturing the sample using extreme pH or elevated temperatures. After denaturation, the sample is processed using the desired diagnostic assay. As will be apparent to one of skill in the art, the details of conducting the assay will depend on the exact identity of the assay and the form of the sample.

As will be apparent to one of skill in the art, diagnostic assays carried out concurrently with the DNA probe ISH step should be assays which do not interfere with the DNA probe and detection system utilized. Accordingly, a diagnostic assay run concurrently with the specification step will normally utilize a non-overlapping detection system (e.g., where the DNA probe step utilizes a biotinylated probe, the diagnostic assay utilizes a different detection technology, such as digoxigenin-modified probes, and the fluorescent dyes utilized in the detection system will be different). However, the same detection system may be used if the subcellular localization of the fetal cell vs. diagnostic probe (e.g.,

A wide variety of diagnostic assay technologies and probes are available for detection of chromosomal abnormalities and/or genetic diseases. For example, U.S. Pat. No. 5,447,841 discloses probes specific for chromosome 21, which may be utilized in a diagnostic assay for trisomy 21 (i.e., Down's syndrome). Multiple genetic disorders may be assayed in a single test utilizing the multiplex FISH methods disclosed in U.S. Pat. No. 6,007,994.

### 5.9.1. Diagnosis of Fetal Genetic Abnormalities

Once the fetal cell probes of the invention have been employed to identify fetal nRBC in maternal blood samples, several possibilities emerge for diagnosis and genotyping of genetic disorders. Fluorescence DNA probes specific to interphase stage nuclei have been developed to identify chromosomal disorders of aneuploidy (Down syndrome, Klinefelter syndrome, and trisomy 13) (Simpson and Elias, 1995, Human Reproductive Upate 1(4):409-418). FISH analysis (fluorescence in situ hybridization) using dual color X and Y specific DNA probes has also been developed to determine fetal sex. The advantage of such techniques is that these nuclear DNA probes can be used simultaneously with the RNA tag based probes of this invention, allowing for a non-laborious method of multiple diagnoses through multiprobe florescence in situ hybridization.

If identified fetal cells have been sufficiently isolated from maternal cells, for example by single cell micromanipulation techniques and PCR described below, then mutation detection by fetal DNA analysis can be conducted. The genes responsible for many single gene disorders have been mapped and cloned, including spina bifida, sickle-cell anemia, thalassaemias, Marfan Syndrome, and Duchenne Muscular Dystrophy. For the single gene mutation causing Cystic Fibrosis, PCR or ARMS multiplex tests are typically used to detect the known causal mutations (Ferrie et al., 1992, Am. J. Hum. Genet. 51(2): 251-262). Amplification proceeds with PCR primers specific to known mutations in the gene. A diagnosis can be made which is then confirmed by DNA sequence analysis of the gene.

Another category of single gene disorders encompassed by the diagnosis methods of the present invention relates to diseases caused by expansion of blocks of repeating nucleotide triplets within a gene. For many of these disorders, PCR based primers have been developed for the responsible genes, including Fragile X syndrome (FMR1 gene)(Chong et al., 1994 Am J Med Genet 51:522-526), Friedreich's ataxia (Filla et al, 1996, Am J Hum Genet 59:554-560), myotonic dystrophy (Brook et al., 1992, Cell 21;68(4):799-808.), and Huntington's Disease (Warner et al., 1993, Mol Cell Probes 7: 235-139). In Huntington disease, the DNA sequence, CAG, is part of this sequence. This sequence may be duplicated many times in individuals, up to 26 times in the general population. The duplication of this segment is called a “trinucleotide repeat” in which these three nucleotides (CAG pattern) are repeated over and over again. Individuals with Huntington disease may have from 40 to over 100 repeated CAG segments. The normal number of CAG repeats is from 11-24. Since the sizing of alleles is essential to diagnosis, DNA sequencing is performed. Southern blotting is also used to back up the PCR test, especially for large amplifications or individuals with a single normal allele. The sequences labeled and used as probes for lengths of repeats could serve as a basis for developing probes which could be utilized in situ with fetal nRBC's. Such nuclear DNA probes could be used simultaneously with the RNA cytoplasmic probes based on the tag sequences of this invention. Thus allowing for detection, by florescent microscopy screening, of individual fetal cells with genetic disorder markers. The DNA specific probes and associated assays would not interfere with the RNA-based fetal cell detection system, providing and added benefit. The present invention might also allow for diagnosis of fetal infections, including but not limited to retroviral infections (e.g., HIV). The fetal cell genome can be diagnosed by contacting the fetal cell identified by the methods of the invention (before or after identification) with a probe that will hybridize to genomes of infectious agents of interest.

Certain techniques for acquiring genetic information, especially pertaining to human genetic disorders can be used following or before detection of target cells using fetal cell probes of the invention, or simultaneously with the fetal cell probes or fetal cell antibodies of the invention. Used in combination with available genetic diagnostic procedures, the fetal cell probes and antibodies of the invention aid in detection and confirmation of genetic disorders of a developing fetus. Once the fetal cell probes and antibodies of the invention have been employed to identify fetal nRBC's in maternal blood samples, several possibilities emerge as techniques which can be utilized for diagnosis and genotyping of genetic disorders and traits in the fetus.

### 5.9.2. Diagnosis of Other Fetal Characteristics

Fluorescence probes specific for certain fetal characteristics exist and can be simultaneously or successively utilized with the instant invention. For example, the sex of a fetus is commonly desired knowledge. FISH analysis (fluorescence in situ hybridization) using dual color X and Y specific DNA probes has been developed to determine fetal sex. The advantage of such techniques is that these nuclear DNA probes can be used simultaneously with the fetal cell probes and antibodies of the invention, allowing for a non-laborious method of multiple diagnoses through multiprobe florescence in situ hybridization. Fluorescence probes specific to Y chromosome (i.e. the Vysis® LSI SRY DNA FISH probes or the Vysis® WCP Y DNA DNA FISH probe) are targeted at nuclear genetic material not cytoplasmic and thus do not interfere with the fetal cell probes of the invention. Fetal cell probes designed for specific or selective markers in the fetal cell could be utilized, since the maternal genome does not contain Y specific genes. Probes specific to the X chromosome exist (i.e. the Vysis® CEP X probes) as well and could be used in a similar manner to determine sex of the fetus if such probes were used in conjunction with fetal cell probes of the invention which in this case must be targeted at specific markers of the fetal cells.

PCR offers an alternative method to hybridization for determination of sex. PCR primers specific to genes exclusive to the Y chromosome can also be utilized in conjunction with the fetal cell probes of the invention to determine fetal sex. The PCR reactions may precede, succeed, or occur simultaneous with the fetal probe hybridization technique or use of fetal antibodies of the invention.

Allele-specific PCR primers for the alleles of genes encoding for the proteins responsible for blood types exist. Primers specific to the RhD gene responsible for Rh factor (Gassner et al., 1997, Transfusion 37:1020) are a good example. Such primers can provide genotype data that can be used to determine blood type of the fetus. The fetal cell probes of the invention can provide some degree of confirmation of diagnosis based on PCR results and in conjunction with the PCR can provide exact data necessary to determine the genotype of a fetus with respect to the RhD gene alleles provided the fetal probes of the invention are developed based on specific markers. The PCR reactions may precede, succeed, or occur simultaneous with the fetal cell probe hybridization technique or use of the fetal cell antibodies of the invention.

In cases of multiple pregnancy, the fetal cell probes or antibodies of the invention could be combined with single cell isolation and standard DNA fingerprinting techniques to detected the presence of multiple fetuses at early stages of pregnancy, provided the fetuses are not genetically identical.

With detection of target cells using the fetal cell probes of the invention, any human trait for which the gene(s) the trait controls have been identified can be examined, provided probes or PCR primers specific to alleles responsible for the trait of interest have been developed. The fetal cell probes and the fetal cell antibodies of the invention when utilized in conjunction with existing and future molecular diagnostic techniques will result in an increase of potentially valuable fetal genetic information available to physicians during gestation and after birth.

### 5.9.3. In situ Detection of Genetic Abnormalities

In situ fetal diagnoses of genetic abnormalities can be achieved by combining the fetal cell probes or antibodies of the invention with existing probes aimed at nuclear genetic material that enable one to determine the number of chromosome copies present in fetal cells. Flow cytometry followed by the use of one or more of the various Vysis® DNA FISH probes specific to human chromosomes or portions thereof or other such probes specific to human chromosomes enables detection of fetal aneuploidy (Down syndrome, Klinefelter syndrome, and trisomy 13). Fluorescence DNA probes specific to interphase stage nuclei have been developed to identify chromosomal disorders of aneuploidy (Simpson and Elias, 1995, Human Reproductive Upate 1(4):409-418). If a sample has been enriched for fetal cells, e.g., using an antibody of the invention, it may not necessary to identify fetal cells in the situation as described above, because maternal cells lack such abnormalities. However, the cytoplasmic fetal cell probes of the invention, aimed at either specific or selective fetal cell markers, whether used before, after, or at the same time as other diagnostic techniques provide and additional confirmation that the diagnosis is limited to the fetal genome. The fetal cell probes and antibodies of the invention, used in conjunction with probes such as those mentioned above, would reduce the number of cells screened in a sample of mixed fetal/maternal blood, reducing the time necessary for diagnosis procedures. In addition the RNA-based cytoplasm specific fetal cell probes do not interfere with the procedures for the nuclear-based probes mentioned above.

Fluorescence probes for microdeletion syndromes also have directed to nuclear DNA that can with easily be utilized in conjunction with the fetal cell probes and antibodies of the invention. Probes for microdeletion syndromes such as DiGeorge, Velocardiofacial, Cri Du Chat, Miller-Dieker, LSI Prader-Willi/Angelman, Smith-Magenis, Wolf-Hirschhorn, and LSI Steroid Sulfatase can be synthesized or purchased. Again, the hybridization probes specific to genetic material responsible for these disorders may be used preceding, succeeding, or simultaneously with the fetal probe hybridization technique or use of fetal antibodies of the invention.

Similarly, the fetal cell probes and antibodies of the invention can be used in conjunction with the other fluorescence probes that enable detection of abnormalities of chromosome telomeric regions, chromosome rearrangements, chromosome deletions & additions, and chromosome translocations. Such probes are commercially available, for example from Vysis. As the availability and number of probes for genetic traits and disorders increases so will the utility of the fetal cell probes and antibodies of the invention. Combined, the probes provide a means to increase the specificity and speed with which a diagnosis can be made.

### 5.9.4. Detection of Genetic Abnormalities by PCR

For single gene genetic disorders where the mother has been genotyped as a carrier, the fetus may have the same genotype with respect to gene responsible for disorders. Techniques such as those described in the previous section are insufficient in this situation and the fetal cell probes and antibodies of the invention then have added value and necessity, since diagnosis cannot generally be made from maternal or enriched fetal cell blood samples in the absence of methods for identifying the fetal target cells with specificity. PCR techniques utilized in connection with the fetal cell probes and antibodies of the invention provide a means to diagnose genotypes in situations where both the mother and fetus are carriers.

The use of PCR techniques in detection of genetic abnormalities can be done in situ or following DNA extraction from single identified fetal cells. The efficiency of methods for DNA extraction from single cells is continually being improved http://www.aps.org/meet/MAR01/baps/abs/S2730006.html (Findlay et al., 1997, Nature 389:555-556; Ray and Handyside, 1996, Mol. Hum. Reprod, 2:213-218). These techniques can utilized with isolated fetal cells. Another option is to conduct PCR without any extraction procedure (Küppers et al., 1997, Handbook of Exp. Immunol. 5th ed., Eds. D. M. Weir et al., Blackwell Scientific). With this method, each cell, fixed either in suspension or on a solid support, and either as a single cell or in the context of surrounding tissue, functions individually as a reaction chamber for the PCR. With proper fixation and permeabilization conditions, the oligonucleotide primers and other reaction components are able to diffuse into the cells, and, upon thermal cycling, are able to amplify available specific target sequences. Product DNA is retained within the source cell and is readily detectable by standard in situ hybridization. (Brezinschek et al.,1995, J. Immunol. 155, 190). Nucleic acid sequences can now be amplified within the environment of the cell (Komminoth et al, 1992, Diagn. Mol. Pathol. 1:85; Nuovo et al., 1991, Am. J. Pathol. 139:1239). In situ PCR can be performed on a single fetal blood cell samples allowing for detection of genetic disorders for which specific primers have been designed. By incorporating molecular beacons into the PCR reaction, a fluorescence can be observed in cells possessing mutated gene copies responsible for the genetic disorder being tested. (Pierce et al., 2000, Molec Human Reproduction, 6(12):1155-1164; Giesendorf et al., 1998, Clin Chem 44:482-486; Bonnet et al.,1999, Proc Natl Acad Sci USA 96:6171-6176; Kostrikis et al., 1998, Science, 279:1228-1229). In addition, single cell PCR has been successfully used to identify heterozygous loci http://www.promega.com/geneticidproc/eusvmp2proc/21.pdf. Such techniques could be used to determine if a fetus is a carrier for the genetic disorder being tested.

For fetal cells identified the cytoplasm specific fetal cell probes, further isolation from maternal cells in a sample can be achieved by several methods known to those of skill in the art. Techniques for single cell micromanipulation have been developed for embryo manipulation in preimplantation genetic diagnosis and fertility treatments and have successfully been applied to other cell types (Leary, 1994. In: Methods in Cell Biology. Flow Cytometry, Darzynkiewicz et al., eds. vol. 42:pp. 331-358; Iritani, 1991, Mol. Reprod. Dev., 28:199-207). The essential equipment consists of an inverted phase fluorescence microscope suitable for observation of single cells that has been fitted with a Narishige micromanipulation/microinjection system for single cell manipulation. The manipulation is dependent on drawn glass capillaries. An alternative method, using a similar microscope, employs an optical trapping system (lazer tweezers). In this technique a laser beam is capable of catching and holding both static and motile cells (Moravcik Z. et al. 1998. The Journal of Eukaryotic Microbiology conference procedings). Grover has had success in using an optical trapping system with erythrocytes (Grover et al., 2000, journal of Optical Society of America 7(13):533). Thus to isolate a single tag-labeled fetal cell in a sample of maternal blood cells can be accomplished either by using micromanipulation or an optical trapping system.

In situations where the maternal genotype may have copies of genes responsible for the genetic disorders being tested for in the fetus, the fetal cell probes and antibodies of the invention designed to specific markers are required and isolation of target cells may be necessary if DNA extraction is required. The isolation may be mechanical, followed by single cell PCR, or visual utilizing fluorescence microscopy. For example, if PCR primers specific to copies of genes responsible for the trait or disorder being tested for are used in connection with fluorescence markers such as molecular beacons, then the PCR reaction could be conducted before the use of fetal cell probes and cells with both cytoplsamic fluorescence and nuclear (preferably of differing colors) could be identified. If specific nuclear PCR primers have been developed for both normal and mutated copies of disease genes or for mutant copies of genes with intermediate expression, then multi-colored probes could be employed to identify single fetal cells and determine the carrier status of the fetus or the likely severity of the disorder based on genetic compliment the fetus has inherited. The possibility that the fetal cell probes and antibodies of the invention could be used before, after, or simultaneously with such PCR gene specific primers makes the combined use of technologies a strong one.

If identified fetal cells have been sufficiently isolated form maternal cells my methods described in the present application, then mutation detection by fetal DNA analysis can be conducted. Allele-specific PCR primers for alleles of the RhD gene (Gassner et al., 1997, Transfusion 37:1020) which determines human Rh factor have potential in diagnosing the possibility of erythroblastosis fetalis when utilized in connection with the fetal cell probes and antibodies of the invention. Additional single gene disorders or fetal genetic characteristics, e.g., gender or infection, that can be diagnosed by PCR-based techniques are described in Sections 5.9.1 and 5.9.2, supra. Amplification proceeds with PCR primers specific to known mutations in the gene. A diagnosis can be made which is then confirmed by DNA sequence analysis of the gene.

### 5.9.5. Methods for Simultaneous RNA and Genomic DNA Hybridization

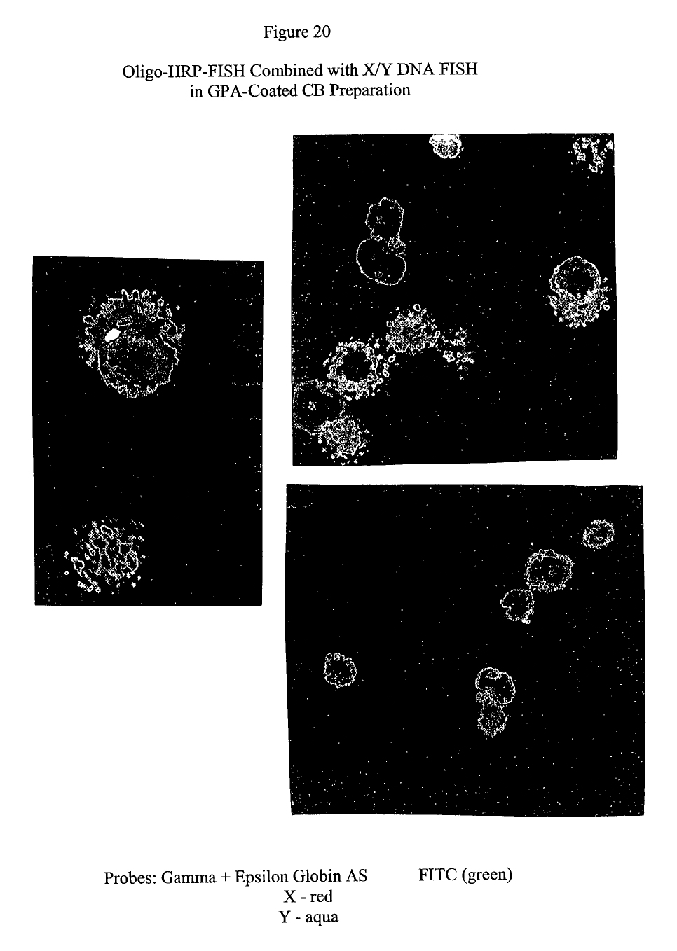

As discussed above, in a preferred embodiment of the invention, the fetal cell probes of the invention are used simultaneously with one or multiple fluorescence probes designed to detect specific chromosomes or genomic markers, for example for diagnosis of genetic disorders. The present inventors have developed techniques that allow for the first time simultaneous hybridization with a probe directed to a cytoplasmic RNA and a probe directed to a nuclear DNA. These technique maintain the best possible cell/sample conservation, a strong distinguishable signal in the highest number of cells, eliminate of background autoflourescence, and allow detection of the highest possible fetal cell number in the maternal cell sample. The simultaneous hybridization techniques of the invention entail the use of one, preferably more than one, preferably at least three or four of the following features. These techniques have been used successfully to detect X and Y chromosome sequences (using probes purchased from Vysis) in the nuclei and epsilon and gamma globulin RNAs in fetal cord blood cells (see FIG. 20)

One feature entails, prior to hybridization, coating slides with anti-cell-surface antibodies, for example anti-IgG antibodies (GPA), followed by the addition of cell suspension and centrifugation. This enables more cells to remain intact, minimizing cell loss.

Another feature entails using probes for both RNA and genomic DNA that comprise at least 45%, most preferably at least 50% GC content. The probes are preferably 25-300, most preferably 30-200, e.g., approximately 30, 50, 70, 100, 150 or 200 nucleotides in length.

Probe accessability can be improved by addition of the detergent Tween-20/PSB (0.2%) incubation at RT for 20 min. and/or addition of the protease Proteinase-K at 0.1 μg/ml incubation for 10 min at RT.

Probe hybridization is preferably carried out for a period of 1.5-4 hours at 40-50° C., most preferably for approximately 2.5 hours at 45° C.

The two most preferable features for inclusion in the simultaneous hybridization techniques relate to fixation. A 4% neutral Formalin/PBS, at a neutral pH of 6 to 8, more preferably at a pH of 6.5-7.5, most preferably at a pH approximately 7, allows fixation of the freshly prepared cell samples. The cell samples are incubated with the fixative for approximately 20 min at room temperature. Following fixation and addition of probes, preferably after the cells are washed to remove probe, a post-fixation step is preferably performed. Post-fixation entails using a 4% Formalin/PBS for approximately 5 minutes at room temperature. The post-fixative is preferably at an acidic pH, for example at a pH of approximately 2, 2.5, 3, 3.5, 4, 4.5, 5 or 5.5.

Following the FISH procedure, samples can be dehydrated and stored at ˜20° C. to aid in retaining long term cell and probe integrity.

### 5.10. Kits

The present invention yet further provides kits comprising in one or more containers a first probe which is a fetal cell of the invention, as described in Section 5.3, supra, or an antibody against a fetal cell associated polypeptide, as described in Sections 5.5 and 5.7.2, supra. The kits of the invention further instructions for diagnostic use and/or a label indicating regulatory approval for diagnostic use. The kits can further comprise one or more antibodies for immunoenriching for fetal cells in a maternal blood sample, for example an antibody that selectively or specifically binds to fetal cells in a maternal blood sample. The kits can also optional comprise a second fetal cell probe, including but not limited to a fetal cell probe of the invention or a fetal globulin probe. The second probe can correspond to the same or a different fetal cell mRNA as the first probe. The kits of the invention can further include diagnostic reagents for determining the gender of the fetal cells or for identifying abnormalities associated with the fetal cells.

The probes and antibodies contained in the kits of the invention are preferably labeled, for example by a radioactive or fluorescent label, a calorimetric reagent, or an enzyme. Optionally, a kit of the invention further comprises reagents for calorimetric detection of the labeled probes and antibodies.

### 6. EXAMPLE

### Identification of Fetal Cell Associated Antibodies

### 6.1. Example 1

### Cell Preparations

Human fetal livers were harvested from terminated pregnancies for use in antibody selection and validation. For the first round of antibody selection, the liver cells were obtained at between 8-26 (optimally at about 10-18) weeks of gestation. Wherever possible, the individual liver was placed immediately on ice after dissection. Optimally, the warm ischemia time is less than 15 min. The liver was gently divided into small pieces, and then the pieces were disaggregated into individual cells between microscope slides. The preparation was then centrifuged gently in phosphate buffered saline (PBS) at 3000 rpm to remove liver parenchymal cells. On some occasions, the fetal erythroblasts were characterized by flow cytometry, using mouse anti-CD36 and anti-Glycophorin A (Edelman et al.). The antibodies were labeled with fluorescein and phycoerythrin respectively, and used according to the directions of the flow cytometer manufacturer (Coulter). Typically, 80-90% of the cells were positively stained.

In some of the phage-display antibody selection experiments, fetal erythroblasts were used that had been treated with papain to improve exposure of antigen. Papain digestion was performed by adding several milligrams of the enzyme to the resuspended erythroblast fraction of a single fetal liver preparation and incubating for several hours at 37° C. The treated cells were then washed and used for antibody selection. Some of the antibody validation experiments were performed using cultured erythroblasts. Umbilical cord blood was obtained after delivery of human newborns. The blood was diluted 1:1 with alpha medium (Alpha MEM, Sigma) containing 2% fetal bovine serum (FBS, PAA Laboratories). The cells were layered onto an equal volume of Histopaque™ 1083 (Sigma) and centrifuged at 390 g for 30 min at 18° C. to obtain mononuclear cells. The culture method used was a modification of Weinberg et al. (Blood 81:2591, 1993). Briefly, cells were cultured in alpha medium containing 30% FBS, 1% BSA (Boehringer-Mannheim fraction V), 100 μM β-mercaptoethanol (Sigma), 50 μg/mL Gentamicin (R and D systems), 10 ng/mL IL-6 (R and D systems), 1.3 U/mL Erythropoietin (Boehringer-Mannheim), and 1 mM L-glutamine (Sigma). Cultures were inoculated with 1-2×10⁷ cells into 10 mL of culture medium, and incubated for 10-21 days at 37° C., 5% CO₂. At day 15, the majority of cells in culture are erythroid, and express high levels of CD36 with a range of Glycoprotein A expression.

For the generation or validation of assays using trophoblast markers, syncytiotrophoblasts are obtained as follows: First trimester placentas are obtained from apparently healthy pregnancies electively terminated by aspiration at 6-10 weeks gestation. Clotted blood and any adherent decidua are carefully dissected from the placentas. Syncytiotrophoblasts are isolated by gently teasing the placentas through a 250-mesh sieve. The sheets of syncytiotrophoblast, being significantly larger than contaminating cells, readily sediment at unit gravity in isotonic medium. After sedimentation for approximately 2 min, the supernatant is decanted and the cells resuspended in fresh solution. This washing procedure is performed three times. The success of trophoblast isolation is confirmed by measuring the synthesis of human chorionic gonadotrophin in culture after three days by immunoassay (e.g., Hybritech). Trophoblast cells can be cultured as described in U.S. Pat. No. 5,503,981. The choriocarcinoma line, JEG-3, can be obtained from the American Type Culture Collection and cultured in RPMI-1640 medium supplemented with 10% fetal calf serum.

### 6.2. Example 2

### Preparation of Erythroblast Specific Antibodies

A large naive human phage-display library was constructed by recloning the heavy and light chain variable regions from the lox library vectors into the phagemid vector pHEN2 (Griffiths et al., Vaughan et al.). The library displays antibody as a single-chain variable region (scFv) molecule, comprising random combinations of germ-line V_{H} and V_{L} regions linked together as part of a single polypeptide chain.

Briefly, the kappa and lambda light chain variable regions were PCR amplified from the fdDOG-2lox Vκ and Vλ phage constructs using the following primers: 5′-GAG TCA TTC TCG ACT TGC GGC CGC ACG TTT GAT TTC CAS CTT GGT CCC-3′ (SEQ. ID NO:1) or 5′-GAG TCA TTC TCG ACT TGC GGC CGC ACC TAG GAC GGT CAG CTT GGT CCC-3′ (SEQ. ID NO:2) and “FdPCRback”: 5′-GCG ATG GTT GTT GTC ATT GTC GGC-3′ (SEQ. ID NO:3). The PCR fragments were purified and digested with ApaL1 and Not1. The gel purified fragments were then ligated into the vector pHEN2 in several aliquots. DNA was then purified from the ligation mixtures, resuspended in water, and electroporated into E. coli TG1. Vk-pHEN2 or VL-pHEN2 library pools of 3.5×107 and 1.67×107 respectively, were obtained. V_{H} regions were PCR amplified from the pUC19-2lox V_{H} vector using the primers “LMB3” 5′-CAG GAA ACA GCT ATG AC-3′ (SEQ. ID NO:4) and “CH1.LIBSEQ” 5′-GGT GCT CTT GGA GGA GGG TGC-3′ (SEQ. ID NO:5).

The PCR fragments were purified and digested with Sfi 1 or Nco 1 and Xho 1. The gel purified fragments were then ligated into the vectors Vκ-pHEN2 or Vλ-pHEN2. DNA was purified from the ligation mixtures, resuspended in water, and used for several hundred electroporations into E. coli TG1 to obtain a total library size of 2×10⁹. The scFv fragments contain a small c-myc peptide fused at the C-terminus as a tag to facilitate detection of the soluble scFv fragment using an anti-c-myc monoclonal antibody conjugated to horseradish peroxidase (HRP).

Human fetal erythroblast cells were prepared as described in Example 1, with or without papain treatment. The cell population from one liver was re-suspended in filtered PBS/2% marvel, which acted as a blocking agent against non-specific binding of phage to the cells.

Approximately 10¹³ phage from the phage display library (˜6×10⁹ human scFv clones) were incubated for 16 h with 3×10⁶ cells in filtered PBS/2% marvel to a final volume of 250 μL at 4° C. Cells were washed 5-6 times within 1 h, and then lysed in distilled water. The debris containing the phage was collected and used to infect an exponentially growing culture of E. coli TG1. Infected cells were grown overnight on plates containing 100 μg/mL of ampicillin and 2% glucose. The plates were scraped the next day, and phage were rescued from the selected population using M13 K07 as described in the art (Marks et al., 1991). Rescued phage were used to perform another selection, and the process was repeated until 3 rounds of selection had been carried out.

Individual colonies were grown in 96 well plates, and production of scFv was induced using 1 mM IPTG for 16 h. Clones were selected in an ELISA-format assay, using density-purified erythroblasts from fetal livers (positive selection), and adult red cells (negative selection). Cells were spun at 600 rpm for 5 min at 4° C. onto poly-L-lysine coated 96 well plates (NUNC, Immunosorb) 5×10⁴/well in 50 μL. Fixation was carried out by adding either 50 μL of 0.1% glutaraldehyde in PBS, or 2.5% paraformaldehyde in PBS to each well, and leaving for 15 min at room temperature (Forster et al.). Plates were washed 3 times in PBS and blocked for 1 h with PBS containing 2% skimmed milk protein (PBS-M) at 37° C. Culture supernatants were adjusted to 2% skimmed milk protein in PBS, and then incubated with the different cell populations. Bound scFv was detected with monoclonal mouse antibody 9E10 (Sigma), which recognizes the myc tag on the scFv, followed by alkaline phosphatase conjugated goat anti-mouse immunoglobulin (Sigma) (Griffiths et al.). An antibody recognizing carcinoembryonic antigen (CEA-6, Vaughan et al.) was used as a negative control. The majority of positive clones were specific for erythroblasts. PCR fingerprinting using restriction enzyme BstN1 was performed in the manner of Clackson et al. to identify clones with unique sequences.

Nucleic acid sequencing was performed by PCR amplification of the scFv insert using primers specific for flanking phagemid sequences. Inserts were amplified using primers 5′-CAG GAA ACA AGC TAT GAC-3′ (SEQ. ID NO:6), which sits upstream from the pelB leader sequence;, and “fdSeq1” 5′-GAA TTT TCT GTA TGA GG-3′ (SEQ. ID NO:7) which sits in the 5′ end of gene 3 (Marks et al. 1991). Sequencing templates were prepared using a Qiagen plasmid Midi Kit and sequenced using the ABI Prism Dye Terminator Cycle Sequencing Ready Reaction Kit with Amplitaq FS (ABI/Perkin Elmer).

Partial sequence has been obtained for Clones 22, 23, and 28. Complete sequence has been obtained for Clones 1 and 27. Surprisingly, Clone 1 comprised murine variable region sequences rather than human sequences. The nucleic acid sequence at the ends of the V_{H} and V_{L} region indicated that it had been constructed using murine-specific PCR primers. The clone had probably entered the human library or a subpopulation during replication or selection, either from contaminated glassware or from contaminated helper phage. The other selected clones all had human scFv sequences.

Clones expressing selected antibody were grown in 50-500 mL cultures, induced with 1 mM IPTG for 3-4 h, and a periplasmic extract was prepared. Immobilized metal affinity chromatography (IMAC) was used to purify the scFv using a hexahistidine tag at the carboxy terminus on NTA-Agarose (Qiagen).

### 6.3. Example 3

### Characterization of Erythroblast Specific Antibodies

Validation of binding specificity was performed by determining the ability of each antibody to identify or enrich erythroblasts from mixed cell populations.

The purified scFv was tested for its ability to label cord blood mononuclear cells. The cord blood cells were separated on Ficoll® as described in Example 1, and washed with PBE (PBS containing 0.5% BSA and 5 mM EDTA). The cells were incubated with the primary antibody at ˜25 μg/mL in 100 μL PBE for 1 h. After washing, the cells were incubated with a 1/50 dilution of phycoerythrin-conjugated anti-mouse antibody (Jackson Laboratories) in 100 μL PBE, incubated, and rewashed. Fluorescence was measured on a Coulter EPICS™ XL-MCL flow cytometer.

Exemplary clones were analyzed using flow cytometry. Some clones (e.g., Clones 17 and 18) have a positive shoulder beside the bulk of negative cells. Other clones (e.g., Clone 23) have two obvious peaks. Clones showing no staining in the direct labeling experiment (e.g., Clone 14) were judged as negative. Ficoll® purified adult and cord blood represent very heterogeneous cell populations. In this preparation, 15% of the cells were erythroid as judged by staining for glycophorin A and CD36. The labeling of rare cell populations or low-density antigen by the scFv could easily be obscured.

As an alternative to direct labeling, the antibodies were characterized by their ability to enrich for erythroblast cells by magnetic activated cell sorting (MACS). Ficoll® purified cord cells were labeled with scFv and the 9E10 secondary antibody, and enriched using paramagnetic beads coated with goat anti-mouse immunoglobulin. The studies were conducted using either cord blood mononuclear cells, or adult whole blood or buffy coat preparations doped with cultured cord blood cells. The cells were incubated at 4° C. in 200 ml of PBE containing 1/10 dilution of purified scFv. After 1 h, the cells were washed by spinning in 5 mL PBE at 390 g for 5 min. The cells were resuspended in 200 mL of PBE containing 500 ng of 9E10, incubated for 1 h at 4° C., and then washed. This was followed by incubating with microbeads coated with rat anti-mouse IgG1 (Miltenyi Biotec Ltd.) for 15 min at 4° C. The cells were washed once more, resuspended in 20 μL PBE, and loaded onto a pre-equilibrated MACS MS+/RS+ column clamped in a MiniMACS magnet. The column was washed with 2′1 mL of PBE, removed from the magnet, and the cells were eluted with 1 mL PBE pushed through with the supplied plunger. Eluted cells were either analyzed by flow cytometery, or were spun onto poly-L lysine coated slides and stained with benzidine/Wrights Giemsa stain.

In some experiments, enriched cells were analyzed using antibody specific for hemoglobin (Parsons et al.). Cell samples were spun onto poly-L-lysine coated slides (Shandon) at 800 rpm for 10 min in a Cytospin™ 3 (Shandon). Slides were left for at least 30 min to dry, and then fixed for 2 min in acetone:methanol:ethanol 3:1:1 at room temp (Thorpe et al.). Slides were washed for 5 min in PBS, and then blocked for 10 min with 10% goat serum in a humid box. They were then incubated with purified Hb-1 scFv diluted in PBS containing 10% goat serum for 1 h at room temp. The slides were washed in PBS and incubated with 9E10 antibody at 5 μg/mL for 1 h, rewashed, and incubated for 30 min with FITC-conjugated anti-mouse immunoglobulin (Sigma). After a final wash, the slides were mounted using Vectashield™ mounting medium (Vector Laboratories Inc.) and viewed using a fluorescence microscope (Olympus BX 40).

The various clones were analyzed using flow cytometry for their ability to enhance MACS enrichment. Clones 18 and 28 appear to bind erythroid cells covering a wider range of CD36 expression levels than the rest. Glycophorin A positive cells with lower levels of CD 36 expression are enriched, as shown by the high trailing edge of glycophorin-A staining cells with low CD36 expression. These are probably reticulocytes recovered on the Ficoll® gradient. Benzidine Wrights Giemsa staining showed these cells to be non-nucleated. It was concluded that the antigen is present on both immature and mature erythroblasts, and at least some reticulocytes (probably immature reticulocytes high in CD36, which are more common in cord blood), but not on mononuclear cells of adult blood. The antigen is believed to be distinct from CD36, since CD36 is expressed on monocytes which are not recognized by these clones.

Clones 17 and 20 give a lower signal by direct labeling of Ficoll® purified cord cells, but clearly enrich a population of erythroid cells. The trailing edge (high Glycophorin A and low CD36) present in the cells enriched using Clones 18 and 28 was not apparent for these clones, suggesting that antigen expression is turned off earlier. No cells were enriched from adult blood using Clone 17. It was concluded that these clones recognize an antigen present on all the erythroid stages recognized by CD36, but distinct from that recognized by Clones 18 and 28.

Clones 4, 11, and 23 enrich a similar population of erythroid cells from cord blood as Clones 17 and 20. However, the antigens recognized by these clones are different, since they also pull out a population of CD36 positive, Glycophorin A negative cells from adult and cord blood. This additional cell population had the size and granular morphology of monocytes.

Clones 9 and 14 are similar in V_{H} and V_{L} amino acid sequences, except for the CDR3 region of V_{H}. They also have a similar binding profile on adult and cord blood cells. By direct labeling, Clone 9 was barely above background and Clone 14 appeared to be negative. However, both enriched erythroid cells from cord blood. Two populations of Glycophorin A positive cells can be distinguished, particularly in the case of Clone 14. As well as the main CD36^{med} population observed in other clones, there is an additional CD36^{high} population obtained using Clone 14.

The majority of erythroid cells in cord blood are non-nucleated reticulocytes or red cells. In cord blood, there are approximately 137×10⁹ L⁻¹ reticulocytes and 0.89×10⁹ L⁻¹ erythroblasts. Even after Ficoll® purification, there is still a significantly higher proportion of non-nucleated erythroid cells compared with nucleated erythroid cells in cord blood. The binding profile of the antibody clones across the erythroid lineage was performed using cells from erythroid culture as a more even representation of cell types arising during erythropoiesis.

### 6.4. Example 4

### Characterization of Unique Erythroblast Antigens

The phagemid vectors containing the cloned scFv antibodies allow for expression either as pIII fusion protein on the surface of filamentous phage, or as soluble single-chain molecules. Staining of fetal erythroblasts was tested using both the whole phage (developed with anti-M13 antibody) and purified scFv (developed with 9E10 anti-myc). Whole phage of each of the first seven clones (Clones 1, 17, 18, 22, 23, 27, and 28) effectively stained fetal erythroblasts. The purified scFv from Clone1 showed consistent high levels of signal, but scFv from the other clones demonstrated irregular or less intense staining. This difference may be due to the amplification of the signal that occurs when using the phage but not the soluble scFv.

To increase stability and facilitate detection, the Clone 1 scFv was converted into an Fab antibody by fusing the sequences for V_{H} (SEQ. ID NO.8) and V_{L} (SEQ. ID NO.9) (a κ-chain variable region) to CH1 and Cκ. Fab clone VODOX1 was used as a backbone vector. The V_{H} and V_{L} in the vector were substituted with the V_{H} and V_{L} of Clone 1.

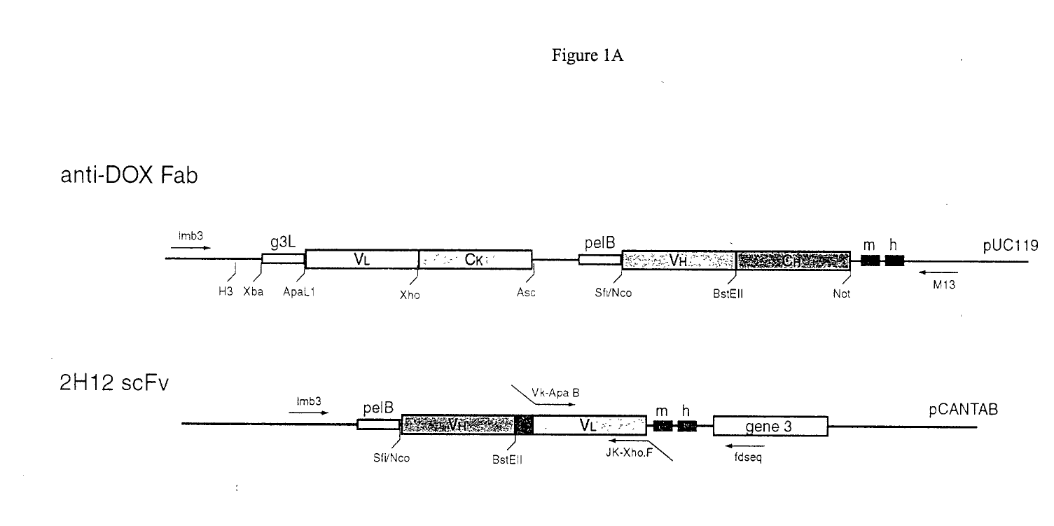

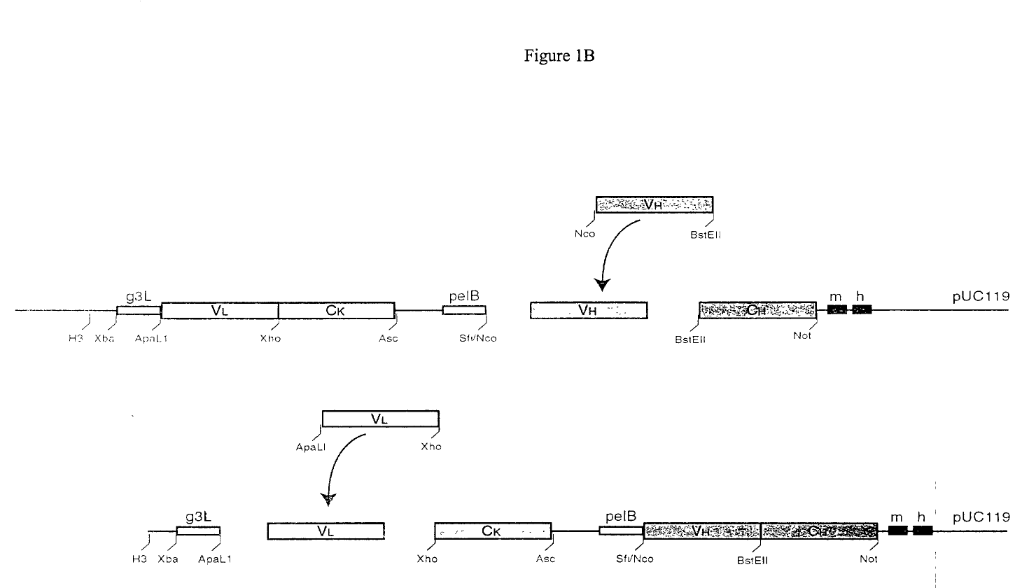

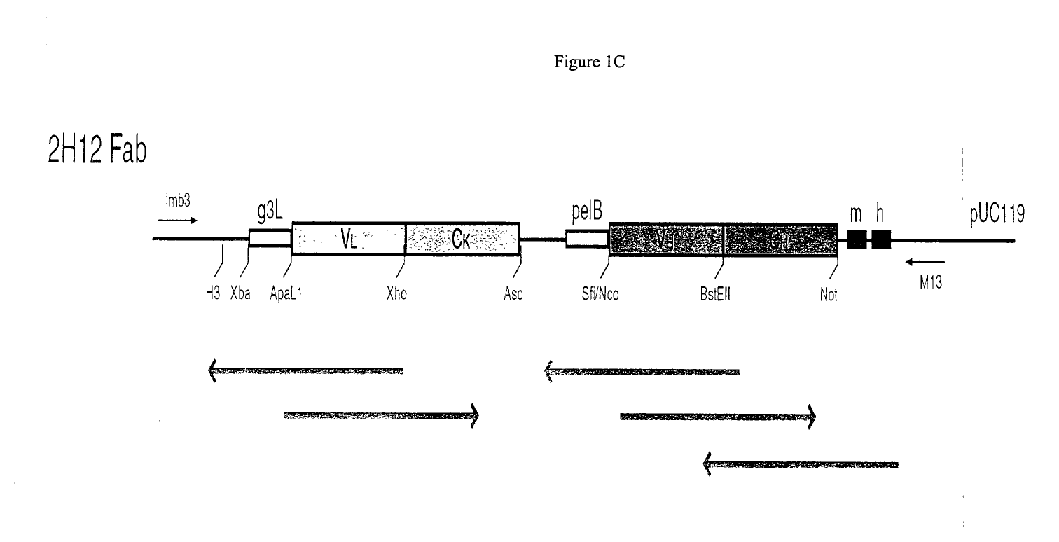

FIG. 1 shows the strategy for the substitution. An Nco1/Bste2 fragment containing V_{H} from clone1 was inserted into Nco1/Bste2 digested VODOX1. DNA was prepared from the cloning intermediate, and digested with ApaL1 and Xho1. Since these restriction sites are not present in Clone 1, the V_{L} was amplified as a PCR fragment with oligos containing these sites. The amplicon was digested and cloned as an ApaL1/XhoI insert. The resulting clone, designated Clone 1 Fab or e-Fab, was verified by sequencing through the regions indicated by the horizontal arrows.

Clone 1 Fab was expressed and successfully used to stain erythroblasts, with detection by either anti-myc or anti-human kappa. Clone 1 Fab was prepared using immobilized metal affinity chromatography (IMAC), in which the (His)6 tag of the antibody is captured on a nickel-loaded NTA column, purified by gel filtration, and then biotinylated. This reagent was used for both cell staining and cell separations.

To identify the antigen recognized by the erythroblast antibodies, each antibody was used for affinity isolation from cell extracts. Antibody was rescued from the extract along with bound antigen by IMAC. Both the scFv and the Fab constructs contain the (His)₆ tag, so either can be used. Fab was generally chosen when it was available, in part because it is expressed at much higher levels than the scFv.

Erythroblast cells were surface labeled with biotin and lysed using a Cellular Labeling and Immunoprecipitation Kit (Boehringer Mannheim) according to the manufacturer's instructions. Lysates from 10⁷ cells were incubated with Clone 1, either in the form of scFv or Fab, for 2 hrs. at 4° C. Antibody-antigen complexes were then recovered with nickel-NTA resin. The resin was then eluted with 250 mM imidizole, and the eluted protein was analysed by polyacrylamide gel electrophoresis in sodium dodecyl sulfate (SDS-PAGE). Gels were stained directly, or electroblotted to PVDF membrane for Western analysis. Since proteins from the cell surface had been biotinylated, they were detected using a conjugate of streptavidin-horseradish peroxidase (HRP), followed by a chemiluminescent substrate for HRP. The advantage of this system is that antigen molecular weight information can be obtained from crude cellular extracts and crude antibody preparations.

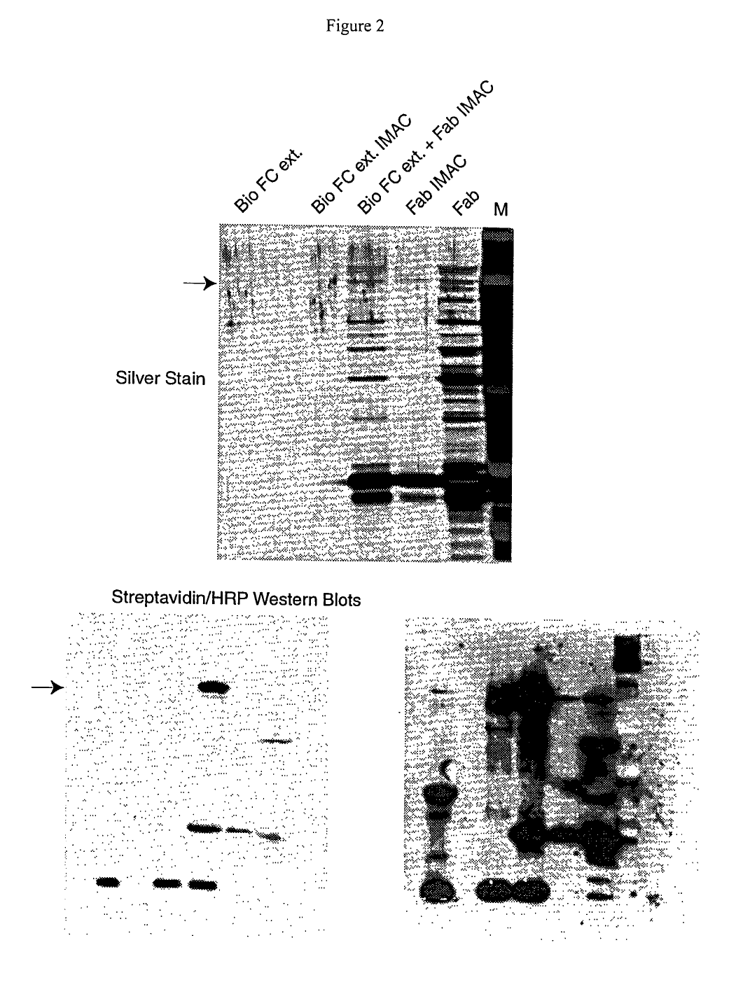

FIG. 2 shows the results of IMAC immunopurification using Clone 1 Fab. The top panel shows the silver stained gel (total protein); the two lower panels show the chemiluminescent patterns from the Western blots at two different exposures. A band corresponding to an apparent size of 90 kDa (arrow) was seen in the blot in the lane corresponding to the Fab absorbed biotinylated fetal cell extraction (Bio FC ext.), but not in the extract-only or Fab-only control lanes. A number of minor bands appear in the other lanes after a long exposure, but not with the same intensity or molecular weight as the antigen band. The 90 kDa antigen detected on the Western blot did not correspond to any of the prominent bands on the silver stained gel. Most of the protein represented by the silver stain corresponds to material present in the Fab antibody preparation.

In an alternative purification strategy, unlabeled extract was prepared from 5×10⁸ erythroblasts (a 50-fold increase from the previous purification). The extract was combined with biotinylated Fab under conditions that permitted binding to the solubilized erythroblast antigen. The antigen-antibody complexes were then captured using streptavidin-coated Dynabeads™. Antigen was eluted and analyzed.

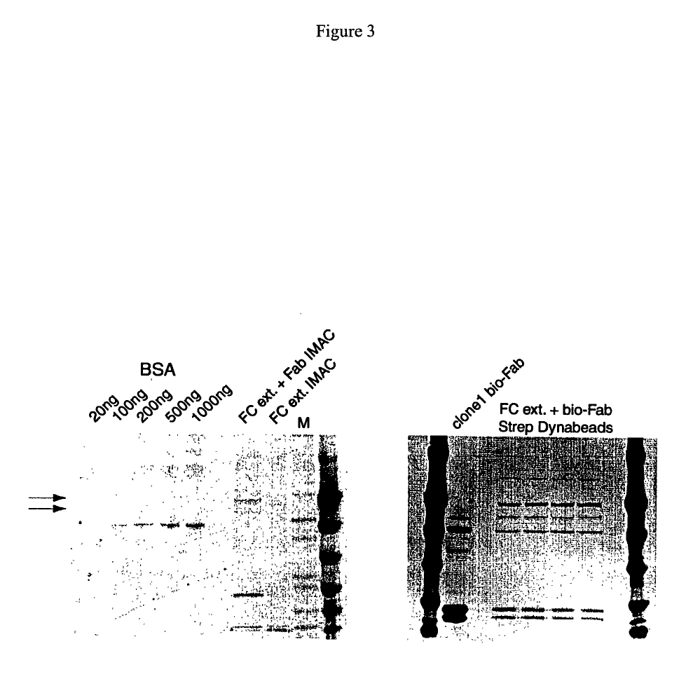

FIG. 3 shows quantitation and molecular weight analysis of antigen obtained from preparative-scale isolations by Ni-NTA purification (upper panel) or Dynabead purification (lower panel). Apparent molecular weights were calculated from the relative mobility on a semi-log plot using six molecular weight standards between about 150 and 30 kDa. In addition to the ˜90 kDa band seen previously, another specific but less intense band was seen at ˜78 kDa. It is not known whether the two bands represent separate cross-reacting antigens, or whether the 78 kDa species is an alternative form of the 90 kDa species.

The Coomassie stained transfer blot shown in the lower panel was used to obtain purified material for amino acid sequencing. The band at 90 kDa was cut out from each of the four bands, and pooled, yielding approximately 3 μg of purified material. No sequence was obtainable, and apparently the amino terminus of the protein is blocked.

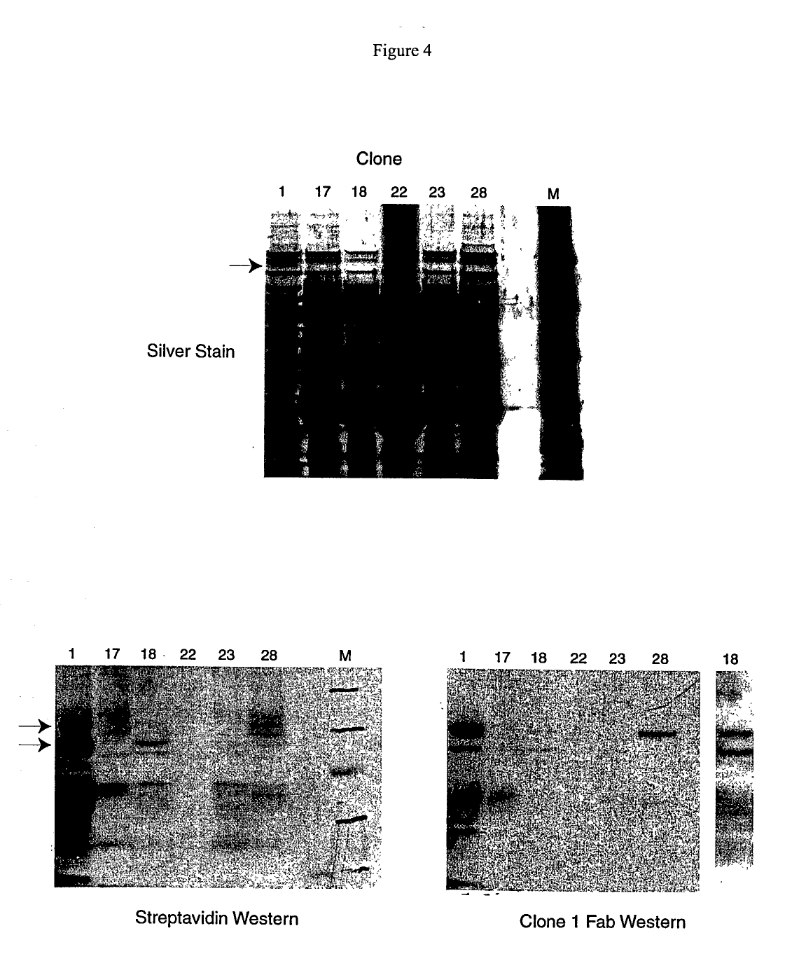

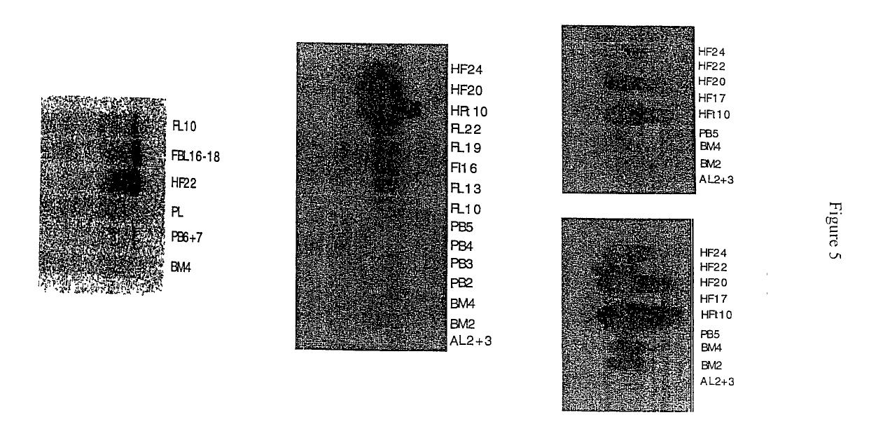

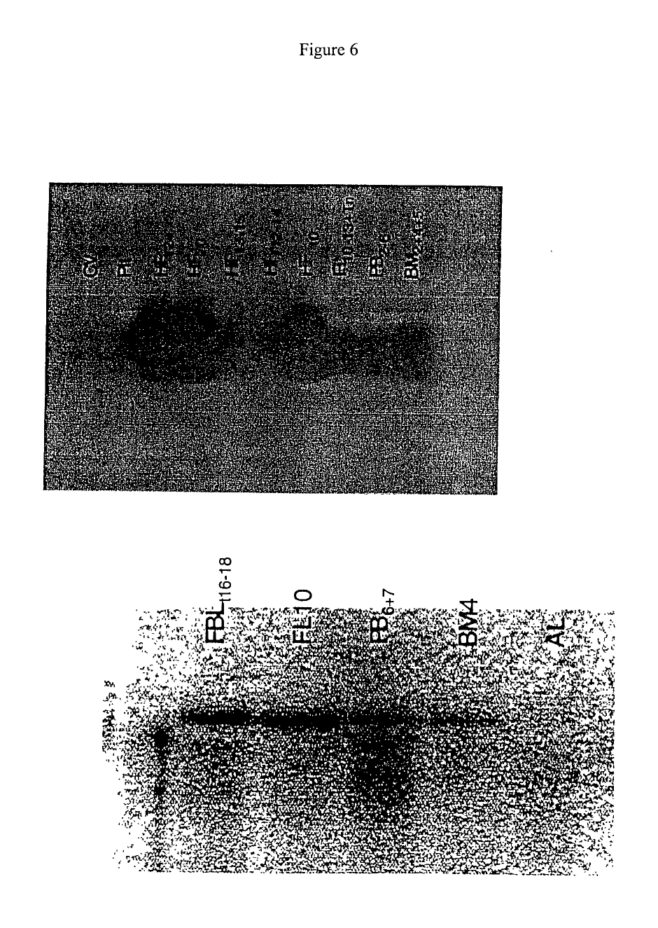

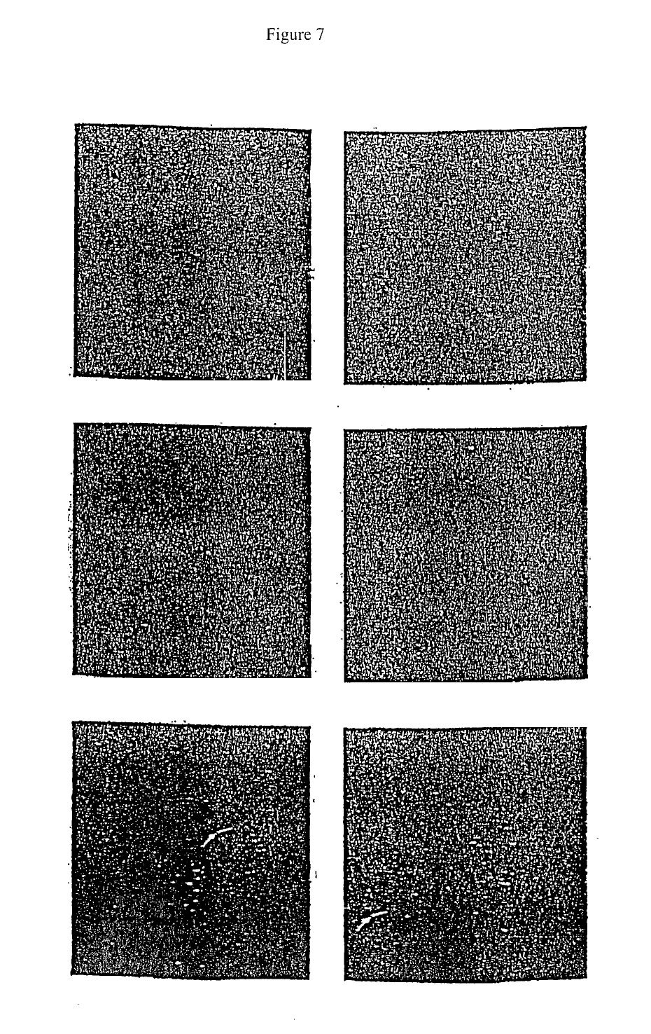

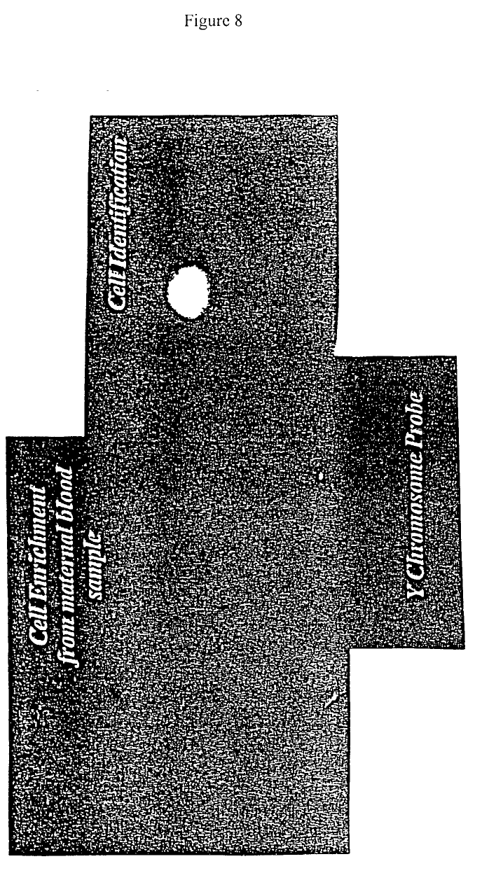

FIG. 4 shows the results of using the other cloned antibodies in the first group to purify biotinylated erythroblast membranes. Upper Panel: Silver stain; Lower Panel: Western blot from two separate experiments. The arrows indicate the position of the 90 kDa and 78 kDa bands identified by Clone 1. Clone 18 and Clone 28 appear to recognize the same bands, although the 90 kDa and 78 kDa species appear to be recognized in different proportions by the different clones. It is not clear from this experiment what antigen is recognized by Clone 17, 22, or 23. The antigens may be less abundant, or they may label with biotin less efficiently. Further characterization is performed using gel-purified scFv or Fab in a scaled-up procedure.

A summary of the Clones with established anti-erythroblast activity and their known antigen characteristics is shown in Table 2.

TABLE 2

| Designation | Cell Specificity | Antigen Characteristics |
|---|---|---|
| Clone 1 & 27 | | 90 kDa, 78 kDa |
| Clone 4 | erythroblasts & monocytes | |
| Clone 9 | early erythroblasts | |
| Clone 11 | erythroblasts & monocytes | |
| Clone 13 | | |
| Clone 14 | early erytbroblasts | |
| Clone 17 | early erythroblasts | |
| Clone 18 | erythroblasts & early | 78 kDa, (90 kDa) |
| | reticulocytes | |
| Clone 20 | early erythroblasts | |
| Clone 22 | | |
| Clone 23 | erythroblasts & monocytes | |
| Clone 28 | erythroblasts & early | 90 kDa, 78 kDa |
| | reticulocytes | |

### 6.5. Example 5

### Erythrocyte-Specific Antibodies Obtained by Both Positive and Negative Selection

Additional erythrocyte-specific antibodies were obtained using a modified scFv library. A naïve library of Fab expressing phagemids (about 10¹⁰ species) was converted so as to express the variable regions as scFv. The heavy chain CDR3 regions were scrambled to provide additional diversity.

Specific anti-erythroblast antibodies were obtained by a combination of positive and negative selection. Erythroblasts from fetal liver that had been cultured for 1-2 weeks were used for positive selection. Adult peripheral blood leukocytes (PBL) (pooled Ficol™ separated white cells) were used for negative selection. Briefly, the phagemid library was mixed with the erythroblasts. The bound phagemids were then recovered from the cells by adding 0.1 M glycine buffer pH 2.2, inclubating for 5 min, and centrifuging out the cells. The supernatant was neutralized by adding concentrated Tris buffer. The recovered particles were then replicated. Positive selection using the erythroblasts was repeated twice for a total of three rounds. The selected phage were then negatively selected by incubating with peripheral blood leukocytes, the supernatant was recovered. The phagemids in the supernatant were then positively selected with erythroblasts, and replicated as before. The recovered phagemids were then subjected to another round of negative and positive selection.

An aliquot of phagemids was saved from each of the selection steps for subsequent analysis. Specificity was determined by conjugating with biotin, incubating with erythroblasts, and developing with streptavidin coupled with Texas Red™.

The results of this analysis demonstrated weak staining using the phage mixture obtained after three rounds of positive selection. Much stronger selection was obtained after one or two subsequent rounds of PBL subtraction followed by erythroblast enrichment.

### 7. EXAMPLE

### Identification of Fetal Cell Associated Transcripts

### 7.1. Example 1

### Construction of Subtracted cDNA Libraries

This example is directed at identifying nucleic acid sequences that are expressed at the mRNA level in fetal cells appearing in the maternal circulation, but not in any type of circulating maternal cells that might be present in a test sample after antibody enrichment. Where the target fetal cell is a blood cell precursor such as an erythroblast, the sequence should be able to distinguish fetal cells from maternal cells at the same stage of differentiation.

To accomplish this, a number of cDNA subtraction libraries were prepared in which sequences specifically expressed in fetal cell precursors are enriched. The libraries were prepared by Suppression Subtraction Hybridization (SSH), a PCR-based method that combines normalization (the matching of mRNA levels) and subtraction (obtaining differentially expressed mRNA) in a single procedure, and requires less mRNA than other subtraction methods.

Different tissues were obtained as both the source of the differentially expressed mRNA (referred to as the “tester”) and the source of baseline mRNA that would be subtracted (referred to as the “driver”).

RNA Isolation: Total RNA was isolated using the TRIZOL™ Reagent (Cat #15596-026) from Life Technologies (Gaithersburg, Md.) according to manufacturer's instructions. For mRNA isolation either the Straight A's™ mRNA Isolation system (Cat #69962-1) from Novagen (Madison, Wis.) or the mRNA Purification System (Cat #27-9258-02) from Pharmacia (Piscataway, N.J.) was used according to manufacturer's instructions. The RNA preparations were used immediately or stored at −70° C.

cDNA synthesis: The RNA was reverse transcribed using either conventional methods as described by Klickstein, L. B., Neve, R. L., Golemis, E. A., and Gyuris, J., 1995, in Current Protocols in Molecular Biology, Ausubel, F. M., et. al., Eds, John Wiley & Sons, Inc., New York, N.Y., pp. 5.5.1-5.5.10 for at least 2 μg mRNA or CapFinder™ kit (Cat #K1052-1) from Clontech Laboratories, Inc. Palo Alto, Calif. for less than 1 μg total RNA. The CapFinder™ synthesis was performed essentially as described in the product insert; the only change was that the PCR amplification conditions were conducted with 27 cycles of 95° C. for 12 seconds and 68° C. for 4 minutes.

SSH: Subtraction suppression hybridization (SSH) was conducted using a PCR-Select™ kit (Cat #K1084-1) from Clontech Laboratories, Inc. Palo Alto, Calif. according to manufacturers instructions except for the following modifications. The first and second hybridizations were performed for 14 and 22 hours, respectively. After adaptor extension, the PCR amplification conditions were 28 cycles of 95° C. for 15 seconds, 65° C. for 25 seconds and 72° C. for 2 minutes. Then, the PCR product was re-amplified for 15 cycles at 94° C. for 10 seconds, 68° C. for 25 seconds and 72° C. for 2 minutes.

The following table shows the different combinations of tester cDNA and driver cDNA that have been used for preparing suitable subtraction libraries:

TABLE 3
Summary of Subtracted cDNA Libraries

| Subtracted cDNA | | | | | | | |
|---|---|---|---|---|---|---|---|
| Library Series | Tester | | | Driver | | | Bioblock |
| FL10/22 | 10 w FL | mRNA | CM | 22 w FL | mRNA | CM | N/A |
| FBP10-12/BM | 10-12 w FBP | mRNA | CF | | mRNA | CF | N/A |
| HFt10-11/BM | 10-11 w HF | total | CF | BM | total RNA | CF | 20-99 |
| | | RNA | | | | | |
| HF13/BMPB | 13 w HF | mRNA | CF | BM & PB (1:1) | mRNA | CF | 100- |
| HFt 12-14/24 | 12-14 w HF | total | CF | 24 w HF | total RNA | CF | 200- |
| | | RNA | | | | | |
| HFt 12-14/BMPB | 12-14 w HF | total | CF | BM & PB (1:1) | total RNA | CF | 400- |
| | | RNA | | | | | |
| FBLt12-14/BMPB | 12-14 w FBL | total | CF | BM & PB (1:1) | total RNA | CF | 300- |
| | | RNA | | | | | |
| FBLt12-14/24BMPB | 12-14 w FBL | total | CF | 24 w FBL & | total RNA | CF | 500- |
| | | RNA | | BM & PB | | | |
| | | | | (1:1:1) | | | |
| FBt12-14/22-24 | 12-14 w FB | total | CF | 22-24 wFB | total RNA | CF | 1000- |
| | | RNA | | | | | |
| FBt12-14/BMG | 12-14 w FB | total | CF | BM & | total RNA | CF | 1500- |
| | | RNA | | g-GLOBIN | | | |
| | | | | (5:1) | | | |

Abbreviations:
FL: Human fetal liver
CM: cDNA from conventional methods
FBP: Porcine fetal blood
CF: cDNA from CapFinder ™ synthesis
HF: Human fetal cord or circulating blood
BM: Human adult bone marrow
PB: Human adult peripheral blood
FBL: Human fetal blood from liver

The amplified cDNA was digested with Rsa I to remove the adaptor sequences, size selected on a 2% agarose gel, and subcloned into the PCR-Script™ (SK+) vector (Cat #211189, Stratagene, La Jolla, Calif.). This represents a selected cDNA library by SSH.

### 7.2. Example 2

### Identification and Characterization of Short Fragment cDNAS (Tags) from Subtracted cDNA Libraries

Random clones from the subtracted libraries were picked and grown to provide sufficient material for characterization. The cDNA insert was PCR amplified for further testing using primers (T3 and T7) corresponding to the flanking sequences of the pCR-Script™ (SK+) vector (Cat #211189, Stratagene, La Jolla, Calif.) as designated in the package insert. The PCR products were purified according to manufacturers instructions using the PCR purification kit (Cat#28106) from Qiagen (Valencia, Calif.). The tags were single pass sequenced from one end using Dye-Terminator chemistry on a 377 ABI fluorescent DNA sequencer (PE Biosystems, Inc. Foster City, Calif.). Using either the BLAST (Altschul, S. F., Gish, W., Miller, W., Myers, E. W. & Lipman, D. J. (1990) “Basic local alignment search tool.” J. Mol. Biol. 215:403-410.) or the BLAST2 algorithm (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs,” Nucleic Acids Res. 25:3389-3402), the tag sequences were compared to the Genbank public database (NCBI, The National Center for Biotechnology Information) to see if the tag represented a fragment of a known gene. If not, the tag was subjected to further analysis. These clones were further analyzed first by Southern blotting of amplified cDNA fragments from a panel of 6-8 tissues representing mostly adult and fetal blood and blood forming organs. Briefly, 0.5 μg of amplified cDNA from relevant tissues were electrophoresed on a 1.2% agarose gel, transferred to a nylon filter and hybridized with a ³²P randomly labeled (Tabor, S., Struhl, K., Scharf, S. J., and Gelfand, D. H., 1997, in Current Protocols in Molecular Biology, Ausubel, F. M., et. al., Eds, John Wiley & Sons, Inc., New York, N.Y., pp. 3.5.9-3.5.10) candidate tag. The hybridization conditions used were as shown by George M. Church and Walter Gilbert, 1984, Genome Sequencing, Proc. Natl. Acad. Sci. 81:1991-1995.

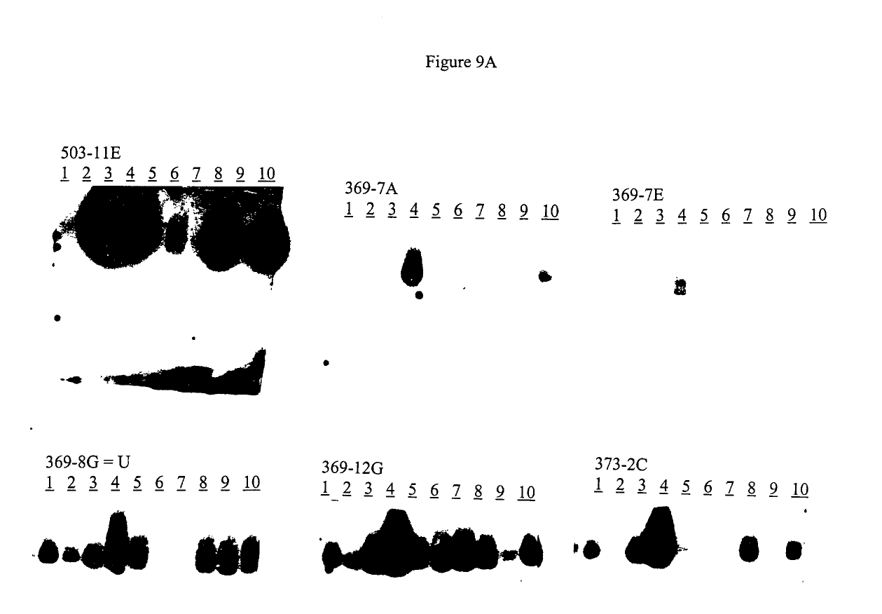

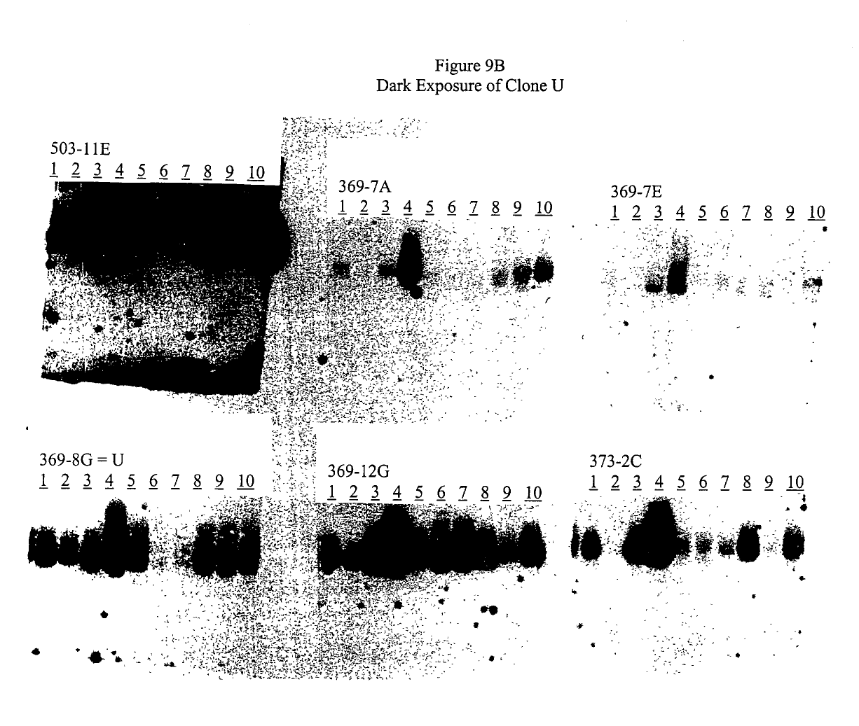

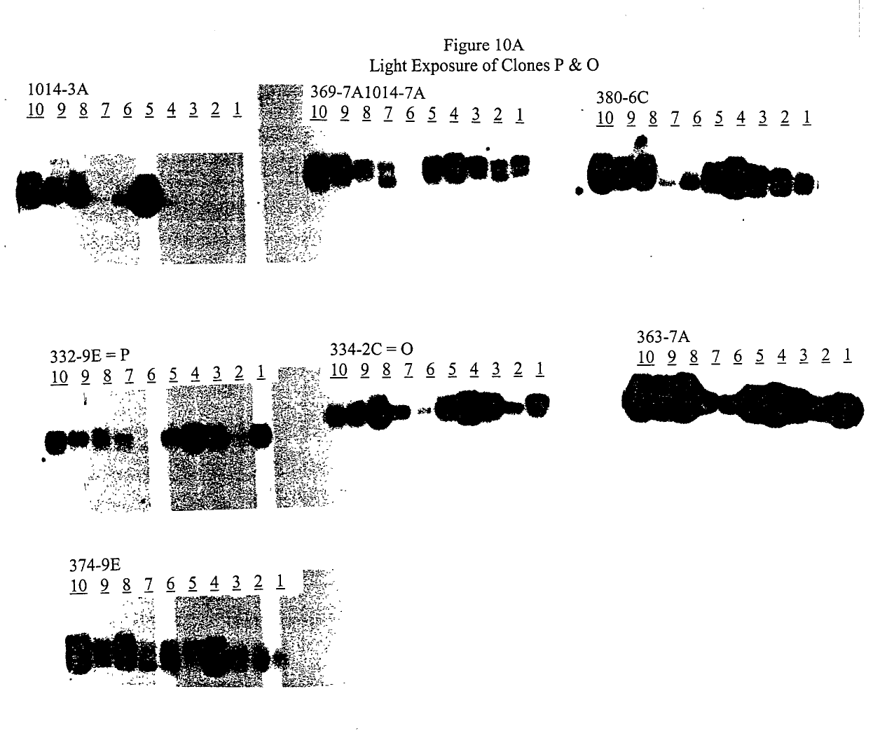

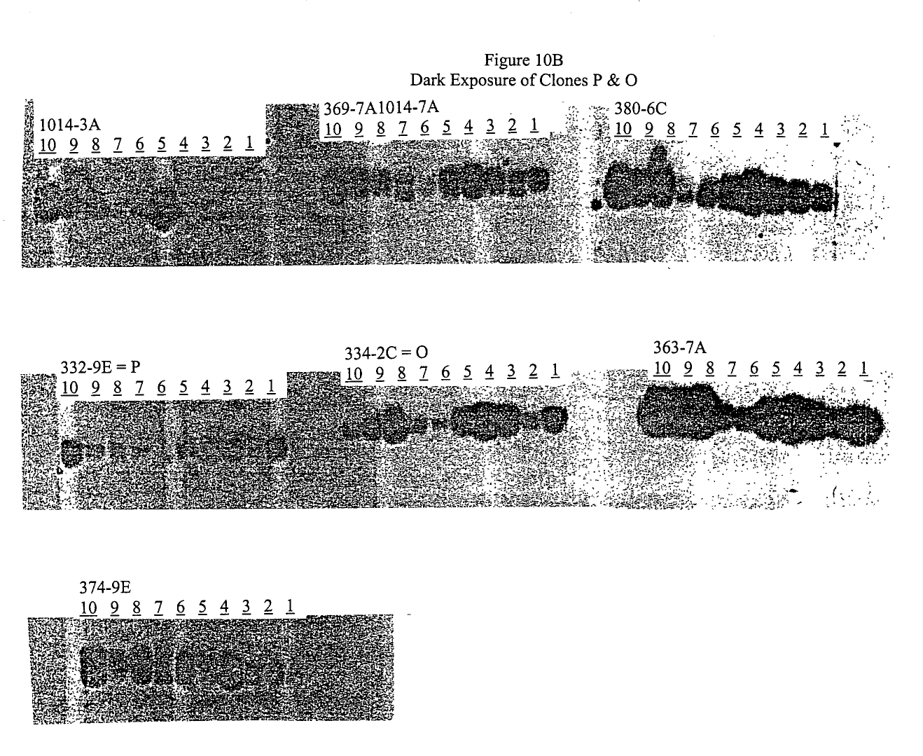

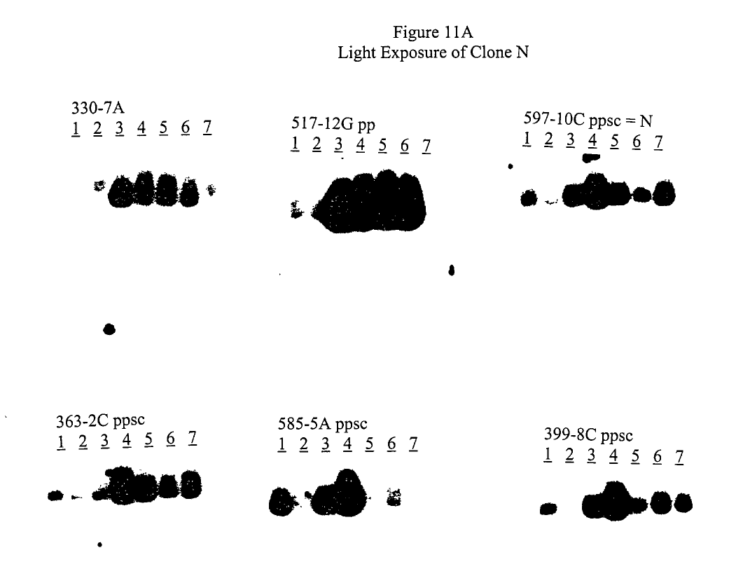

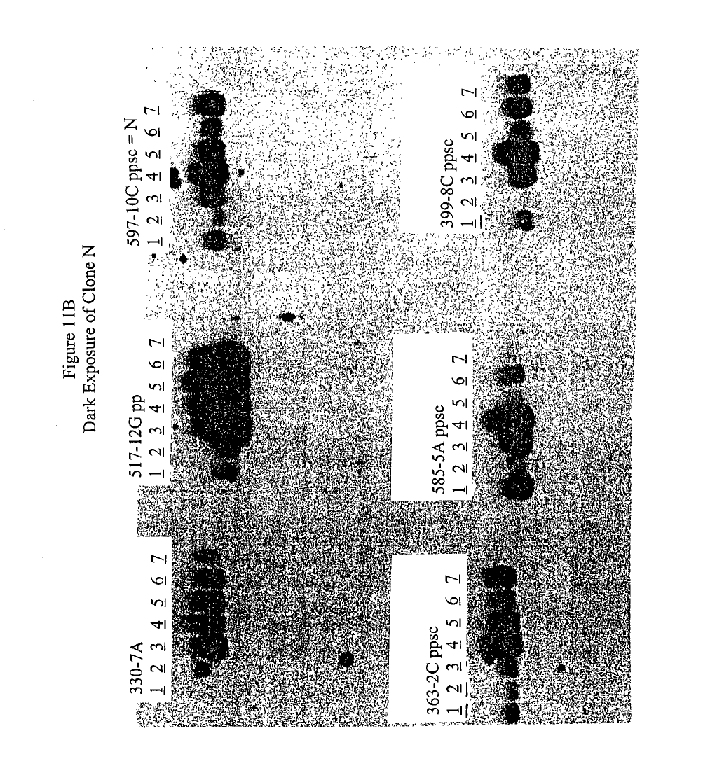

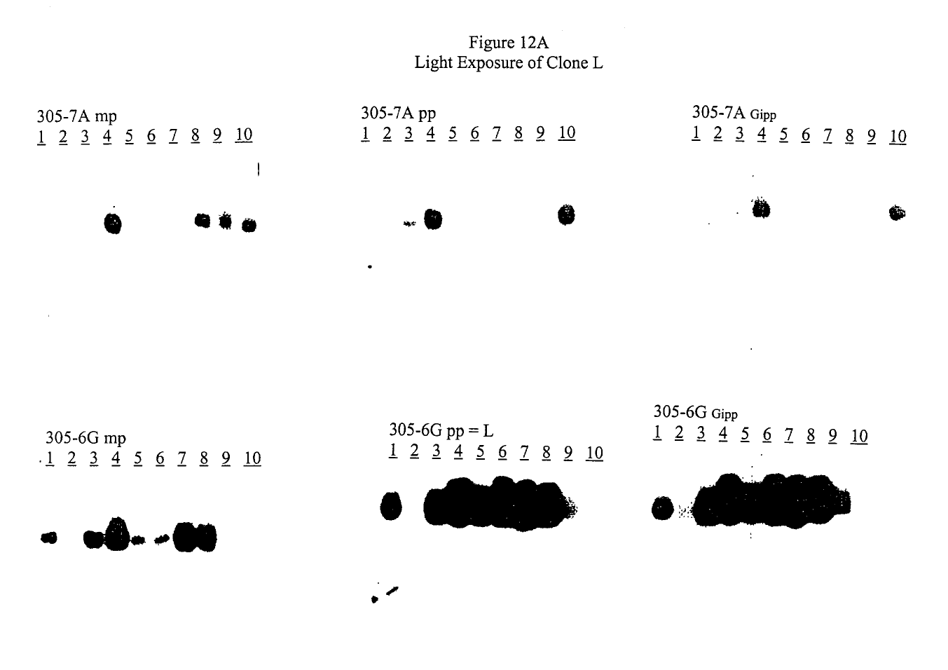

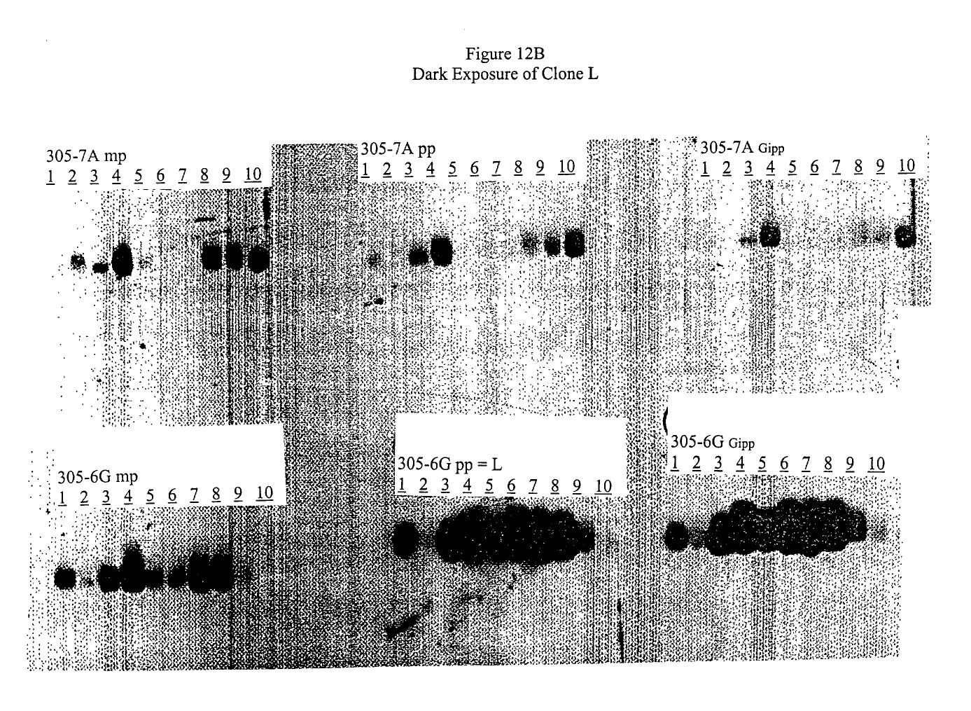

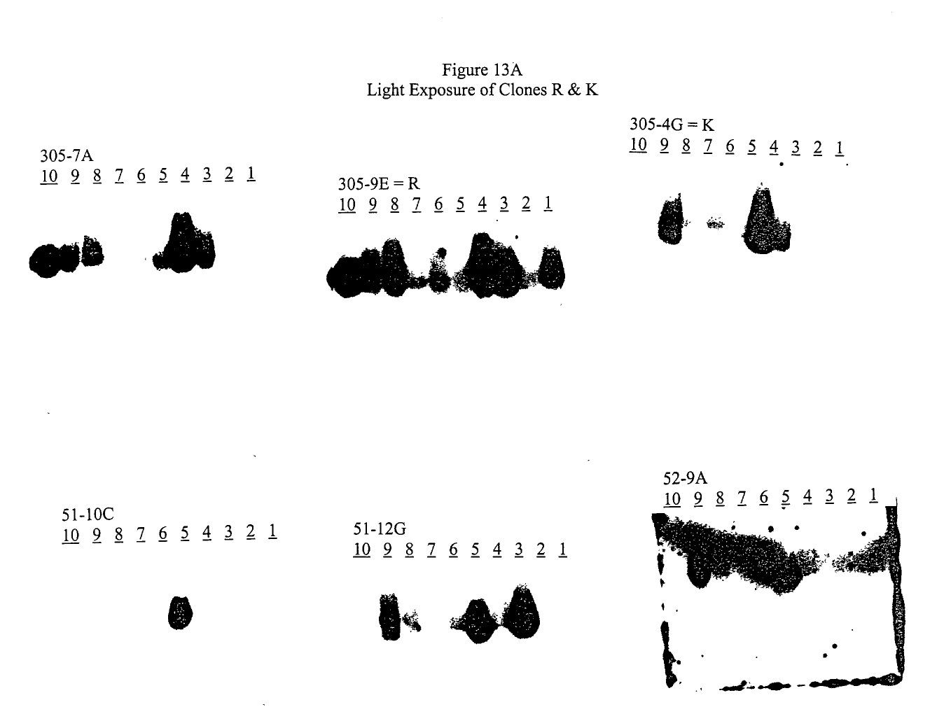

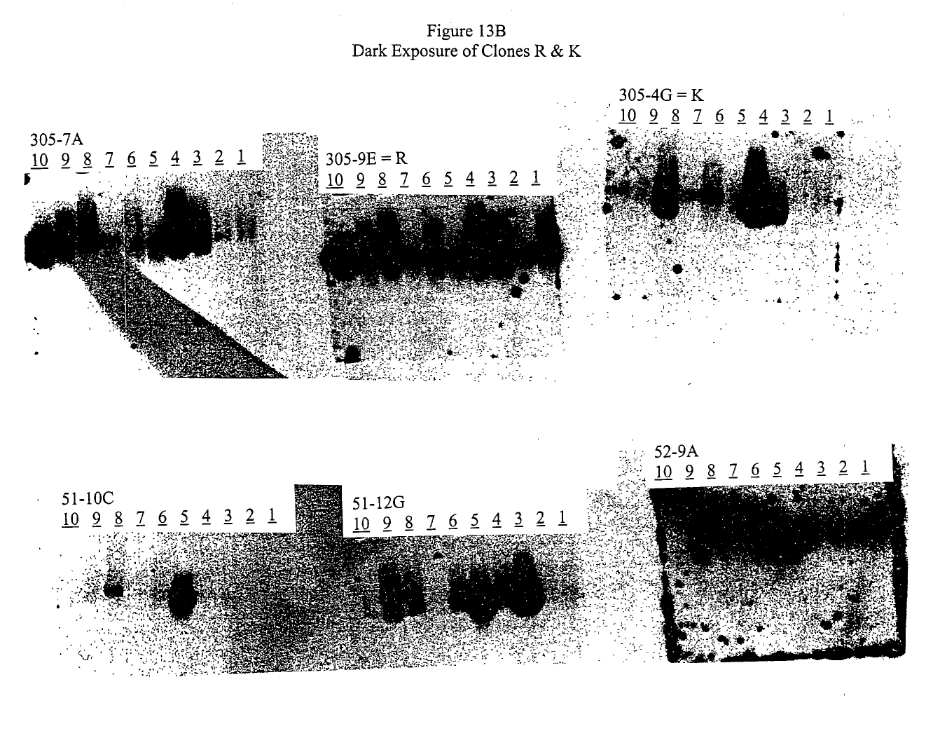

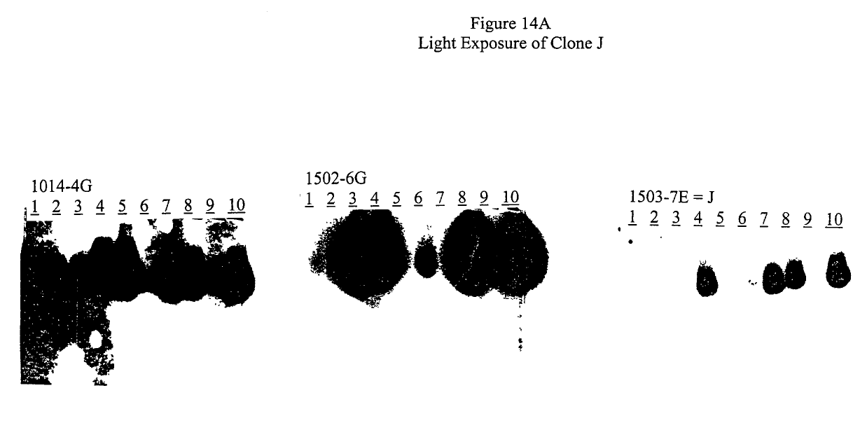

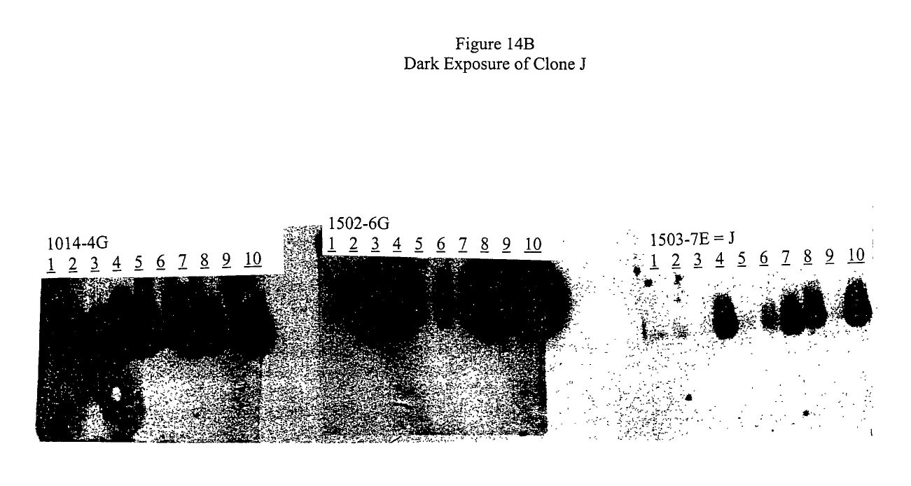

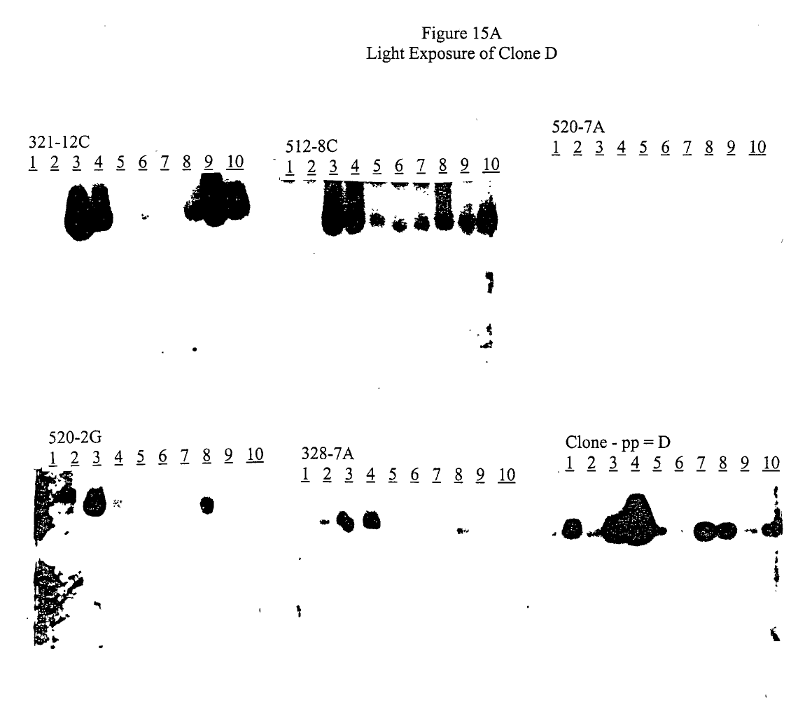

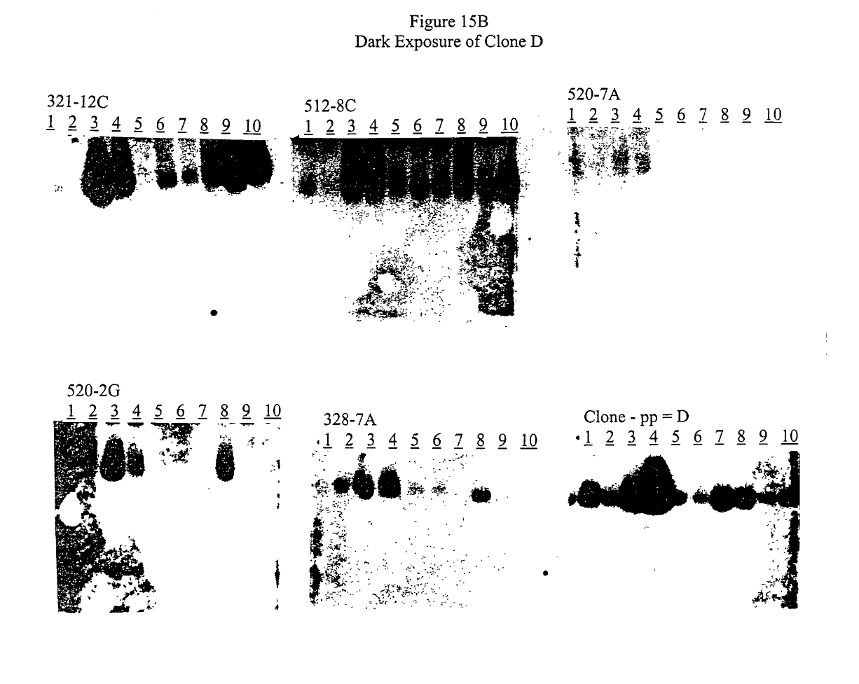

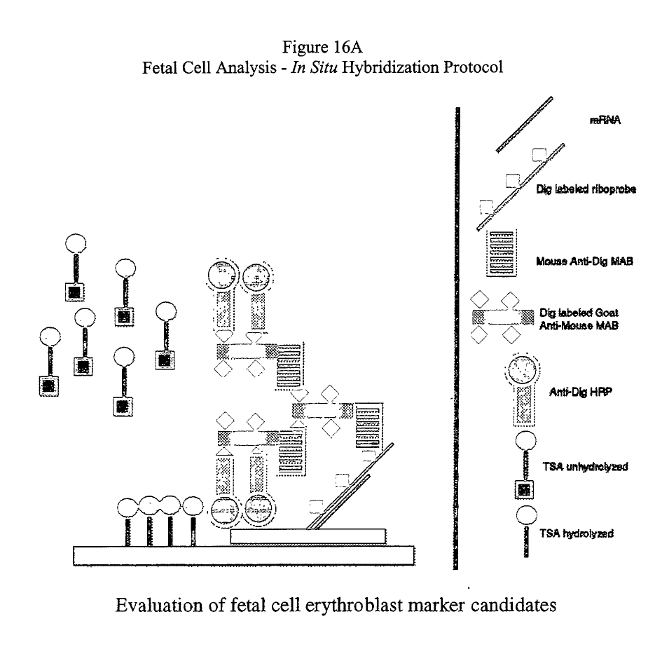

Tags showing evidence of specificity for fetal cells compared to adult cells were further analyzed by Southern blotting with an extended cDNA panel representing tissues from more individuals, tissue types and gestational time points. This analysis was then expanded to testing the differential tags directly on total RNA and mRNA isolated from adult and fetal blood and blood forming organs on a Northern blot. For preparation of the Northern blot, 20 μg of total RNA or 2 μg of mRNA isolated from relevant tissues was loaded onto a denaturing agarose gel. After electrophoresis the separated RNA was then transferred to a nylon membrane and hybridized with the tag under analysis. Tags were labeled with ³²P either by random priming (Tabor, S., Struhl, K., Scharf, S. J., and Gelfand, D. H., 1997, in Current Protocols in Molecular Biology, Ausubel, F. M., et. al., Eds, John Wiley & Sons, Inc., New York, N.Y., pp. 3.5.9-3.5.10) or by asymmetric PCR (Peter C. McCabe, Production of Single Stranded DNA by Asymmetric PCR, in PCR Protocols, A Guide to Methods and Applications Michael A. Innis et. al., Eds. 1990 Academic Press, pp 76-83. The hybridization conditions used were as described by Brown T., and Mackey, K., 1997, Current Protocols in Molecular Biology, Ausubel, F. M., et. al., Eds, John Wiley & Sons, Inc., New York, N.Y., pp. 4.9.1-4.9.8. For each candidate tag, direct RNA analysis (by Northern blot) was performed to assess the following: validate the expression patterns seen in the cDNA blots, determine the number of mRNAs that hybridized with each promising tag, assess the size of the mRNA and to determine which strand of the tag represented the coding strand of the messenger RNA. Results of blotting experiments are shown in FIGS. 9-15.

Tags that passed to this point were then evaluated by mRNA Fluorescence in situ hybridization for their expression in the cytoplasm of individual fetal and adult erythroblast cells, and all adult end-stage nucleated peripheral blood cells.

To begin with, riboprobes representing each tag to be evaluated were synthesized and titered on fetal liver blood cells and adult peripheral blood. After validation of each of the probes' reactivity in the cytoplasm of fetal liver erythroblasts and not in adult nucleated peripheral blood cells they were tested on many other relevant tissues representing numerous individuals. These included circulating fetal erythroblasts (fetal cord blood pools from 8-12 week gestation human fetuses), adult erythroblasts (adult human bone marrow from iliac crest enriched for erythroblasts using an anti-transferrin receptor antibody) and adult human nucleated peripheral blood cells (white cell fraction from whole adult blood). To allow more adult erythroblasts for analysis, adult bone marrow mononuclear cells were erythroid enriched using an anti-transferrin receptor antibody attached to solid phase; this resulted in bone marrow preparations that were 90% erythroid compared to 20-35% without enrichment.

Experiments were set up to test all these populations with each probe beginning with hybridization conditions of lower stringency and moving to higher stringency. This was done by varying experimental parameters such as: increasing the temperature of hybridization (from 55° C. to 60° C.), increasing the temperature of the washes (from 55° C. to 60° C.), decreasing the salt concentration in the washes (0.2×SSPE to 0.05X SSPE), varying the number of washes of each type (2-3), and finally, the addition of 100 mM TMAC to the hybridization, wash and moist chamber buffers. Through all these experimental alterations cellular morphology was preserved and by accessing the hybridization signal in multiple tissues across many stringency conditions the likelihood of a spurious positive was minimized. Hybridization signal should rise and fall according to stringency condition in a predictable manner if it is based on specific binding interactions of a nucleic acid probe with its target.

Single cell preparations were made; all according to standard cell biology methods (Cell Biology, A Laboratory Handbook 2nd Edition, J. E. Celis, Ed., Academic Press, 1998), from pooled and washed 8-12 week gestation fetal human cord blood (high in nucleated red blood cells), adult bone marrow mononuclear cells (Cat #1M-125A, Biowhittaker, Gaithersburg, Md.) enriched for the nucleated red cell precursors and progenitors with an anti-transferrin receptor antibody attached to a solid phase (A. A. Neurauter, et. al., Immunomagnetic Separation of Animal Cells, pp 197-204, in Cell Biology, 2nd Edition, J. E. Celis, Ed., Academic Press, 1998), adult peripheral blood mononuclear cells either by density gradient fractionation on Histopaque 1077 (Sigma Chemical, St. Louis, Mo.) according to manufacturer's instructions; or by lysis of red blood cell fraction, as described by McCoy Jr., J. P., 1998, in Current Protocols in Cytometry, Robinson J. P., et. al., Eds, John Wiley & Sons, Inc., New York, N.Y., pp. 5.1.2-5.1.3, and developing blood cells from first trimester human fetal liver. Briefly, blood cells were released from first trimester human fetal liver by floating the liver in a small petri dish in minimal essential alpha medium (Gibco BRL/Life Technologies, Grand Island, N.Y.; Cat#32561-037) and gently scoring the surface with a scalpel and swirling the dish to release the cells. Medium containing the fetal blood cells in the filtered through a 74 μm mesh screen (Costar/Coming, Corning, N.Y.; Cat#3479) into a 50 ml centrifuge tube and cells were pelleted by centrifugation in a Megafuge® 1.0R/2.0R (Kendro Laboratory Products, Newtown, Conn.) at 300×g for 10 minutes at 4° C. Cells were counted and attached to coated glass microscope slides (Shandon®)) using the Shandon® Cytospin 3 system and centrifugation conditions of 600 rpm, medium acceleration for 3 minutes (Shandon Lipshaw, Inc., Pittsburg, Pa.). Slides were handled with gloves and always in an RNAase free manner. Slides were fixed with 4% Paraformaldehyde/5% Acetic acid for 20 minutes, washed 2 times in PBS and once in Molecular Grade water (Genotech Cat #78672), dried on a 37° C. slide warmer and either used immediately or stored at −20° C. in airtight containers containing dessicant. The in situ hybridization method used was based on Rosen B. and Beddington R., Detection of mRNA in whole mounts of mouse embryos using digoxigenin riboprobes (1994) in Methods in Molecular Biology Vol 28, Issac P. G., Ed. Humana Press Inc., Totowa, N.J. Since we were using single cells some modifications were made. Slides containing fixed cells were permeabilized with Proteinase K (0.1 μg/ml PBS) for 10 minutes at room temperature. Cells were then postfixed with 4% paraformaldehyde/5% acetic acid for 5 minutes, rinsed in Molecular grade water twice for 5 minutes each, incubated in 0.05% Saponin/PBS twice for 10 minutes each, and rinsed in PBS twice for 1 minute each. Hydrogen peroxidase activity was blocked using 3% Hydrogen peroxide/1% Sodium azide/PBS for 40 minutes. Slides are rinsed in Molecular grade water twice for 1 minute, subjected to an ethanol gradient (70%, 95%, and 95% in Molecular grade water for 1 minute each) and allowed to air dry for 10 minutes.

For each tag, digoxigenin labeled riboprobes that were complementary to the sense mRNA strand were synthesized using a method based on (Signer, S. N., Digoxigenin Labeling of RNA Transcripts from Multi- and Single-Locus DNA Minisatellite Probes pp. 77-81, in Methods in Molecular Biology Vol. 28, Ed: Issac, P. G., 1994 Humana Press, Inc., Totowa, N.J.). The following modifications were made: Molecular Grade water was used throughout, the final reaction volume was increased to 23 μl, and the template was destroyed with RQ1 DNase (Promega, Madison, Wis., Cat #M610A). To control the final probe size to approximately 150-200 base pairs the precipitated riboprobes were subjected to controlled alkali hydrolysis at 65° C. (Anderson, M. L. M., 1999, Nucleic Acid Hybridization, Springer-Verlag New York Inc., pp. 125). Hydrolyzed probes were then re-precipitated and 10% of each was analyzed on a denaturing agarose gel to determine the extent of the hydrolysis.

Slides were hybridized with denatured probe (mass empirically determined) in 20 μl of hybridization buffer composed of 50% deionized formamide, 5×SSPE, 1× Denhardt's solution, 50 μg/ml Yeast tRNA, 50 μg/ml denatured Salmon sperm DNA, 10% Dextran sulphate, 0.2% CHAPS and Molecular Grade water. Slides were placed in a sealed moist chamber (50% deionized formamide/5×SSPE) and incubated overnight at 55° C., 58° C. or 60° C. depending on the experimental set up. The following day the slides were washed with two to three washes of 0.2×SSPE/0.05% Saponin pre-warmed to either at 55° C., 58° C. or 60° C. (10 minutes each), then with two to three washes of 0.1×SSPE/0.05% Saponin pre-warmed to either at 55° C., 58° C. or 60° C. (10 minutes each), then incubated with 0.2×SSPE (10 minutes at room temperature), and then rinsed in 1×blocking buffer (Fluorescent Antibody Enhancer Set for DIG Detection, Roche Molecular Biochemicals, Cat#1768506) for 30 minutes at room temperature. In some cases, a third wash set of of 0.05×SSPE/0.05% Saponin pre-warmed to either at 55° C., 58° C. or 60° C. (10 minutes each) was done. The temperature of the washes was performed at either the temperature of the hybridization or higher; up to 60° C. and with some agitation. Temperatures greater than 60° C. resulted in poor morphology and impaired data analysis. The Digoxigenin label on the bound riboprobe was detected using two rounds of the first two reagents from Fluorescent Antibody Enhancer Set for DIG Detection, Roche Molecular Biochemicals, Cat#1768506 according to manufacturer's instructions, then followed by a sheep anti-DIG Fab labeled with Horseradish peroxidase (Roche Molecular Biochemicals, Cat#1207733) diluted 1:500 in PBS. Finally, TSA™-Fluorescein (NEN™ Life Science Products, Inc., Boston, Mass.) was added as a substrate for HRP resulting in activation and covalent deposition of Fluorescein-tyramide according to manufacturer's instructions. After washing the slides in PBS, the cells were counter stained with 4′,6-Diamidino-2-phenylindole (DAPI, Molecular Probes, Inc., Eugene, Oreg.; Cat #D-1306). Slides were washed twice in PBS and once in Molecular Grade water, dried in 38° C. oven and mounted with ProLong™ mounting medium (Molecular Probes, Inc., Eugene, Oreg.; Cat #P7481). Slides were stored in the dark at 4° C. until fluorescent microscopic analysis. Cellular epifluorescent signal was visualized with a stationary multi-band beamsplitter and emitter mounted in the body of a Zeiss Axioskop microscope (Zeiss; Thornwood, NY) with single and multi-band excitation filters fitted in a lud1 filter wheel (Lud1; Hawthorne, N.Y.) using Chroma's 83000 filter set (Chroma; Brattleboro, Vt.), an AttoArc power source (Zeiss; Thornwood, N.Y.), a charged coupled device (Photometrics, Tucson, Ariz., Model SenSys™) and QUIPS™ SmartCapture™ image capture software, Version 3.1.2 (Vysis, Inc., Downer's Grove, Ill.) resident on a Macintosh Power PC G3/400 (Apple Computer; Cupertino, Calif.).

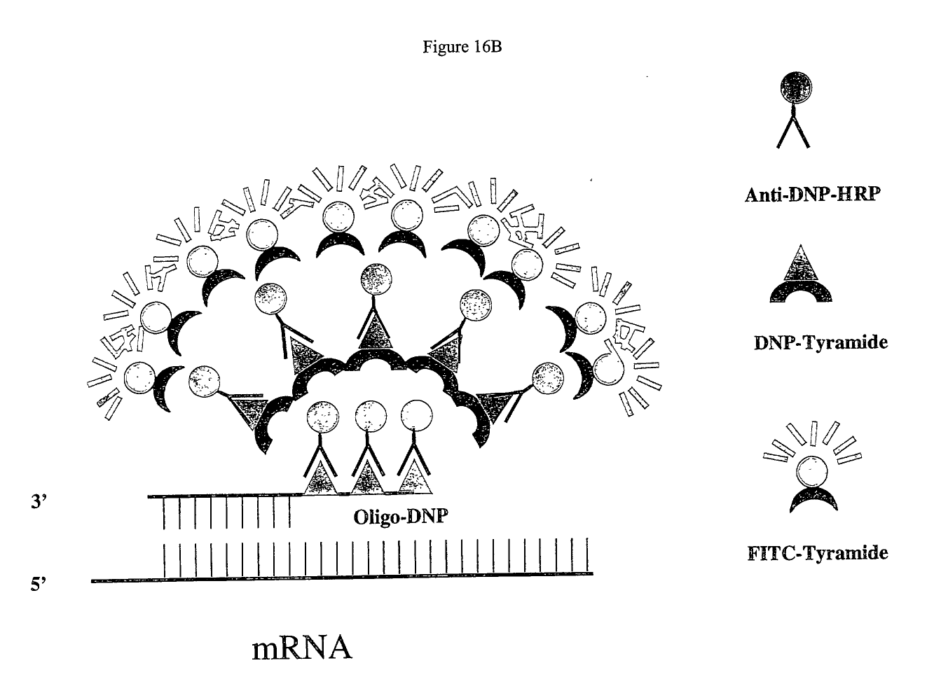

The in situ data illustrated in FIGS. 9-16 show the results of each tag (probe) with multiple preparations (6-7 each) of fetal erythroblasts and adult erythroblasts. For all the tags, green signal (fluorescein) representing specific hybridization of the probe is seen in the cytoplasm of the fetal erythroblast cells to a higher degree than in the adult erythroblasts. For orientation, the nucleus is counter stained blue with the nuclear dye, DAPI. For direct comparison of signals in fetal erythroblasts vs. adult erythroblasts and adult peripheral white cells, multiple sets of these tissue types were performed in the same experiment. They were all treated identically. To create these figures, the comparable images were pulled from the server and a screen shot was captured for the images shown for each tag (probe). The adult nucleated white blood cells done in the same experiments were all negative for signal in the cytoplasm and the data is not shown.

### 7.3. Example 3

### Full Length cDNA Library Screening

Since the nine tags detailed above showed fetal erythroid specificity and that the tags identified from our subtracted cDNA libraries represent only fragments (Rsa I digested short fragments) of the entire mRNA, full length libraries were screened to pull the full length mRNA for each of the nine tags.

Full length cDNA library construction: The construction of the full-length cDNA libraries were performed according to manufacturer's instructions. cDNA synthesis from mRNA greater than 5 μg was made by using ZAP-cDNA ® Synthesis Kit (Cat #200400, Stratagene, La Jolla, Calif.). From total or mRNA less than lug, the cDNA synthesis was performed with CapFinder™ PCR cDNA library construction kit (Cat#K1051-1) from Clontech Laboratories, Inc. Palo Alto, Calif. according to manufacturer's instructions except for the following modifications. After second strand cDNA synthesis, cDNA was size selected by low melting agarose gel-electrophoresis, followed by phenol/chloroform extraction. The cDNA was then ethanol precipitated, washed and resuspended in water and ligated to EcoRI digested and CIAP-treated lambda ZAPS II vector (Cat #236211, Stratagene, La Jolla, Calif.). The ligated cDNA was packaged and used to infect E. coli XL-1 Blue MRF′. The titer of each library was more than 5×10⁶ plaque forming units (pfu). cDNA libraries were then amplified by either PCR or phage infection into bacteria.

Full length screening by plaque hybridization: The short-fragment tags from the subtracted cDNA libraries were used as probes to isolated full-length sequences of the tags. The procedure was according to Quertermous, T., 1996, in Current Protocols in Molecular Biology, Ausubel, F. M., et. al., Eds, John Wiley & Sons, Inc., New York, N.Y., pp. 6.1.1-6.1.4. Phagemid containing cDNA inserts were excised from lambda ZAP® II vector (Cat #236211, Stratagene, La Jolla, Calif.) by in vivo excision. The clones containing the longest cDNA inserts were sequenced and compared using the BLAST2 algorithm (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs,” Nucleic Acids Res. 25:3389-3402), to GenBank (release 117; NCBI, Bethesda, Md.) EMBL (release 62; EBI, Cambridge, UK), human EST (release 117, NCBI, Bethesda, Md.), LifeSeq® Gold full length and component sequences Version 5.1, May 2000 release (Incyte Genomics, Palo Alto, Calif.) for DNA identity.

The following table shows the relationship between the tags, their full-length genes and their respective lengths in base pairs (bp).

TABLE 4

| Original | SIZE of | SEQ. | | SIZE of FL | SEQ. |
|---|---|---|---|---|---|
| Tag | Tag (bp) | ID NO. | Name of FL | (bp) | ID NO. |
| 1503-7E | 711 | 10 | J42-4d | 3194 | 11 |
| 305-4G | 378 | 15 | K1-1a | 2256 | 16 |
| 597-10C | 1068 | 21 | NT7-T3 | 2186 | 22 |
| 334-2C | 1159 | 24 | O19r-T3 | 3561 | 25 |
| 332-9E | 1126 | 27 | P60-1a | 3215 | 28 |
| 305-9E | 1014 | 31 | R5′-T3 | 3230 | 32 |
| 369-8G | 454 | 34 | U2f-T3 | 3661 | 35 |
| 305-6G | 1095 | 36 | L15-1a | 2103 | 37 |

### 7.4. Example 4

### Identification of Alternatively Spliced Forms of Tag-Related Full-Length Genes

The initial purpose of this work was to confirm that the full-length genes identified by plaque hybridization contained their entire 5′ and 3′ ends. As a function of this work, alternatively spliced forms were found for almost all of the eight tag-related full length gene sequences. Some were only alternative polyadenylation sites so are not mentioned here. However, others that reflect sequence variation are included and are referred to as tag-related splice variants. Since the variant regions may be even more tissue specific and useful for designing cellular identification probes, we decided to extensively study the alternatively spliced forms for each of the tag-related full-length genes. 5′ and 3′ RACE (rapid amplification of cDNA ends) was conducted using a SMART™ RACE cDNA Amplification Kit (Cat#K1811-1) from Clontech Laboratories, Inc. Palo Alto, Calif. according to manufacturer's instructions. PCR products were cloned into PCR-Script™ (SK+) vector (Cat #211189, Stratagene, La Jolla, Calif.) and sequenced with T3 and T7 primers using Dye-Terminator chemistry on a 377 ABI fluorescent DNA sequencer (PE Biosystems, Inc. Foster City, Calif.). Sequences were then compared with tags and tag-related full length genes by using Sequencher 3.1 and 4.0 softwares (Gene Codes Corp, Ann Arbor, Mich.). For tag 305-4G (SEQ. ID NO.15), 334-2C (SEQ. ID NO.24), 332-9E (SEQ. ID NO. 27) and 305-6G (SEQ. ID NO. 36) we also used plaque hybridization and PCR to increase the likelihood for obtaining variants.

The tags, and the respective tag-related full length genes and tag-related splice variants are listed in the table below.

TABLE 5

| | | | | | Method used for |
|---|---|---|---|---|---|
| | | SEQ. | Name of | SEQ | obtaining |
| Name | Name of | ID | alternative | ID | alternative |
| Tag | FL | NO. | spliced form | NO. | spliced form |
| 1503-7E | J42-4d | 11 | J2r(3) | 12 | 5′ RACE |
| | | | J2r(12) | 13 | 5′ RACE |
| | | | J2r(13) | 14 | 5′ RACE |
| 305-4G | K1-1a | 16 | K2r/1f(50) | 17 | PCR |
| | | | K2r/1f(59) | 18 | PCR |
| | | | K(1)157-2A | 19 | plaque |
| | | | | | hybridization |
| | | | K3r(HIGH)76 | 20 | 3′ RACE |
| 597-10C | NT7-T3 | 22 | N9r/Mf | 23 | 5′ RACE |
| 334-2C | O19r-T3 | 25 | O1-1a | 26 | plaque |
| | | | | | hybridization |
| 332-9E | P60-1a | 28 | P1-1a | 29 | plaque |
| | | | | | hybridization |
| | | | P3r(9) | 30 | 5′ RACE |
| 305-9E | R5′-T3 | 32 | R6r/1-6H | 33 | 5′ RACE |
| 369-8G | U2f-T3 | 35 | — | | — |
| 305-6G | L15-1a | 37 | — | | — |

All tags, tag-related full length genes and tag-related splice variants can be used for the purposes of this invention. Specifically, all of these nucleic acid sequences are useful to distinguish fetal cells from maternal cells.

### 8. EXAMPLE

### Detection of Tag Sequences by in situ Hybridization

Dioxygenin (DIG)-labeled riboprobes corresponding to tags identified in the screening methods of Section 7, supra (SEQ ID NOs:10, 15, 21, 24, 27, 31, 34, 36 and 41) were synthesized and titered on fetal liver blood and adult peripheral blood. After validation of each of the probes' reactivity in the cytoplasm of fetal liver erythroblast and not in adult nucleated peripheral blood cells, the probes were tested in other relevant tissues, such as circulating fetal erythroblasts (fetal cord blood pools from 8-12 week gestation human fetuses), adult erythroblast (adult bone marrow from iliac crest enriched for erythroblast using an anti-transferrin receptor antibody attached to a solid phase, which resulted in bone marrow preparations that were ˜90% erythroid compared to 20-35% prior to enrichment.) and adult nucleated peripheral blood cells (white cell fraction from whole adult blood). Experiments were set up to test all these population with each probe, beginning with hybridization conditions of lower stringency and moving to higher stringency conditions. This was accomplished by varying experimental parameters such as: the temperature of hybridization (from 55° C. to 60° C.), the temperature of the washes (from 55° C. to 60° C.), the salt concentration in the washes (0.2×SSPE to 0.05×SSPE), the number of washes of each type (two-three times), and finally, the addition of 100 mM TMAC to the hybridization, wash and moist chamber buffers.

### 8.1. Materials and Methods

Mononuclear fractions of adult bone marrow, peripheral blood and washed 8-12 week fetal cord blood were prepared according to standard cell biology methods (Celis, 1998, “Cell Biology, A Laboratory Handbook”, 2nd Ed., Academic Press, San Diego, Calif.). The in situ hybridization method used was based on Rosen B. and Beddington R. (Rosen and Beddington, 1994, Detection of mRNA in whole mounts of mouse embryos using digoxigenin riboprobes. In “Methods in Molecular Biology” (Isaac, P. G., Ed), Vol 28, pp. 201-208, Humana Press Inc., Totowa, N.J.) with some modifications. Paraformaldehyde (4%)/acetic acid (5%) fixed cells on slides were permeabilized with Proteinase K (0.1 mg/ml in PBS) for 10 minutes at room temperature. Cells were then post fixed with 4% paraformaldehyde/5% acetic acid for 5 minutes and incubated in 0.05% Saponin/PBS twice for 10 minutes each. Hydrogen peroxidase activity was blocked using 3% Hydrogen peroxide/1% Sodium azide/PBS for 40 minutes. Digoxigenin labeled riboprobes were synthesized using a method based on both Signer's protocol (Signer, 1994, Digoxigenin labeling of RNA transcripts from multi- and single-locus DNA minisatellite probes. In “Methods in Molecular Biology” (Isaac, P. G., Ed), Vol 28, pp. 77-81, Humana Press Inc., Totowa, N.J.) and controlled alkali hydrolysis at 65° C. (Anderson, 1999 “Nucleic Acid Hybridization” (Rickwood, D., Ed.) p.125, Springer-Verlag New York Inc., New York). In the analyses the average hydrolyzed non-radioactive riboprobe was targeted to be between 150 and 200 nucleotides in length. In addition, riboprobes synthesized using Digoxigenin-1-UTP (Roche Molecular Biochemicals) on average contain one Digoxigenin-11-UTP every twenty nucleotides resulting in an average of 7 digoxigenin labels per 150 nucleotides to 10 digoxigenin labels per 200 nucleotides of hydrolyzed non-radioactive riboprobe fragment.

Slides were incubated overnight at either 55° C., 58° C. or 60° C. with a mixture of 20 ml of each riboprobe in hybridization buffer (50% deionized formamide, 5×SSPE, 1×Denhardt's solution, 50 mg/ml Yeast tRNA, 50 mg/ml denatured salmon sperm DNA, 10% Dextran sulphate, and 0.2% CHAPS). Slides were washed minimally with 0.2×SSPE/0.05% Saponin three times and 0.1×SSPE/0.05% Saponin twice at the same temperature as the hybridization. Temperature and ionic conditions of the hybridizations and wash steps did not go higher than 60° C. or above 3 times with 0.1×SSPE to preserve cellular morphology.

The digoxigenin labeled riboprobes were detected using two rounds of the first two reagents from the Fluorescent Antibody Enhancer Set for DIG Detection (Roche Molecular Biochemicals), namely a mouse IgG₁ monoclonal antibody and a digoxigenin labeled anti-mouse IgG F(ab′)₂ fragment, followed by incubation with a sheep anti-DIG horseradish peroxidase labeled Fab (Roche Molecular Biochemicals) and the HRP substrate TSA™-Fluorescein (NEN™ Life Science Products, Inc., Boston, Mass.). After washing the slides with PBS the cells were counter stained with 4′,6-Diamidino-2-phenylindole (DAPI), washed again with PBS, followed by water, then dried and mounted with ProLong™ mounting medium (Molecular Probes, Inc., Eugene). Cellular epifluorescent signal was visualized with a stationary multi-band beamsplitter and emitter mounted in the body of a Zeiss Axioskop epifluorescence microscope (Zeiss; Thornwood, N.Y.) with single and multi-band excitation filters fitted in a Ludl filter wheel (Ludl; Hawthorne, N.Y.) using Chroma's 83000 filter set (Brattleboro, Vt.) and equipped with a CCD camera (Photometrics, Tucson, Ariz., ModelSenSys™). Images were captured using QUIPS™ SmartCapture™ image capture software, Version 3.1.2 (Vysis, Inc., Downer's Grove, Ill.).

### 8.2. Results

Using the above visualization and detection systems cells were determined to be positive for riboprobe in situ hybridization analysis when the FITC signal (green) at least two fold greater than background and the cell contained a nucleus (blue). Negative cells were cells that either lacked FITC signal (green) within the cellular membrane borders (red) and/or the cell lacked a nucleus (blue). Thus captured images of cells positive for the non-radioactive riboprobe would have within its cellular membranes a nucleus that was labeled blue and peroxidase-antibody cascades that were labeled green.

By assessing the hybridization tissues across many conditions, the tags of SEQ ID NOs:10, 15, 21, 24, 27, 31, 34, 36 and 41 were shown to selectively or specifically hybridize to erythroblasts, as the hybridization signal varied in a predictable manner with the stringency of hybridization. While tag 252 (SEQ ID NO:39) was not found in adult peripheral blood cells, its expression level in adult and fetal erythroblasts was indistinguishable.

### 9. SPECIFIC EMBODIMENTS, CITATION OF REFERENCES

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references, including patent applications, patents, and scientific publications, are cited herein; the disclosure of each such reference is hereby incorporated herein by reference in its entirety.

What is claimed is:

1. A method for detecting a fetal cell in a maternal blood sample, comprising the steps of:
(a) contacting said maternal blood sample with a first probe, said first probe comprising:
(i) a nucleotide sequence corresponding to SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42,
(ii) a nucleotide sequence having at least 90% sequence identity to at least 20 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or
(iii) a nucleotide sequence having at least 80% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42,
which first probe selectively or specifically hybridizes to mRNA in fetal cells if present in the maternal blood sample; and
(b) identifying whether or not said maternal blood sample comprises a cell that comprises mRNA that detectably hybridizes to the first probe, thereby detecting whether or not said maternal blood sample contains a fetal cell.

2. The method of claim 1, wherein the first probe specifically hybridizes to fetal cells.

3. The method of claim 1, wherein the first probe selectively hybridizes to fetal cells.

4. The method of claim 1 in which the first probe comprises a label.

5. The method of claim 4 in which the label is a radioactive label, a fluorescent label, a colorimetric reagent, or an enzyme.

6. The method of claim 1, wherein the nucleotide sequence is 20-30 nucleotides in length.

7. The method of claim 1, wherein the nucleotide sequence is 30-40 nucleotides in length.

8. The method of claim 1, wherein the nucleotide sequence is 40-60 nucleotides in length.

9. The method of claim 1, wherein the nucleotide sequence is 60-80 nucleotides in length.

10. The method of claim 1, wherein the nucleotide sequence is 80-100 nucleotides in length.

11. The method of claim 1, wherein the nucleotide sequence is 100-150 nucleotides in length.

12. The method of claim 1, wherein the nucleotide sequence is 150-200 nucleotides in length.

13. The method of claim 1, wherein the nucleotide sequence is greater than 200 nucleotides in length.

14. The method of claim 1, wherein the probe is less than 40 nucleotides in length.

15. The method of claim 1, wherein the probe is less than 50 nucleotides in length.

16. The method of claim 1, wherein the probe is less than 100 nucleotides in length.

17. The method of claim 1, wherein the probe is less than 200 nucleotides in length.

18. The method of claim 1, wherein the probe is less than 300 nucleotides in length.

19. The method of claim 1, wherein the probe is less than 400 nucleotides in length.

20. The method of claim 1, wherein the probe is less than 500 nucleotides in length.

21. The method of claim 1, wherein the probe is less than 1,000 nucleotides in length.

22. The method of claim 1, wherein the probe is less than 2,000 nucleotides in length.

23. The method of claim 1, further comprising, prior to step (b), contacting said maternal blood sample with a second probe which selectively or specifically hybridizes to fetal cells if present in the maternal blood sample.

24. The method of claim 23, further comprising detecting a cell in said maternal blood sample which comprises mRNA that hybridizes to the second probe.

25. The method of claim 24, wherein the first and second probe are labeled with the same type of label.

26. The method of claim 23, wherein the first and second probes correspond to the same mRNA.

27. The method of claim 23, wherein the first and second probes correspond to different mRNAs.

28. The method of claim 1, further comprising, prior to step (a), immunoenriching the maternal blood sample for fetal cells.

29. The method of claim 28, wherein immunoenriching the maternal blood sample comprises:
(a) contacting the maternal blood sample with an antibody that selectively or specifically binds to fetal cells in the maternal blood sample; and
(b) separating cells in the maternal blood sample that bind to the antibody from cells that do not bind to the antibody, thereby immunoenriching the maternal blood sample for fetal cells.

30. The method of claim 28, wherein immunoenriching the maternal blood sample comprises:
(a) contacting the maternal blood sample with an antibody that selectively or specifically binds to maternal cells in the maternal blood sample; and
(b) separating cells in the maternal blood sample that do not bind to the antibody from cells that bind to the antibody, thereby immunoenriching the maternal blood sample for fetal cells.

31. The method of claim 1, wherein the probe is an RNA probe.

32. The method of claim 1, wherein the probe is a DNA probe.

33. The method of claim 1, wherein the fetal cell is an erythroblast.

34. The method of claim 1, wherein the fetal cell is a trophoblast.

35. The method of claim 1, wherein nucleotide sequence has at least 95% sequenceidentity to at least 20 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

36. The method of claim 35, wherein nucleotide sequence has 100% sequence identity to at least 20 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

37. The method of claim 1, wherein nucleotide sequence has at least 85% sequence identity to at least 40 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

38. The method of claim 37, wherein the nucleotide sequence has at least 90% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

39. The method of claim 38, wherein the nucleotide sequence has at least 95% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

40. The method of claim 39, wherein the nucleotide sequence has 100% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

41. A method for diagnosing an abnormality in a fetal cell, comprising the steps of:
(a) contacting a maternal blood sample with a first probe, said first probe comprising:
(i) a nucleotide sequence corresponding to SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42,
(ii) a nucleotide sequence having at least 90% sequence identity to at least 20 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or
(iii) a nucleotide sequence having at least 80% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42,
which first probe selectively or specifically hybridizes to mRNA in a fetal cell if present in the maternal blood sample;
(b) identifying whether or not said maternal blood sample comprises a cell that comprises mRNA that detectably hybridizes to the first probe, thereby detecting whether or not said maternal blood sample contains a fetal cell; and
(c) if a fetal cell is detected, determining whether the abnormality exists in said fetal cell, thereby diagnosing the abnormality.

42. The method of claim 41, wherein the first probe specifically hybridizes to the fetal cell.

43. The method of claim 41, wherein the first probe selectively hybridizes to the fetal cell.

44. The method of claim 41 in which the first probe comprises a label.

45. The method of claim 44 in which the label is a radioactive label, a fluorescent label, a colorimetric reagent or an enzyme.

46. The method of claim 41, wherein the nucleotide sequence is 20-30 nucleotides in length.

47. The method of claim 41, wherein the nucleotide sequence is 30-40 nucleotides in length.

48. The method of claim 41, wherein the nucleotide sequence is 40-60 nucleotides in length.

49. The method of claim 41, wherein the nucleotide sequence is 60-80 nucleotides in length.

50. The method of claim 41, wherein the nucleotide sequence is 80-100 nucleotides in length.

51. The method of claim 41, wherein the nucleotide sequence is 100-150 nucleotides in length.

52. The method of claim 41, wherein the nucleotide sequence is 150-200 nucleotides in length.

53. The method of claim 41, wherein the nucleotide sequence is greater than 200 nucleotides in length.

54. The method of claim 41, wherein the probe is less than 40 nucleotides in length.

55. The method of claim 41, wherein the probe is less than 50 nucleotides in length.

56. The method of claim 41, wherein the probe is less than 100 nucleotides in length.

57. The method of claim 41, wherein the probe is less than 200 nucleotides in length.

58. The method of claim 41, wherein the probe is less than 300 nucleotides in length.

59. The method of claim 41, wherein the probe is less than 400 nucleotides in length.

60. The method of claim 41, wherein the probe is less than 500 nucleotides in length.

61. The method of claim 41, wherein the probe is less than 1,000 nucleotides in length.

62. The method of claim 41, wherein the probe is less than 2,000 nucleotides in length.

63. The method of claim 41, further comprising, prior to step (b), contacting said maternal blood sample with a second probe which selectively or specifically hybridizes to fetal cells if present in the maternal blood sample.

64. The method of claim 63, further comprising detecting a cell in said maternal blood sample which comprises mRNA that hybridizes to the second probe.

65. The method of claim 64, wherein the first and second probe are labeled with the same type of label.

66. The method of claim 63, wherein the first and second probes correspond to the same mRNA.

67. The method of claim 63, wherein the first and second probes correspond to different mRNAs.

68. The method of claim 41, further comprising, prior to step (a), enriching the maternal blood sample for fetal cells prior.

69. The method of claim 68, wherein immunoenriching the maternal blood sample comprises:
(a) contacting the maternal blood sample with an antibody that selectively or specifically binds to fetal cells in the maternal blood sample; and
(b) separating cells in the maternal blood sample that bind to the antibody from cells that do not bind to the antibody, thereby immunoenriching the maternal blood sample for fetal cells.

70. The method of claim 68, wherein immunoenriching the maternal blood sample comprises:
(a) contacting the maternal blood sample with an antibody that selectively or specifically binds to maternal cells in the maternal blood sample; and
(b) separating cells in the maternal blood sample that do not bind to the antibody from cells that bind to the antibody, thereby immunoenriching the maternal blood sample for fetal cells.

71. The method of claim 41, wherein the probe is an RNA probe.

72. The method of claim 41, wherein the probe is a DNA probe.

73. The method of claim 41, wherein nucleotide sequence has at least 95% sequence identity to at least 20 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

74. The method of claim 73, wherein nucleotide sequence has 100% sequence identity to at least 20 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

75. The method of claim 41, wherein nucleotide sequence has at least 85% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

76. The method of claim 75, wherein the nucleotide sequence has at least 90% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

77. The method of claim 76, wherein the nucleotide sequence has at least 95% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

78. The method of claim 77, wherein the nucleotide sequence has 100% sequence identity to at least 40 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

79. The method of claim 41, wherein the abnormality is a chromosomal abnormality.

80. The method of claim 79, wherein the chromosomal abnormality is an aneuploidy.

81. The method of claim 80, wherein the aneuploidy is trisomy 13, trisomy 21, or Klinefelter syndrome.

82. The method of claim 41, wherein the abnormality is a single gene disorder.

83. The method of claim 82, wherein the single gene disorder is a deletion, insertion or substitution disorder.

84. The method of 83, wherein the single gene disorder is spina bifida, sickle-cell anemia, a thalassemia, Marfan Syndrome, Duchenne Muscular Dystrophy, or cystic fibrosis.

85. The method of claim 41, wherein the abnormality is a nucleotide triplet expansion in the gene.

86. The method of claim 85, wherein the gene is the Fragile X Syndrome gene, the Friedreich's ataxia gene, the myotonic dystrophy gene, or the Huntington's disease gene.

87. The method of claim 41, wherein the fetal cell is an erythroblast.

88. The method of claim 41, wherein the fetal cell is a trophoblast.

89. The method of claim 41, wherein determining whether the abnormality exists in said fetal cell comprises the steps of:
(d) contacting the maternal blood sample with a diagnostic probe under conditions that allow hybridization of the diagnostic probe to a diagnostic target sequence in the fetal cell, wherein the manner of hybridization of the diagnostic probe to the diagnostic target sequence is indicative of whether the abnormality exists in the fetal cell; and
(e) determining the manner in which the diagnostic probe hybridizes to the target sequence, thereby determining whether the abnormality exists in the fetal cell.

90. The method of claim 89, wherein steps (a) and (d) are performed simultaneously.

91. The method of claim 90, further comprising, prior to steps (a) and (d), contacting the maternal blood sample with a first fixative, wherein the first fixative comprises 4% formalin and has a pH of 6-8.

92. The method of claim 91, further comprising, following steps (a) and (d), contacting the maternal blood sample with a second fixative following, wherein the second fixative comprises 4% formalin and has a pH of less than 6.

93. A method for identifying a nucleic acid useful as a probe for fetal cells in the maternal circulation, comprising the steps of:
(a) performing differential expression analysis on RNA or cDNA obtained from fetal liver myeloid cells of less than 22 weeks of gestation relative to RNA or cDNA obtained from more mature liver or non-liver myeloid cells; and
(b) identifying an RNA or cDNA species that is selectively or specifically expressed in the fetal liver myeloid cells,
wherein the RNA or cDNA species that is selectively or specifically expressed in the fetal liver myeloid cells is useful as a probe for fetal cells in the maternal circulation.

94. The method of claim 93, wherein the fetal liver is human fetal liver.

95. The method of claim 93, wherein the fetal liver myeloid cells are obtained before 20 weeks of gestation.

96. The method of claim 95, wherein the fetal liver myeloid cells are obtained between 10 and 15 weeks of gestation.

97. The method of claim 95, wherein the more mature myeloid cells are fetal cord blood cells obtained after 22 weeks of gestation, fetal peripheral blood cells obtained after 22 weeks of gestation, or fetal liver myeloid cells obtained after about 22 weeks of gestation.

98. The method of claim 93, wherein the mature myeloid cells are adult bone marrow cells or adult peripheral blood cells.

99. The method of claim 93, wherein the differential expression analysis comprises subtraction suppression hybridization (SSH).

100. A kit comprising in one or more containers
(a) a first probe, said first probe comprising:
(i) a nucleotide sequence corresponding to SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42;
(ii) a nucleotide sequence having at least 90% sequence identity to at least 20 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42; or
(iii) a nucleotide sequence having at least 80% sequence identity to at least 40 consecutive nucleotides of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42,
which first probe selectively or specifically hybridizes to mRNA in fetal cells if present in a maternal blood sample; and
(b) instructions for diagnostic use or a label indicating regulatory approval for diagnostic use.

101. The kit of claim 108, further comprising an antibody that selectively or specifically binds to fetal cells in a maternal blood sample.

102. The kit of claim 108, further comprising a second probe that selectively or specifically hybridizes to mRNA in fetal cells if present in a maternal blood sample.

103. The kit of claim 102, wherein the second probe comprises:
(a) a nucleotide sequence corresponding to SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42;
(b) a nucleotide sequence having at least 90% sequence identity to at least 20 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42; or
(c) a nucleotide sequence having at least 80% sequence identity to 40 consecutive nucleotides of SEQ ID NO:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42.

104. The kit of claim 102, wherein the first probe and the second probe correspond to the same mRNA.

105. The kit of claim 102, wherein the first probe and the second probe correspond to different mRNAs.

106. The kit of claim 102, wherein the first probe and the second probe are labeled with the same type of label.

107. A method for detecting a fetal cell in a maternal blood sample, comprising the steps of:
(a) performing differential expression analysis on RNA or cDNA obtained from fetal liver myeloid cells relative to RNA or cDNA obtained from mature myeloid cells;
(b) identifying an RNA or cDNA species that is selectively or specifically expressed in the fetal liver myeloid cells, thereby identifying an RNA or cDNA species that is useful as a probe for fetal cells in the maternal circulation;
(c) contacting the maternal blood sample with a probe comprising a nucleotide sequence corresponding to all or a portion of the RNA or cDNA of step (b); and
(d) identifying whether or not said maternal blood sample comprises a cell that comprises mRNA that detectably hybridizes to the first probe, thereby detecting whether or not said maternal blood sample contains a fetal cell.

108. A method for diagnosing an abnormality in a fetal cell, comprising the steps of:
(a) performing differential expression analysis on RNA or cDNA obtained from fetal liver myeloid cells relative to RNA or cDNA obtained from mature myeloid cells;
(b) identifying an RNA or cDNA species that is selectively or specifically expressed in the fetal liver myeloid cells, thereby identifying an RNA or cDNA species that is useful as a probe for fetal cells in the maternal circulation;
(c) contacting the maternal blood sample with a probe comprising a nucleotide sequence corresponding to all or a portion of the RNA or cDNA of step (b);
(d) identifying whether or not said maternal blood sample comprises a cell that comprises mRNA that detectably hybridizes to the first probe, thereby detecting whether or not said maternal blood sample contains a fetal cell; and
(e) if the maternal blood sample contains a fetal cell, determining whether the abnormality exists in said fetal cell, thereby diagnosing the abnormality.

109. An isolated nucleic acid molecule selected from the group consisting of
(a) a nucleic acid molecule having a nucleotide sequence which is at least 90% identical to the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof;
(b) a nucleic acid molecule comprising at least 15 nucleotide residues and having a nucleotide sequence identical to at least 15 consecutive nucleotide residues of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof,
(c) a nucleic acid molecule which encodes a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78;
(d) a nucleic acid molecule which encodes a fragment at least 10 consecutive amino acid residues of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78;
(e) a nucleic acid molecule which encodes a fragment of a polypeptide comprising the amino acid sequence of any SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78; wherein the fragment comprises consecutive amino acid residues corresponding to at least half of the full length of any of said SEQ ID NOs; and
(f) a nucleic acid molecule which encodes a naturally occurring allelic variant of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78, wherein the nucleic acid molecule hybridizes with a nucleic acid molecule consisting of the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42 under stringent conditions, or a complement thereof.

110. The isolated nucleic acid molecule of claim 109, which is selected from the group consisting of:
(a) a nucleic acid having the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof; and
(b) a nucleic acid molecule which encodes a polypeptide having the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78, or a complement thereof.

111. The nucleic acid molecule of claim 109, further comprising a vector nucleic acid sequence.

112. The nucleic acid molecule of claim 109, further comprising a nucleic acid sequence encoding a heterologous polypeptide.

113. A host cell which contains the nucleic acid molecule of claim 109.

114. The host cell of claim 113 which is a mammalian host cell.

115. A non-human mammalian host cell containing the nucleic acid molecule of claim 109.

116. An isolated polypeptide selected from the group consisting of:
(a) a fragment of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78;
(b) a naturally occurring allelic variant of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78, wherein the polypeptide is encoded by a nucleic acid molecule which hybridizes with a nucleic acid molecule consisting of the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42 under stringent conditions, or a complement thereof; and
(c) a polypeptide which is encoded by a nucleic acid molecule comprising a nucleotide sequence which is at least 90% identical to a nucleic acid consisting of the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof.

117. The isolated polypeptide of claim 116 having the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78.

118. The polypeptide of claim 116, wherein the amino acid sequence of the polypeptide further comprises heterologous amino acid residues.

119. An antibody which selectively binds with the polypeptide of claim 116.

120. A method for producing a polypeptide selected from the group consisting of:
(a) a polypeptide comprising the amino acid sequence of any of SEQ ID NOs: 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78;
(b) a polypeptide comprising a fragment of at least 10 contiguous amino acids of the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78; and
(c) a naturally occurring allelic variant of a polypeptide comprising the amino acid sequence of any of SEQ ID NOs:43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, or 78, wherein the polypeptide is encoded by a nucleic acid molecule which hybridizes with a nucleic acid molecule consisting of the nucleotide sequence of any of SEQ ID NOs:10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41 or 42, or a complement thereof under stringent conditions;
the method comprising culturing the host cell of claim 113 under conditions in which the nucleic acid molecule is expressed.


SEQUENCE LISTING (78 sequences)

SEQ ID NO: 1 - DNA, 48 bp, Homo sapiens
gagtcattctcgacttgcggccgcacgtttgatttccascttggtccc

SEQ ID NO: 2 - DNA, 48 bp, Homo sapiens
gagtcattctcgacttgcggccgcacctaggacggtcagcttggtccc

SEQ ID NO: 3 - DNA, 24 bp, Homo sapiens
gcgatggttgttgtcattgtcggc

SEQ ID NO: 4 - DNA, 17 bp, Homo sapiens
caggaaacagctatgac

SEQ ID NO: 5 - DNA, 21 bp, Homo sapiens
ggtgctcttggaggagggtgc

SEQ ID NO: 6 - DNA, 18 bp, Homo sapiens
caggaaacaagctatgac

SEQ ID NO: 7 - DNA, 17 bp, Homo sapiens
gaattttctgtatgagg

SEQ ID NO: 8 - DNA, 678 bp, Homo sapiens
caggtgaagctgcagcagtcaggggctgaactggtgcagcctggggcttcagtgaatttgtcctgcaaggcctctggcttcaccctcaccagctactatatgtactggttgaagcagaggcctggacaaggccttgagtggatcggagagatcaaccctagcaatggtgttactaattttaatgagaagttcaagagcaaggccacactgactgtagacaagtcctccagcacagcatacatgctactcagcagcctgacatctgaggactctgcggtctattactgtacaagatcacattactacggccactgggatgttatggactactggggccaagggaccacggtcaccgtcaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 9 - DNA, 648 bp, Homo sapiens
gacatcgagctcactcagtctccaacagtcatgtctgcatctccaggggagaaggtcaccataacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccatgtacacgttcggaggggggacaaagttggaaataaaacggggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - DNA, 711 bp, Homo sapiens
ggggggtttgttgggctggtgagttttcgaggtgacggtgctgtgctcggtgaggggacgcccagagagccctgacccggggtcactccgtcgccgttctcctcttgtctacgtgctggacccggtgctacctttttacccacacttaagtgacgcaaaatgcccttcaatggcgagaagcagtgtgtgggagaggaccagccaagcgattctgattcttcccggttttccgaaagcatggcttcgctcagtgactatgaatgctccaggcagagctttacaagtgactcctccagcaaatccagctctcctgcttcaacaagccctccaagggttgtaacatttgatgaagtgatggctacagcaaggaacttatcaaacttgactcttgctcatgagattgctgtaaatgagaaccttcaattgaaacaagaggctctcccagaaaagagtttggctggtcgagtgaagcacattgttcaccaggccttctgggacgtcttggattcagaactaaatgctgaccctcctgagattgaacatgccatcaaactgtttgaagaaatcagagagattcttctctcttttctcactcccggtggcaaccggcttcgcaaccaaatctgtgaagttttggacacagacctcattaggcagcaggctgagcacagtgctgttgacatccaaggcctggccaactatgtcatcagt

SEQ ID NO: 11 - DNA, 3194 bp, Homo sapiens
ggggggtttgttgggctggtgagttttcgaggtgactgtgctgtgctcggtgaggggacgcccagagagccctgacccggggtcactccgtcgccgttctcctcttgtctacgtgctggacccggtgctacctttttacccacacttaagtgacgcaaaatgcccttcaatggcgagaagcagtgtgtgggagaggaccagccaagcgattctgattcttcccggttttccgaaagcatggcttcgctcagtgactatgaatgctccaggcagagctttacaagtgactcctccagcaaatccagctctcctgcttcaacaagccctccaagggttgtaacatttgatgaagtgatggctacagcaaggaacttatcaaacttgactcttgctcatgagattgctgtaaatgagaactttcaattgaaacaagaggctctcccagaaaagagtttggctggtcgagtgaagcacattgttcaccaggccttctgggacgtcttggattcagaactaaatgctgaccctcctgagtttgaacatgccatcaaactgtttgaagaaatcagagagattcttctctcttttctcactcccggtggcaaccggcttcgcaaccaaatctgtgaagttttggacacagacctcattaggcagcaggctgagcacagtgctgttgacatccaaggcctggccaactatgtcatcagtacgatgggaaagctgtgtgctcccgtgcgagataatgatatcagagagttaaaggctactggcaacatcgtggaggtgctgagacaaatattccatgtcctggacctcatgcaaatggacatggccaattttacaattatgagtctcagaccgcaccttcaacgccagttggtggaatatgagagaaccaagttccaggaaattttggaagaaactccaagtgagtataatataatgtgtatttatattgaaattaggttaaaatgatgattttaaatgttaaaatttaaattaaattaaattaaattaaattaataaataaataaaatttaatttaatttaaattaattaaaatttaaaatgttaaaggttaaaatgaaaattaggtgatgttttcatttttgacttcatatatagtatcactaaagaattttggcctttctattaaacatttttttaccttccaaaattgacatcacaaacttacagttgtttattctgtccaggaactgtgctaagcactattcgtttcttcattcattcaagatatatttattgagtgcctcctgtgtttcagacactgcttaggtgcagtggatactgcagcgaacagaatggaatacagttgccgtccccaaggagctgacattctagtaggcatgacaaataaataaacatatattgtcaggtgtattattaagtggtatgaagaaaaataggataaagggacagtaatgggtcagaagtgaggacagcattgttttttagatgtgatggactgggaaggtctctgtaagtaacatttgagtaaagtttgcaaagaaatgaggaaggggcatgtaaagatggggaaaagaacgttctagcctgagggactgcaggtacaaaggcactgaagcagaaactgcttatgagtttgacaaacacagggcagacattgtgacgggagtagagttggtaaggggagagtggtaagagatgaggctgaaaaggagataaaggcaggtcacatagagcattgttagtaaatgagaggagtttggatttcattccatgtgcggtggaaactcatcagagagttttaatcaggggagcaacatctgattttcatttttgaagaatattcttgttcctggctacagaataggtcatgtgggaacaaagagggaaacagaggtaccaattaggtggcattacagttgctcaggcaagaaatgatggtggcttgttccagatggtaatggtggaaggagtgagaagagaccagattctggatacattttgaaagtggagtaatcagatttgctgaagattggataagggagaaaagagttgagggtgactctaaggtttttggcctcgtacaaccgggtgaatggtcatacagtttgagtgaaactggtgagaccaggagaaagaaggttgggagatgacatggaatcaagagttcagtttggtgatgtaaacttttgagatttcatttgacatcaatgtggagatgtcaggtaggcatttagatatgaattacgagtttacaggaataatggaaactgtatatgtatttgggaatcatcagtatatgtatgatatataaagccatggaactgggtactagttaacacttagaaaatccttatcacactctaagtactttacctgtacaaactaatttaatccttgcaacaactatatgaagttaccccccattttatgggtgcggaaactgaggcactgtgaggttaagtcactggcctatgtaacacacttaaaagtataggagctcacctaggaaggcagcttggctccatagtccatgttcctgtccattatattacactgggtgagactacctagggggagtgagtgtggactctgaaagaaactgacgtggggcactccagcagctagaagctgaagttgagatgaggaggagggtctaacaatggagactatgagggagtagccagagaggtagtgtgacagtcaggagaggcgggtgtcctgtaagatacgtgaagaaggtatttggagaaggaaggcatgtttggatttgacaagcattgctaagtggtcaagaaagacacaggccaacttgaccattggatttgactgtttgatgatggtcagtaaccttgatgagagtggtttcagtggatttgtgtgcattaaagccagactgggcagggcacagtggctcacacctgtaatcctagcactttgggaggccgaggcaggcacatcacctgagatcaggagttcaagaccagcctggccaacatgctgaaaccccttctctactaaaatctaaaaattagccgggatgatggcaggtgcctgtaatcccagctactcgggaggttgagatgggagaatcgcttgaacccaggagatggtggttgcagtgagccaaggtcgcatcattgcactccagcctggggggctgagcaagactccgtctcggaaaaaaaaaaaaaaaaaaaaaa

SEQ ID NO: 12 - DNA, 827 bp, Homo sapiens
gggtgaggtcgtgcagactgctgctgcgtccccttgccgggacctttggttaaggggaagaggtggagaaaagccgattgggcctgggctcgaacgctgaaccctcaggtgttgcttgtgcttcccaaagctgctactgtaggtggcaccactgggggtaatagcaatgatttgaggtggcatatggatgaacatagttacttgttttcgttactattaagaaaatctgttccattaagtaagcagtttcacaatgatagagtttccttttaaaataaatatatttaaatgcaaaaatctagtcaagatagagacgcggttactaaattatagtaaaggtaaggagggagacgtagaaatagtcatgaagagtggcactcaaacgacttaagtttgggaaatcttagatggttgtcctgctaccttaactgtgaagaaactggacaagttgtccttgtccccagtgggtgtctcagcacccatggtcaccagcccagtcagggactttgctcttcttacctgccaacggataatgagttaatctcagtggttcccacttgaggaggctccctgccgtggtccttttcctctcttctctagggatttcatgcaccaccctgacagccagtggaagttgcctgatttgaagaggcccagggccgaagatacccagtcatgtgtggaagcagcttcaatgccattttgtgagaggttgagggccccagagggggaagcacaggtgctacctttttacccacacttaagtgacgcaaaatgcccttcaatggcgagaagcagtgtgtgggagaggaccagccaagcgattc

SEQ ID NO: 13 - DNA, 153 bp, Homo sapiens
acgcacgccaagtcttcagagggcctggatgggtgtgactccaaggagctaatcgtcccgtgcaggtgctacctttttacccacacttaagtgacgcaaaatgcccttcaatggcgagaagcagtgtgtgggagaggaccagccaagcgattc

SEQ ID NO: 14 - DNA, 544 bp, Homo sapiens
tggcactcaaacgacttaagtttgggaaatcttagatggttgtcctgctaccttaactgtgaagaaactggacaagttgtccttgtccccagtgggtgtctcagcacccatggtcaccagcccagtcagggactttgctcttcttacctgccaacggatgatgagttaatctcagtggttcccacttgaggaggctccctgccgtggtccttttcctctcttctctagggatttcatgcatcaccctgacagccagtggaagttgcctgatttgaagaggcccagggccgaagatacccagtcatgtgtggaagcagcttcaatgccattttgtgagaggttgagggccccagagggggaagcacagtgacctgctatcgtgttggaggctttgcttggagtcatcttcagtcttttctgttggctttacctttgaccagtgattaaatcctccaggtgctacctttttacccacacttaagtgacgcaaaatgcccttcaatggcgagaagcagtgtgtgggagaggaccagccaagcgattc

SEQ ID NO: 15 - DNA, 378 bp, Homo sapiens
ctcgtgggggggcgcgcgattatttgaagacgctcacggagcggctggctaggctgaggagagctcgccgggctctgaggcgcaggaattcaataaagaaaatggcagctcttactccaaggaagaggaagcaggattctttgaagtgtgacagccttttacacttcactgaaaatctgtttccatcacctaataaaaagcactgtttttatcaaaacagtgataaaaatgaagaaaacctgcattgctctcaacaagagcattttgttttaagtgcgctcaaaacaactgaaataaatagactgccatcagcaaatcaaggctcaccatttaaatctgcgctctccactgtatctttttacaaccaaaataagtggt

SEQ ID NO: 16 - DNA, 2256 bp, Homo sapiens
gggcgcaggaattcaataaagaaaatggcagctcttactccaaggaagaggaagcaggattctttgaagtgtgacagccttttacacttcactgaaaatctgtttccatcacctaataaaaagcactgtttttatcaaaacagtgataaaaatgaagaaaacctgcattgctctcaacaagagcattttgttttaagtgcgctcaaaacaactgaaataaatagactgccatcagcaaatcaaggctcaccatttaaatctgcgctctccactgtatctttttacaaccaaaataagtggtacctcaatccactggagagaaagctgataaaagagagtagatctacttgtctaaaaactaatgatgaagataaatcttttcccattgtgacagaaaaaatgcaaggaaaaccagtctgctccaagaagaacaacaaaaaaccacagaagagtttaactgctaagtatcaaccaaagtatagacacatcaagcctgtatcaaggaattctagaaattccaagcaaaatcgagtgatctataagccaattgtggagaaggaaaataattgtcattcagctgaaaataattccaatgctcctcgggttctgagccaaaaaataaaaccacaagttacactccagggtggagcagcattttttgttagaaaaaaatcttctcttagaaaatcgtccctggaaaatgagccgtcactgggacgcacccaaaagagtaaatcagaagtcattgaagattctgatgtagagactgtcagtgaaaaaaaaacttttgcgacaaggcaagtgccaaagtgcttggtcctagaagagaaattgaaaattggactactgagtgcaagcagtaaaaataaagagaaattaataaaggtaaagctaaatatatcactttaaaaatggctgtataacaaaacttcagtataaatgacatagttgaataaaattttattttctggactgcttttataaagccagatagcatagtgtttagttactgtaaggagtttatttatttatttattttaagacagggtctttctttatgacccaggctgtagtgcagtggcacactgctcactacagcctcaacctcctaggctcaagcaatcccacctcagcctcccaagtagctggtactatagatgcacaccacctcacccggctaattcttgcattttttgtagaggctggggtttcaccatgttgcccaggctggtctcgcactgctgggctatagtgatccacctgcctcaccctcccaaattgctgggattatgggtgtgaaccactgcacctggcccagccacattttaagttctcaatagttacatgtagctggtggctaccatattagaccatgctggtctagaacttctgttgctgacctcctgtttatatccttcatcagaaaaaaagttgagttttgtggggcagctaggggcagaaagccattgaagcaaccttaagagaggttgtctccagatcagggcttccatgtatggtggcttgtttatattcatcacaacccttgtagctggagaaatgataggcttatatgatacccagtacactcaggcttatagtcatgtctttcctaacccacctctcatcattgccttcctcactagtttctctcaaattcaaccccagtccatatctctcgtaatcacctaaatttcaaacctcagtggcaaagataataaattggtggccagtaaaccaaatctcacccataaatgttttgcttggcctacataatttaaaagttggagccaaaattatttttaaatacaagaaaaacatttctggtgtctcatgaaatattgaaagctgagccttcatttctacatggcaataatcagagctgaatatcaggctgagtatcataccatttgacaaatatgggttcttcatttctctaagtcagtgtatatctatctatttagatcagtttagttttgatcattttacttactgctgcccttgtaaggcatttaagtttttagtcctcactgtaaatgtttgttttttttcctgtttatcgagtatggccaagtagattgttaaatgagatacgttcttgctagctaagccatgacagtctaattttactaattcattttctgtttatatcaacaaaacacttctgtgtcttgatagggattttgattataagagtaagctttatcaaaatgtattaaactgtgctttatacttaaaaaaaaaaaaaaaaaaaaaaaa

SEQ ID NO: 17 - DNA, 2170 bp, Homo sapiens
agatttttacctcaccagggcgcgagaatcttggaaacagccaccagggtgggcggggcaagggctggtgctccaccaatcaccgagccagcccctggaagcgctgagccgtggccaatcggaacgcagcggcttcctcctagcctggcgcgcgattatttgaagacgctcacggagcggctggctaggctgaggagagctcgccgggctctgaggcgcaggtaacctctggagtaggctgaggcggggggctgtggaaggctgaggcgagcgggagaccctgtggaacgtgggggcgaatgtcacggggaaagagcttttgctcggcgctcggcaggtccgcaggcccgagaccgaagaagccactgactcgttctcggcgctcacaggcgccgggtttccctcttcccgacgccggcgtttccttgctttgggtttagggtttgtttgtttgtttgtttccctgaccgggttcagggtcgggacgctttcctctcctacctttcctcgcttagccccagaggggtccccccttaagccgcagctcgacctgaattcccagagaccctggcctttcagtttgttcattcattaatagctttgaaataacgcttgtgtgctgctttggggggaatttccctgatcccagggaaggggaaggaaatgaattctgacgagccacgtagccgcctgctcgtgcattctagcatttgttttcagcagccttacgacttcaagagatttttataaaaagggaagcaaacggcttgctccgggctcatcagctaggaaaatggcagaggactgtgcttcacccgttttcttgctgggctctgatgaaaagttacagacggtctctgccttcattgaaggctgttgagatggggaaacaagaaaatgtcttaaatggactattagggaaatacagggtgccatggaagcagaacaagcgcgtgtaaccctgggaggggtggaaggagagcaatgtcgaggaagacctcccagaaaatagggttttaacttggtgtgaaatagactaattaaagggggtataattttgataaagcgatttttaatattttgatgaatgtggttattgtcatttcttttaggaattcaataaagaaaatggcagctcttactccaaggaagaggaagcaggattctttgaagtgtgacaggtgaatctcagcctgtgaatagaaactcttagaaaaatccaccttcttgtctctcttgttctctcctattttctaaaattttcgttcttccactagcctactccttgtggctacagtgataacctgataacctatattctaattctagcttggaaatacattaagttttcataatgaattatttagtccttttcaagggaatcaaagctgttttttagtttttgtttttttcttttgacatagaagcagcacattgaagaaagctgttttctatagctaggaaatataccaaatcagctattacctttttcctttctgttcccctataatattttacagtgttttgcatacagtagatgcttgtctggaaacaattctagaaagactgtgtactcagatattaattactctacccaggaaaagccccagggggtcagttcattacagacagttattatagggggcttgtagacaatagagcaaaatatatgagctataatttatatatggccaacacatagaaatcgattttaaaacatacatgcatatatgtctatcatagcttctttgtatatagtatttgttttcatgaactctttgggaagatatttgaatgttttatttgataaagacagaatttgagaatcctactgttaaactaatagaaaattgtgtgtatggggggtacgtgtattgtttttaatatgacctacaagtagagcttacttagcagtagatttatgtaaattttgacgcaaaataatcttatcaatggactttgtttctttttatagccttttacacttcactgaaaatctgtttccatcacctaataaaaagcactgtttttatcaaaacagtgataaaaatgaagaaaacctgcattgctctcaacaagagcattttgttttaagtgcgctcaaaacaactgaaataaatagactgccatcagcaaatcaaggctcaccatttaaatctgcgctctccac

SEQ ID NO: 18 - DNA, 487 bp, Homo sapiens
agatttttcacctcaccagggcgcgagaatcttggaaacagccaccagggtgggcggggcaagggctggtgctccaccaatcaccgagccagcccctggaagcgctgagccgtggccaatcggaacgcagcggcttcctcctagcctggcgcgcgattatttgaagacgctcacggagcggctggctaggctgaggagagctcgccgggctctgaggcgcaggaattcaataaagaaaatggcagctcttactccaaggaagaggaagcaggattctttgaagtgtgacagccttttacacttcactgaaaatctgtttccatcacctaataaaaagcactgtttttatcaaaacagtgataaaaatgaagaaaacccgcattgctctcaacaagagcattttgttttaagtgcgctcaaaacaactgaaataaatagactgccatcagcaaatcaaggctcaccatttaaatctgcgctctccact

SEQ ID NO: 19 - DNA, 3375 bp, Homo sapiens
atttgaagacgctcacggagcggctggctaggctgaggagagctcgccgggctctgaggcgcaggaattcaataaagaaaatggcagctcttactccaaggaagaggaagcaggattctttgaagtgtgacagccttttacacttcactgaaaatctgtttccatcacctaataaaaagcactgtttttatcaaaacagtgataaaaatgaagaaaacctgcattgctctcaacaagagcattttgttttaagtgcgctcaaaacaactgaaataaatagactgccatcagcaaatcaaggctcaccatttaaatctgcgctctccactgtatctttttacaaccaaaataagtggtacctcaatccactggagagaaagctgataaaagagagtagatctacttgtctaaaaactaatgatgaagataaatcttttcccattgtgacagaaaaaatgcaaggaaaaccagtctgctccaagaagaacaacaaaaaaccacagaagagtttaactgctaagtatcaaccaaagtatagacacatcaagcctgtatcaaggaattctagaaattccaagcaaaatcgagtgatctataagccaattgtggagaaggaaaataattgtcattcagctgaaaataattccaatgctcctcgggttctgagccaaaaaataaaaccacaagttacactccagggtggagcagcattttttgttagaaaaaaatcttctcttagaaaatcgtccctggaaaatgagccgtcactgggacgcacccaaaagagtaaatcagaagtcattgaagattctgatgtagagactgtcagtgaaaaaaaaacttttgcgacaaggcaagtgccaaagtgcttggtcctagaagagaaattgaaaattggactactgagtgcaagcagtaaaaataaagagaaattaataaaggattcatcagatgacagagtttcttcaaaggaacataaagttgataaaaatgaggctttttcttcagaggattctcttggtgagaataagacaatttctcctaagtccactgtctatccaatcttcagtgcatcttcagtcaattcaaaaagatctttaggtgaagaacagttttctgtgggatctgtcaacttcatgaaacagaccaatatccagaaaaatactaataccagagatacaagtaaaaaaacaaaagaccagctcatcatcgacgctggtcagaaacattttggggctactgtgtgcaagtcttgtggtatgatatatactgcttccaaccctgaagatgaaatgcagcatgtacagcatcaccacaggtttctggaaggaatcaaatatgtgggttggaagaaagaacgtgtagtagcagagttttgggatgggaaaatcgtgttggttctgccacatgatccaagctttgctatcaaaaaggtagaagatgtccaagaacttgttgataatgaattgggcttccagcaagttgttcctaaatgtccaaacaaaataaaaacttttctttttatatctgatgaaaagagagtagttgggtgtttaattgcagaacccatcaaacaggcatttcgtgtcctgtctgaaccaattggtccagaatccccaagctctacggaatgtcctagggcttggcaatgttcagatgtaccagaacctgcagtctgtgggataagtagaatctgggttttcagactgaagagaagaaagcgcattgcaagacgactggttgataccctcaggaattgcttcatgtttggctgttttctcagcactgatgaaatagcattttctgacccaacaccagatggcaagttatttgcaaccaagtactgcaacacccctaatttcctcgtatataattttaatagttaaagctgatttcagttataaaggagttactatctggataagttcaaagagctccttattataaaatacaaactatttaatatcaaaataaaaaataccgagactcacactcatacacacacacacacacacacgcacacacacatatcacagttttgttccttatgagttgaaaagtcaggaataaatttgttgaaaattatctggggattcaaaggaaaaatctttgggtgattccctgattagcactctgaatgtttaattatgaaactttgtagctataactggaaaattacctgactctttgtaagagtattaaatacaaagtgatttttctctagaaatgtgacctggtcttttataaagcccactcttagaccaggattatctaatgccacatcagaagcaaacaggcaaatttaaacttgggcaagtaatttctgtgcccaatttgtaaagggaattcctgaatttttttttttttttaatagaggcatgggtctcactgtgttgcccaggctggtctgaaacttttgggctcaagcgatcctcccaaaacgctgggattacagtcatgagccaccgtgcccagcctaattcctgacttctctatacagagtcttcacttgataggcactcgtctgtagtaactcagtttgaatatctttagaaaatgtttagaatttatttgtaacaagatggtaaggaataagattatcccatatgcatttctgtagagcagaatttgatagcttagtgttcaatctttttgaaaataaatgtttacctgtcatcagatttaattaaaattatacttagtaattgcactattacttagttaatttttgttgtatggaaatattggtagtactactttgggaacctgttactgacaattgatgtcattaacaaaatgcctagttggattagatgttttcattttctaattttttgcttgtttaaaatgcaccttacttgttctgagatacctggcaaaagtctttacaaaatgtatggtaatagaaccaaggttagtaaatatacataggctggtggatgagagaccatggaactgtgtaaatacacttaaatgttcacacatttttctagtgtaattcttggatactttaaaaagcaaaacattgttcaaattgttttgattctgaaaaatcattcaactgctaactggcaataagactctaggcaagtcgttttccagattgtaattatatgtagaaactattcatctgcattcattttatttgcctgtaagttaacatgtttccaaaatttaaaagcctgggtccccaaaagaatgtggaagtattaaaatgtatgtaattatgcaaacattttaatgctattttctgcacttatttcttttaaatattttatttaaaatttttaattaacattttgtttgcttaatgcttttgttatgaatcaattaaaattctttattttatacaactaaaaaaaaaaaaaaaaaaaa

SEQ ID NO: 20 - DNA, 1742 bp, Homo sapiens
cgttcttgctagctaagccatgacagtctaattttactaattcattttctgtttatatcaacaaaacacttctgtgtcttgatagggattttgattataagagtaagctttatcaaaatgtattaaactgtgctttatacttatacatttgtatgaaaattaagttttttgaaagtttggggcgtaactcaagctaaaattgtctcaatccagtaaaacaaaggaagagtctgcagtttccccatgcatctcctgatgattattcttatctgtttggtacagcctggtggtgtaccaaatgaatggtgacctgatgaattctctgtaaccagtgtaacagagaattctgtaattctcttttctgtcttattttctttttcttagcttcactttacgggtttggtattgagttagttttcagtagtttgggaagctgggcttttgtgcattttaaatttgtggaacggggctatgctctttcaagattttgctaaatacccatgtctgggattcgtctctgctagctaagggcgaatctgattttctgtttttttattttgagatggagtctcactccgttgcccaggctggagtgcagtggcatgatctcaactcactgcaacctccgcctcccaggttcaagcaattctctgcctcagcctcccaagtagctggtattgtaggcaccggccaccacacctggctaatttttgtatttttagtaggaacggggtttcaccctctcagccaggctggtcttgaactcctgactttgtgatccacctgccttggccacccaaagtgctggtattacaagtgtgagccaccgcgcctggctggtgaatctgatttttgtgaggataccctttccccaactcatggagccctgaaaagtgttaaactgatgggttatcccacctctgctgggatattatggttttgagtagaagttctactttaaccaagagtttttttcctttttccccaagctatatattccctagtaattcccaaattagtatgagggaaatctgtcaggatttttttctactttgcttttcccacattcctgctactctccagtgactctagaatgttctttccttagctgctgatttttaaagtttatggcaaaaagttgtccctttccagtttggtggctgctggggaaacttttgccatttttactggcttttttgttcatttcattatggaagtgatggtctgtaatcattctgccatctctgtcaggaagtataacacatttttgagaggatgacagattacaacacgtgtactctggcttttttttcttttgagacagtcttgctctgttgccaggctggagtgcagtggcacgatcttggctcactgcaacctccgcctcctgggttcaagcaattctcctgcctcagcctcccgagtagctgggactgcaggtgccaccacgcccagctaatttttgtatttttagtagagatagggtttcaccatgttagccaggatggtctcaatcccttgacctcgcaatctacccacctcggcctcccaaagtgctggcattacaggcatgagccaccgggccctgctgtactctggctttaaaggtaatgtacgcttagtgaatacaggggaagttttcctaaaaatagtttgaaaattgattgaaaatcacattatgaaatgtaattcataataaaccaaatcttttgtttttgaaaaaaaaaaaaaaaaaaaaaaaa

SEQ ID NO: 21 - DNA, 1068 bp, Homo sapiens
acgggtcgcgagaggttgttcgcgccttgagagttaagcgaagtgtggtggcttccaaggaatacaaacataaaggccttcgaccgttgcaaatagactaaagtgaaaacaaatctgaatgaagatgaagttatttcagaccatttgcaggcagctcaggagttcaaagttttctgtggaatcagctgcccttgtggctttctctacttcctcttactcatgtggccggaagaaaaaaagtgaacccatatgaagaagtggaccaagaaaaatactctaatttagttcagtctgtcttgtcatccagaggcgtcgcccagaccccgggatcggtggaggaagatgctttgctctgtggacccgtgagcaagcataagctgccaaaccaaggtgaggacagacgagtgccacaaaactggtttcctatcttcaatccagagagaagtgataaaccaaatgcaagtgatccttcagttcctttgaaaatccccttgcaaaggaatgtgataccaagtgtgacccgagtccttcagcagaccatggcaaaacaacaggttttcttgttggagaggtggaaacagcggatgattctggaactgggagaagatggctttaaagaatacacttcaaacgtctttttacaagggaaacggttccacgaagccttggaaagcatactttcaccccaggaaaccttaaaagagagagatgaaaatctcctcaagtctggttacattgaaagtgtccagcatattctgaaagatgtcagtggagtgcgagctcttgaaagtgctgttcaacatgaaaccttaaactatataggtctgctggactgtgtggctgagtatcagggcaagctctgtgtgattgattggaagacatcagagaaaccaaagccttttattcaaagtatatttgacaacccactgcaagttgtggcatacatgggtgccatgaaccatgataccaactacagctttcaggttcaatgtggcttaattgtggtggcctacaaagatggatcacctgcccacccacatttcatggatgcagagctctgttcccagt

SEQ ID NO: 22 - DNA, 2186 bp, Homo sapiens
acgggtcgcgagaggttgttcgcgccttgagagttaagcgaagtgtggtggcttccaaggaatacaaacataaaggccttcgaccgttgcaaatagactaaagtgaaaacaaatctgaatgaagatgaagttatttcagaccatttgcaggcagctcaggagttcaaagttttctgtggaatcagctgcccttgtggctttctctacttcctcttactcatgtggccggaagaaaaaaagtgaacccatatgaagaagtggaccaagaaaaatactctaatttagttcagtctgtcttgtcatccagaggcgtcgcccagaccccgggatcggtggaggaagatgctttgctctgtggacccgtgagcaagcataagctgccaaaccaaggtgaggacagacgagtgccacaaaactggtttcctatcttcaatccagagagaagtgataaaccaaatgcaagtgatccttcagttcctttgaaaatccccttgcaaaggaatgtgataccaagtgtgacccgagtccttcagcagaccatgacaaaacaacaggttttcttgttggagaggtggaaacagcggatgattctggaactgggagaagatggctttaaagaatacacttcaaacgtctttttacaagggaaacggttccacgaagccttggaaagcatactttcaccccaggaaaccttaaaagagagagatgaaaatctcctcaagtctggttacattgaaagtgtccagcatattctgaaagatgtcagtggagtgcgagctcttgaaagtgctgttcaacatgaaaccttaaactatataggtctgctggactgtgtggctgagtatcagggcaagctctgtgtgattgattggaagacatcagagaaaccaaagccttttattcaaagtacatttgacaacccactgcaagttgtggcatacatgggtgccatgaaccatgataccaactacagctttcaggttcaatgtggcttaattgtggtggcctacaaagatggatcacctgcccacccacatttcatggatgcagagctctgttcccagtactggaccaagtggcttcttcgactagaagaatatacggaaaagaaaaagaaccagaatattcagaaaccagaatattcagaatagggagcaagttgctatttgggaacattcagcaccttctcacagtttgggaacatatattgctgtttactccagtgtaaaaatgaggtgccactggatctgagtgctacacgaacacaagtagaagtattaatttgttgaaatgtgttgttaccaaaaagactgaaaagccccaaagtctagatataaagacctagacttcggcacgcgaaatcccagctatgctacctcttatttacctgaaaggaggacacgcaggatgggcagtcatgctggtgactcttgtactcccttgagggacattggtgggggggggggggcgtggtcccaggcaggatgcccagtctttgagctgagattggaaggcagtgaggctgagggtgccaagatttccccagggttcacccagaggggaaggggctacatgcccccagctgtgtgcagggaggacacatcagcccactaccgctgccaacaccaatgcctaaaacttgtttcatacattggggttttctatatatttcagttgggaaaagcttacatttaaccttttgaaaaaataaatacgtgattagcctcaactaaacattgctgactataaagacagtatattcaccatgtcgctggcaatatgtcattgcgtaacaccaaataaccccccagaagtagccagaggccagtttgaacatcacaattctaagtgttttagtaactatttctggcgtgagtcaacagatcatgtagatagagtcaattattgtttgtggagtttttcagctataggggaggggaactattaaaatccatttgtttctattcaataggtaataaaaattagttgtccctgggtttgggaaacttaaatgcccattacagccctggggaagggttttctgtcttatggagtgagtcttagcatttaagttatacagttgctgccttaaaatagtagcctgctacaatgacttctttgggtagccattttcataagaaataaaatacaagatatgagtaaaaaaaaaaaaaaaaaaaaaa

SEQ ID NO: 23 - DNA, 409 bp, Homo sapiens
ggcctaattcgaaaccaaagcgcgggacggatgaaagtacgggtcgcgagaggttgttcgcgccttgagagttaagcgaagtgtggtggcttccaaggaatacaaacataaaggccttcgaccgttgcaaatagactaaagtgaaaacaaatctgaatgaagatgaagttatttcagaccatttgcaggcagctcaggagttcaaagttttctgtggaatcagctgcccttgtggctttctctacttcctcttactcatgtggccggaagaaaaaagtgaacccatatgaagaagtggaccaagaaaaatactctaatttagttcagtctgtcttgtcatccagaggcgtcgcccagaccccgggatcggtggaggaagatgctttgctctgtggacccgtgagcaagc

SEQ ID NO: 24 - DNA, 1159 bp, Homo sapiens
acagcatgaagagttcattcttctgagtcaaggagaggtggaaaagctaatcaagtgcgacgaaattcaggtggattctgaagagccagtctttgaggctgtcatcaactgggtgaagcatgccaagaaagagcgggaagaatccttgcctaacctgctacagtatgtgcggatgcccctactaacccccaggtatatcacagatgtaatagatgctgagcctttcatccgctgtagtttacaatgcagggatctggttgatgaagcaaagaagtttcatctgaggcctgaacttcggagtcagatgcagggacccaggacaagggctcgcctaggagccaatgaagtgcttttggtggttgggggctttggaagccagcagtctcccattgatgtggtagagaaatatgaccccaagactcaggagtggagctttttgccaagcatcactcgtaagagacgttatgtggcctcagtgtcccttcatgaccggatctacgtcattggtggctatgatggccgttcccgccttagttcagtggaatgtctagactacacagcagatgaggatggggtctggtattctgtggcccctatgaatgtccgacgaggtcttgctggagccaccaccctgggagatatgatctatgtctctggaggctttgatggaagcaggcgtcacaccagtatggagcgctatgatccaaacattgaccagtggagcatgctgggagatatgcagacagcccgggaaggtgccggactcgtagtggccagtggagtgatctactgtctaggaggatatgacggcttgaatatcttaaattcagttgagaaatacgaccctcatacaggacattggactaatgttacaccaatggccaccaagcgttctgatatgatggtaattccctgctaagtagcattgaatgttatgaccctatcatcgacagctgggaagtcgtgacatccatgggaacccagcgctgtgatgctggtgtttgtgctctccgcgagaagtgaccattgttggagcaccatccagagctagtgaccagtccagtggacagttagtgggagaatcaaaaatcctttccagaatgtctgtttctcactacgtgcaccgggtgattacaggcaccagtgcagtgatgattgt

SEQ ID NO: 25 - DNA, 3561 bp, Homo sapiens
cacacctggccgtttattgagttttaagagttgtttatatatgtatttatttaatactagtcctttgtcagataggtggtttgcaaatattttttaattttgatgaagtccagcttatcaatttttctttttatggctcatgcttttggggtcaagtctaagaattccttgcctagccctagctagatcctcaagattttcttctggtcatcacgtcgcgtggccgcagggagcagacccggacagctccagagcctccgggccggggcggcggcggcgacgcttcggctcctcctgagccacctgctggacccgcaccccactccatccccacaggctggggacaggccctggcgcggctgtgtgggatcagaagcagagttgcagaatccaaggacctatttttgttctttctccgcactgctttatgggaggcattatggcccccaaagacataatgacaaatactcatgctaaatcaatcctcagttcaatgaactccgttgggaagagcaataccttctgtgatgtgacattgagtagagcagaaagactttcctgcccatgagattgtgctggctgcctgtagtgattacttctgtgccatgttcactagtgacctttatagaaggggaaaccctatgttgacatccaaggtttgactgcctctaccatggaaattttattggactttgtgtacacggaaacagtacatgtgacatggagaatgtacaggaactgcttcctgcagcctgtctgcttcagttgaaaggtgtgaaacaagcctgctgtgagttcttagaaagtcagttggacccttctaattgcctgggtattagggattttgctgaaacccacaattgtgttgacctgatgcaagcagctgaggtttttagccagaagcattttcctgaagtggtacagcatgaagagttcattcttctgagtcaaggagaggtggaaaagctaatcaagtgcgacgaaattcaggtggattctgaagagccagtctttgaggctgtcatcaactgggtgaagcatgccaagaaagagcgggaagaatccttgcctaacctgctacagtatgtgcggatgcccctactaacccccaggtatatcacagatgtaatagatgctgagcctttcatccgctgtagtttacaatgcagggatctggttgatgaagcaaagaagtttcatctgaggcctgaacttcggagtcagatgcagggacccaggacaagggctcgcctaggagccaatgaagtgcttttggtggttgggggctttggaagccagcagtctcccattgatgtggtagagaaatatgaccccaagactcaggagtggagctttttgccaagcatcactcgtaagagacgttatgtggcctcaatgtcccttcatgaccggatctacgtcattggtggctatgatggccgttcccgccttagttcagtggaatgtctagactacacagcagatgaggatggggtctggtattctgtggcccctatgaatgtccgacgaggtcttgctggagccaccaccctgggagatatgatctatgtctctggaggctttgatggaagcaggcgtcacaccagtatggagcgctatgatccaaacattgaccagtggagcatgctgggagatatgcagacagcccgggaaggtgccggactcgtagtggccagtggagtgatctactgtctaggaggatatgacggcttgaatatcttaaattcagttgagaaatacgaccctcatacaggacattggactaatgttacaccaatggccaccaagcgttctggtgcaggagtagccctgctgaatgaccatatttatgtggtggggggatttgatggtacagcccacctttcttccgttgaagcatacaacattcgcactgattcctggacaactgtcaccagtatgaccactccacgatgctatgtaggggccacagtgcttcgggggagactctatgcaattgcaggatatgatggtaattccctgctaagtagcattgaatgttatgaccctatcatcgacagctgggaagtcgtgacatccatgggaacccagcgctgtgatgctggtgtttgtgttctccgcgagaagtgaccgttgttggagcaccatccagagctagtgaccagtccagtggacagttagtgggagaatcaaaaatcctttccagaatgtctgtttctcactatgtgcaccgggtgattacaggcaccagtgcagtgatgattgtacttatttgacacatactccccgtcgtcctggttcttgttcctgagaagggtgggtaacagatattccaggaaaaagaatgcacattgaatggatgtgagagaccacattgcctctcccactgctttggggagcactttcctgtcatttctaacttaccacatgcttggtgtactatatgtatgttgtgcctcatatgttgcaaagaactaaggtgagtatagcctactagatatgggcaatatccagcctagatgattggaaagataccagtttaagtaaacttggtaaaatccaagtcttttttttttttttccaggaacaactacattttctcatatacaggtagctaggggcaacacagttccattctagagggaaacaaaagggagagccccacaaaactttggggacaagggagagagagactcatctgacacttcttttggaggtcaggatttgtatatcagaattgaagttagaattaagtgaattaaactgaatttgattgtgagtgaacctagaacagcactgaagtattacataacctggaagactgagaagggtatattatttgaaggatctttttatttccccgaggtctttcgcactggagacagcataaaagagtgaacaaatgttgggatgagagaagatgacatcaatgtgggagttcagtataactggggataaactagaagaacctgtgattttacagtcatcttattacctgccagggctcatctagccatggcaatgtttgccttgaatgggggtgaaagcctttctttgttggaccaaatactactacactattacacttccacactatttatttggggatgggctgggagtgacagtagcctagtagttcagctacctgattactgccccattcttttagaagcacatgtctgccaaggagtggtttgtactgctgtgtttggtacatctagtcttttttctgctataagttttccttacctgtcctttagtgtagattttattcatcacaggacagaataatcaaggacaaccaaaatccttttgttagtttcagtacctcagctatcaacatttctgagctaccattcaatgttcctctgtgtcatggagtgaaattcttgttttgtgggtattaggagtgtgggaatgtgataacctaaacaacctttgctctgaaattccatttttccctctttccctgagttgtattgacctacagagttaatttcctttgtatttttttaagaaaatattaaaaatcaacggtctcaaaaaaaaaaaaaaaaaaaa

SEQ ID NO: 26 - DNA, 1440 bp, Homo sapiens
aggggccagacccggacggctccagagcctccagagcctccgggtctgggcggcgcttcggctcctcccgagccgcctgctagccccgcgccgcactccatccccacaggctggggacgggccaggtgcggctgtgtgggttcgggagcggagttgcagaatccaaggacccattttgttctttctccgcactgctttatgggaggcattatggcccccaaagacataatgacaaatactcatgctaaatccatcctcaattcaatgaactccctcaggaagagcaataccctctgtgatgtgacattgagagtagagcagaaagacttccctgcccatcggattgtgctggctgcctgtagtgattacttctgtgccatgttcactagtgagctctcagagaaggggaaaccttatgttgacatccaaggtttgactgcctctaccatggaaattttattggactttgtgtacacagaaacagtacatgtgacagtggagaatgtacaagaactgcttcctgcagcctgtctgcttcagttgaaaggtgtgaaacaagcctgctgtgagttcttagaaagtcagttggacccttctaattgcctgggtattagggattttgctgaaacccacaattgtgttgacctgatgcaagcagctgaggtttttagccagaagcattttcctgaagtggtacagcatgaagagttcattcttctgagtcaaggagaggtggaaaagctaatcaagtgcgacgaaattcaggtggattctgaagagccagtctttgaggctgtcatcaactgggtgaagcatgccaagaaagagcgggaagaatccttgcctaacctgctacagtatgtgcggatgcccctactaacccccaggtatatcacagatgtaatagatgctgagcctttcatccgctgtagtttacaatgcagggatctggttgatgaagcaaagaagtttcatctgaggcctgaacttcggagtcagatgcagggacccaggacaagggctcgcctagatatgatctatgtctctggaggctttgatggaagcaggcgtcacaccagtatggagcgctatgatccaaacattgaccagtggagcatgctgggagatatgcagacagcccgggaaggtgccggactcgtagtggccagtggagtgatctactgtctaggaggatatgacggcttgaatatcttaaattcagttgagaaatacgaccctcatacaggacattggactaatgttacaccaatggccaccaagcgttctggtgcaggagtagccctgctgaatgaccatatttatgtggtggggggatttgatggtacagcccacctttcttccgttgaagcatacaacattcgcactgattcctggacaactgtcaccagtatgaccactccacgatgctatgtaggggcc

SEQ ID NO: 27 - DNA, 1126 bp, Homo sapiens
acctagagcccagctgaacaacaaggctttgggtgtgaagggactccccagcctggagaccctatttggctgaaacagttacaaaatatcaaatgtgttgtcagatattcctccaattgttcacatagctgggatatttgttgctcccctcaccccttggattatgtagggagccagtgcacacagcctgtttgttttagtatccaaggaagagaccaaggagccagctggcgggaaggggtgggggtgtgcagtctgccctgtccttctgctcataacctgacaaaatgccaaactagtaagcaggatagctgataccacggctatgagggagtaggctctgagagggcacagacttgtggagctgggcgtctggatcaaaactgctttgggatggaacctcgagccctagcagtgaagaagactccatttcttgtccaggggatttaaaagagttttctgctttgagagagaaatagagagtttagaaagcaattgctcttgggaaagctatacacagctctgttttgtcaatgacctttgttgtaagtctcccaacgtcccattaggagccacagcaggtgaggcatttggtgcagcaggaaacatggggactgcctaggctcgaatctgtggcaccctgagcaattacttaaattgtggagcctagttcctcatctgtaagatggacttgagattcctacctctcatgattactatggagattgaataattggtaaaattctcctagctcagtgactgccacaggatgggtctttcagattttggttctctttagcttctggttcttgaaagaaattaatctgtatataacataagaaactttgaaagtcaaaaaaacaaaaaattttaattcctcgtagattaattgatttgctatcttttagtttttttttctatgcatgtagatgtattaaatatgtaaatgtttttcaaagttgaggtaatattgtatagaattttatagcctacattttaatttcttgatatcttaagcatttcacttattagatattgttcaaaaatgccatttttaatatttgtataataccctatcatgtgcgtataccttaactaagccattcccatattcaacattttgtgt

SEQ ID NO: 28 - DNA, 3215 bp, Homo sapiens
gtgacttcctttttctgcccactctggtaacttattgctctgctgggctctttcccttagggtctctggccctgttcttgccccagcatgacttttatcgggacgccgttgtggaagcctcacgcaggagccctgcccccgtggagaagatcccactggtgactccaaccctaccaccatgaatggggtcctgatcccccatacgcccatcgcagtggacttctggagcctgcgccgggctggcaccgcacgtctcttcttcttgtctcacatgcactcggaccacaccgtgggcctgtctagcacctgggcccggcccctctactgctccccaattacagcccacctcttgcatcgtcacctacaggtgattttcgatacacaccatccatgctaaaggagccagccctgacactggggaaacagatccatactttatacctagacaacaccaattgcaatccagccctggttcttccttcccgacaagaagctgcccaccagattgtccagctcattcgaaaacacccacaacataacataaagattggactctacagcctgggaaaggaatcactgctggagcagctggccctggagtttcagacctgggtggtattgagtcctcggcgcctggagttggtacagctactgggcctggcagatgtgttcacagtggaggagaaggctggccgcatccatgcagtagaccatatggagatctgccattccaacatgctgcgttggaaccagacccaccctacgattgctatccttcccacaagccgaaaaatccacagctcccaccctgatatccacgtcatcccttactctgaccattcctcttactccgagcttcgtgcctttgtcgcagcactgaagccttgccaggtggtgcccattgtaagtcggcggccctgtggaggctttcaggacagtctgagccccaggatctccgtgcccctgattccggactctgtacagcaatacatgagttcttcctctagaaaaccaagccttctctggctgttagaaaggaggctaaagaggccgagaacccaaggtgttgtgtttgaatcccctgaggaaagtgctgatcaatctcaagctgacagagactcaaagaaggccaagaaagagaaactttctccctggcctgcggaccttgaaaagcagccttcccaccatcctttgcggatcaagaagcagttgttcccagatctctatagcaaagaatggaacaaggcagtgcctttctgcagggtattcttccaggagatttgaccagcaagtggaaaaataccataaaccctgctgaagacaggagagtacagaatgacaacattgagcccacactgcagttttgaagatagtaactgatggctggtgggaaagagtttgtttttggggcctacttttctatctttacaagactcttatgggcccaccgtggagcagcacttcccaaaacttgttcactggggtcctcgtgcctatggaatccttctttttataactaagtttaagaaatactttttttataaaatctttggagtatgcgtgagcaaattaaaagttctttgaagtcctacagtaacttaatctgtttaaccttgtttaacccagtatttctcaaacttttgtgaacatgcaatcatcttatgtgggtacagaaagaggtaaagagtctgaatcaaaaaggaccaggttattgctgttgctgttttgtggtgtcatgagccattctccatgtccccttctccctcttctcagatcaaaatccctagggagttctatttttaaaattatgaactatggcgctgcatgcttcaatcctgaacgtcactgacttgctgtgaccatccaaataattttcctgtctctgcctctgggagggaacaggaagcgatgaagaggtcttggaacagtagtgaaaattctacctctatgtccttcatgaggatgtgcagtatcccagtatcactgggatccatgtggaacagagccagctggggggttgggcagctctctccaaggcagtacctagagcccagctgaacaacaaggctttgggtgtgaagggactccccagcctggagaccctatttggctgaaacagttacaaaatatcaaatgtgttgtcagatattcctccaattgttcacatagctgggatatttgttgctcccctcaccccttggattatgtagggagccagtgcacacagcctgtttgttttagtatccaaggaagagaccaaggagccagctggcgggaaggggtgggggtgtgcagtctgccctgtccttctgctcataacctgacaaaatgccaaactagtaagcaggatagctgataccacggctatgagggagtaggctctgagagggcacagacttgtggagctgggcgtctggatcaaaactgctttgggatggaacctcgagccctagcagtgaagaagattccatttcttgtccaggggatttaaaagagttttctgctttgagagagaaatagagagtttagaaagcaattgctcttgggaaagctatacacagctctgttttgtcaatgacctttgttgtaagtctcccaacgtcctattaggagccacagcaggtgaggcatttggtgcagcaggaaacatggggactgcctaggctcgaatctgtggcaccctgagcaattacttaaattgtggagcctagttcctcatctgtaagatggacttgagattcctacctctcatgattactatggagattgaataattggtaaaattctcctagctcagtgactgccacaggatgggtctttcagattttggttctctttagcttctggttcttgaaagaaattaatctgtatataacataagaaactttgaaagtcaaaaaaacaaaaaattttaattcctcgtagattaattgatttgctatcttttagtttttttttctatgcatgtagatgtattaaatatgtaaatgtttttcaaagttgaggtaatattgtatagaattttatagcctacattttaatttcttgatatcttaagcatttcacttattagatattgttcaaaaatgccatttttaatatttgtataataccctatcatgtgagtataccttaactaagccattcccatattcaacattttgtgtactgtttttctaattacatatattacaatgaaaaaaaaaaaaaaaaaa

SEQ ID NO: 29 - DNA, 3183 bp, Homo sapiens
attttggaaccatcctctacacaggtgattttcgatacacaccatccatgctaaaggagccagccctgacactggggaaacagatccatactttatacctagacaacaccaattgcaatccagccctggttcttccttcccgacaagaagctgcccaccagattgtccagctcattcgaaaacacccacaacataacataaagattggactctacagcctgggaaaggaatcactgctggagcagctggccctggagtttcagacctgggtggtattgagtcctcggcgcctggagttggtacagctactgggcctggcagatgtgttcacagtggaggagaaggctggccgcatccatgcagtagaccatatggagatctgccattccaacatgctgcgttggaaccagacccaccctacgattgctatccttcccacaagccgaaaaatccacagctcccaccctgatatccacgtcatcccttactctgaccattcctcttactccgagcttcgtgcctttgtcgcagcactgaagccttgccaggtggtgcccattgtaagtcggcggccctgtggaggctttcaggacagtctgagccccaggatctccgtgcccctgattccggactctgtacagcaatacatgagttcttcctctagaaaaccaagccttctctggctgttagaaaggaggctaaagaggccgagaacccaaggtgttgtgtttgaatcccctgaggaaagtgctgatcaatctcaagctgacagagactcaaagaaggccaagaaagagaaactttctccctggcctgcggaccttgaaaagcagccttcccaccatcctttgcggatcaagaagcagttgttcccagatctctatagcaaagaatggaacaaggcagtgcctttctgtgagtctcaaaagagggtgactatgttgacggccccactgggattttcagtgcacttaaggtctacagatgaggagtttatttctcaaaaaaccagggaggaaattggtttagggtcccccttggtacccatgggagatgatgatggaggtccagaagccacagggaatcagagtgcctggatgggccatggttctcccctgtcccacagcagcaagggcacccctcttctagctactgaattcaggggtctagcactcaaatatcttctgactccagtgaactttttccaggcagggtattcttccaggagatttgaccagcaagtggaaaaataccataaaccctgctgaagacaggagagtacagaatgacaacattgagcccacactgcagttttgaagatagtaactgatggctggtgggaaagagtttgtttttggggcctacttttctatctttacaagactcttatgggcccaccgtggagcagcacttcccaaaacttgttcactggggtcctcgtgcctatggaatccttctttttataactaagtttaagaaatactttttttataaaatctttggagtatgcgtgagcaaattaaaagttctttgaagtcctacagtaacttaatctgtttaaccttgtttaacccagtatttctcaaacttttgtgaacatgcaatcatcttatgtgggtacagaaagaggtaaagagtctgaatcaaaaaggaccaggttattgctgttgctgttttgtggtgtcatgagccattctccatgtccccttctccctcttctcagatcaaaatccctagggagttctatttttaaaattatgaactatggcgctgcatgcttcaatcctgaacgtcactgacttgctgtgaccatccaaataattttcctgtctctgcctctgggagggaacaggaagcgatgaagaggtcttggaacagtagtgaaaattctacctctatgtccttcatgaggatgtgcagtatcccagtatcactgggatccatgtggaacagagccagctggggggttgggcagctctctccaaggcagtacctagagcccagctgaacaacaaggctttgggtgtgaagggactccccagcctggagaccctatttggctgaaacagttacaaaatatcaaatgtgttgtcagatattcctccaattgttcacatagctgggatatttgttgctcccctcaccccttggattatgtagggagccagtgcacacagcctgtttgttttagtatccaaggaagagaccaaggagccagctggcgggaaggggtgggggtgtgcagtctgccctgtacttctgctcataacctgacaaaatgccaaactagtaagcaggatagctgataccacggctatgagggagtaggctctgagagggcacagacttgtggagctgggcgtctggatcaaaactgctttgggatggaacctcgagccctagcagtgaagaagattccatttcttgtccaggggatttaaaagagttttctgctttgagagagaaatagagagtttagaaagcaattgctcttgggaaagctatacacagctctgttttgtcaatgacctttgttgtaagtctcccaacgtcctattaggagccacagcaggtgaggcatttggtgcagcaggaaacatggggactgcctaggctcgaatctgtggcaccctgagcaattacttaaattgtggagcctagttcctcatctgtaagatggacttgagattcctacctctcatgattactatggagattgaataattggtaaaattctcctagctcagtgactgccacaggatgggtctttcagattttggttctctttagcttctggttcttgaaagaaattaatctgtatataacataagaaactttgaaagtcaaaaaaacaaaaaattttaattcctcgtagattaattgatttgctatcttttagtttttttttctatgcatgtagatgtattaaatatgtaaatgtttttcaaagttgaggtaatattgtatagaattttatagcctacattttaatttcttgatatcttaagcatttcacttattagatattgttcaaaaatgccatttttaatatttgtataataccctatcatgtgagtataccttaactaagccattcccatattcaacattttgtgtactgtttttctaattacatatattacaatgaacaaccttatgcaaaaaaaaaaaaaaaaaaaaaaa

SEQ ID NO: 30 - DNA, 1157 bp, Homo sapiens
ggcggttgggagtgtccagcgccctccgcgatttgggctccagcgggcagggtgacttcctttttctgcccactctggtaacttattgctctgctgggctctttcccttagggtctctggccctgttcttgccccagcatgacttttatcgggacgccgttgtggaagcctcacgcaggagccctgcccccgtggagaagatcccactggtgactccaaccctaccaccatgaatggggtcctgatcccccatacgcccatcgcagtggacttctggagcctgcgccgggctggcaccgcacgtctcttcttcttgtctcacatgcactcggaccacaccgtgggcctgtctagcacctgggcccggcccctctactgctccccaattacagcccacctcttgcatcgtcacctacaggtatctaagcaatggatccaagccctggaggttggtgagagccatgtattacccctagatgaaattggacaagagaccatgaccgtaaccctcctcgatgccaatcactgtcctggttctgtcatgtttctctttgaaggatattttggaaccatcctctacacaggtgattttcgatacacaccatccatgctaaaggagccagccctgacactggggaaacagatccatactttatacctagacaacaccaattgcaatccagccctggttcttccttcccgacaagaagctgcccaccagattgtccagctcattcgaaaacacccacaacataacataaagattggactctacagcctgggaaaggaatcactgctggagcagctggccctggagtttcagacctgggtggtattgagtcctcggcgcctggagttggtacagctactgggcctggcagatgtgttcacagtggaggagaaggctggccgcatccatgcagtagaccatatggagatctgccattccaacatgctgcgttggaaccagacccaccctacgattgctatccttcccacaagccgaaaaatccacagctcccaccctgatatccacgtcatcccttactctgaccattcctcttactccgagcttcgtgcctttgtcgcagcactgaagccttgccaggtggtgcccattgtaagtcggcggccctgggaggctttcaggacagtct

SEQ ID NO: 31 - DNA, 1014 bp, Homo sapiens
ggaagatggcgacggccttgagcgaggaggagctggacaatgaagactattactcgttgctgaacgtgcgcagggaggcctcttctgaagagctgaaagctgcctaccggaggctctgtatgctctaccatccagacaagcacagagacccagagctcaagtcacaggcggaacgactgtttaaccttgttcaccaggcttatgaagtgcttagtgacccccaaaccagggccatctatgatatatatgggaagggaggactggaaatggaaggatgggaggttgtggaaaggaggagaacccctgctgaaattcgagaggagtttgagcggctgcagagagagagagaagagaggagattgcagcagcgaaccaatcccaagggaacgatcagcgttggagtaaatgccaccgacctttttgatcgctatgatgaggagtatgaagatgtgtccggcagtagctttccgcagattgaaattaataaaatgcacatatcccagtccattgaggcacccttgacagcgacagacacagccatcctctctggaagcctctcaacccagaatggaaatggaggaggttccattaactttgcgctcagacgagtaacttcggtaaagggatggggagagttggaatttggagctggagacctacaggggcctttgttcggtctcaagctgttccgtaatctcacaccaagatgctttgtgacaacaaactgtgctctgcagttttcatcccgtggaatccgacccggcctgaccactgtcctagctcggaacctagacaagaacaccgtgggctacctgcagtggcgatggggtatccagtcagccatgaacactagcatcgtccgagacactaaaaccagccacttcactgtggccctgcagctgggaatccctcactcctttgcactgatcagctatcagcacaaattccaagatgacgatcagactcgtgtgaagggatccctcaaagcaggcttctttgggacggtggtggagt

SEQ ID NO: 32 - DNA, 3230 bp, Homo sapiens
gggggtgaaaggttgcgaagatggcgacggccttgagcgaggaggagctggacaatgaagactattactcgttgctgaacgtgcgcagggaggcctcttctgaagagctgaaagctgcctaccggaggctctgtatgctctaccatccagacaagcacagagacccagagctcaagtcacaggcggaacgactgtttaaccttgttcaccaggcttatgaagtgcttagtgacccccaaaccagggccatctatgatatatatgggaagagaggactggaaatggaaggatgggaggttgtggaaaggaggagaacccctgctgaaattcgagaggagtttgagcggctgcagagagagagagaagagaggagattgcagcagcgaaccaatcccaagggaacgatcagcgttggagtagatgccaccgacctttttgatcgctatgatgaggagtatgaagatgtgtccggcagtagctttccgcagattgaaattaataaaatgcacatatcccagtccattgaggcacccttgacagcgacagacacagccatcctctctggaagcctctcaacccagaatggaaatggaggaggttccattaactttgcgctcagacgagtaacttcggcaaagggatggggagagttggaatttggagctggagacctacaggggcctttgttcggtctcaagctgttccgtaatctcacaccaagatgctttgtgacaacaaactgtgctctgcagttttcatcccgtggaatccgacccggcctgaccactgtcctagctcggaacctagacaagaacaccgtgggctacctgcagtggcgatggggtatccagtcagccatgaacactagcatcgtccgagacactaaaaccagccacttcactgtggccctgcagctgggaatccctcactcctttgcactgatcagctatcagcacaaattccaagatgacgatcagactcgtgtgaaaggatccctcaaagcaggcttctttgggacggtggtggagtacggagctgagaggaagatctccaggcacagcgttttgggtgcagctgtcagcgttggagttccacagggcgtttctctcaaagtcaagctcaacagggccagtcagacatacttcttccctattcacttgacggaccagcttctgcccagcgccatgttctatgccaccgtggggcctctagtggtctactttgccatgcaccgtctgatcatcaaaccatacctcagggctcagaaagagaaggaattggagaagcagagggaaagcgccgccaccgatgtgctgcagaagaagcaagaggcggagtccgctgtccggctgatgcaggaatctgtccgaaggataattgaggcagaagagtccagaatgggcctcatcatcgtcaatgcctggtacgggaagtttgtcaatgacaagagcaggaagagcgagaaggtgaaggtgattgacgtgactgtgcccctgcagtgcctggtgaaggactcgaagctcatcctcacggaggcctccaaggctgggctgcctggcttttatgacccgtgtgtgggggaagagaagaacctgaaagtgctctatcagttccggggcgtcctgcatcaggtgatggtgctggacagtgaggccctccggataccaaagcagtcccacaggatcgatacagatggataaactgccaagaaccagatttttaaaaggccgcaaaaaatcttttcctgggagtctacaaatttggaaatgaaaaaacccagacatcagatgtttttattttatattattattatagaaggtggtaccattatcaattatgtgaagggacatgcagacaccccagcttttgagggtgctgggggtaggactgaggcagccccactgggaaccagactgcagcctggcccatggctgttttcccaaggatcagttcctggagggaagggctctggccctgactccgctgtgtcccgagcacacgtgctgaccgcagcccgccgccctgtagttcttggctgggtctggaggtgtctgtggagcaccctgccctcaccacaggagcgtgagccacttctgcagtccacgctgaacatgggaaacaacctgaaaagcaggcaggcctcccggtcagggagcctctgctgtgctggcttcccatgaccacctcctcctgctgaaatattactgcttgaatctggagcagattgcgggtttataaaactgctttttatctgagaacaaacgggtttggaaattagtcgtcttttttccccactcccagagctgctcaaatcattccaccggccccctcggcttgggacagggtagtgtaactcccgatcccagggcctagccctgacacaggtggcttcccgtatcccggtgggaaaacgccctgccaccagcgggcttgagctggcctgtgtccctccactgcctgcaccacccacctccagagtgcagtgctgggcaagggcagctcaagaggacaggaccaggcgcttggcaagacatcagacacacccaacccaaaggcgtggaccccaggcccggcccgtggtacccagcaggtggcactgcagctccccgctcctgcaggtccagcgtcctcacaggaacaccagggcctgtgctccggagccttccttcagacccttcctccacgtgcccacttgggatgcagaatgcagcggagctaggaccccctccacggcctggacctcggctgcagtaaagttacgtgaggcctgtctctcggggcctggaagtggcagccatcagttgctcttgctgacccctcggagcaagcgccgcacaggtggtggctgagacagctggcgtggggggccccaagctgcgccggcctccagcccacccacagctgttgctgaagtcaggcctccctccccagcactggtatctgagtaacggctaagaacctccttcctctggttttgaaaagcagttcgggttgtccaattctgtaacattcatctccattttttaaaaaggtttctctgacggccccacggcccgagccacggtgagcgtcgtgttgcatgagcctgggccccgggcttcccgtgcgcctctgccgcaggtgcttctgggcacccatcctctgcgtttcatttgcagtcgactgtacagaaggcactcaccacaataaacctttcctgaaagcagaaaaaaaaaaaaaaaaaaaaaaaa

SEQ ID NO: 33 - DNA, 355 bp, Homo sapiens
gagctgagataatgcacggcacttcagcttgggcgacagagcgagactccgtctcaaaaaagaaaaaaaagaaagaaattcataaggttgtggaaaggaggagaacccctgctgaaattcgagaggagtttgagcggctgcagagagagagagaagagaggagattgcagcagcgaaccaatcccaagggaacgatcagcgttggagtagatgccaccgacctttttgatcgctatgatgaggagtatgaagatgtgtccggcagtagctttccgcagattgaaattaataaaatgcacatatcccagtccattgaggcacccttgacagcgacagacacagccatcctctctgg

SEQ ID NO: 34 - DNA, 454 bp, Homo sapiens
acaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgt

SEQ ID NO: 35 - DNA, 3661 bp, Homo sapiens
cgtcgctgcttcggtgtccctgtcgggcttcccagcagcggcctagcgggaaaagtaaaagatgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtagacagtggggttgtggttggttggtatagaatggtgggaattcaaatttttctaaactcatggtaagtatattgtaaaatacatatgtactaagaattttcaaaattggtttgttcagtgtggagtatattcagcagtatttttgacatttttctttagaaaaaggaagagctaaaggaattttataagttttgttacatgaaaggttgaaatattgagtggttgaaagtgaactgctgtttgcctgattggtaaaccaacacactacaattgatatcaaaaggtttctcctgtaatattttatccctggacttgtcaagtgaattctttgcatgttcaaaacggaaaccattgattagaactacattctttaccccttgttttaatttgaaccccaccatatggatttttttccttaagaaaatctccttttaggagatcatggtgtcacagtgtttggttcttttgttttgttttttaacacttgtctcccctcatacacaaaagtacaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgtacgaatgttttttgcattcaaaggacatccacatctgttggaagacttttaagtgagtttttgttcttagataacccacattagatgaatgtgttaagtgaaatgatacttgtactccccctacccctttgtcaactgctgtgaatgctgtatggtgtgtgttctcttctgttactgatatgtaagtgtggcaatgtgaactgaagctgatgggctgagaacatggactgagcttgtggtgtgctttgcaggaggacttgaagcagagttcaccagtgagctcaggtgtctcaaagaagggtggaagttctaatgtctgttagctacccataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattccccttttttgacccttttgagatggaactttcataaagtttcttggcagtagtttattttgcttcaaataaacttatttgaaaagttgtctcaagtcaaatggattcatcacctgtcatgcattgacacctgatacccagacttaattggtatttgttcttgcattggccaaagtgaaaattttttttttttcttttgaaatctagttttgaataagtctgggtgaccgcacctaaaatggtaagcagtaccctccggctttttcttagtgcctctgtgcatttgggtgatgttctatttacatggcctgtgtaaatctccattgggaagtcatgccttctaaaaagattcttatttgggggagtgggcaaaatgttgattattttctaatgctttgtagcaaagcatatcaattgaaaagggaatatcagcaccttcctagtttgggatttgaaaagtggaattaattgcagtagggataaagtagaagaaaccacaaattatcttgtgcctgaaatccattaagaggcctgatagctttaagaattagggtgggttgtctgtctggaagtgttaagtggaatgggctttgtcctccaggaggtgggggaatgtggtaacattgaatacagttgaataaaatcgcttacaaaactcacactctcacaatgcattgttaagtatgtaaaagcaataacattgattctctgttgtacttttttgtaactaattctgtgagagttgagctcattttctagttggaagaatgtgatatttgttgtgttggtagtttacctaatgcccttacctaattagattatgataaataggtttgtcattttgcaagttacatgttttaaacatttatcaatgaagtcatcctttagacttgtaaaaaaaaaaaaaaaagaaa

SEQ ID NO: 36 - DNA, 1095 bp, Homo sapiens
tcgagcggccgcccacgcgggcacaccaagaatcagctgaacctaaataccttcctcataaaacatgtaacgaaattattgtgcctaaagccccctctcataaaacaatccaagaaacacctcattctgaagactattcaattgaaataaaccaagaaactcctgggtctgaaaaatattcacctgaaacgtatcaagaaatacctgggcttgaagaatattcacctgaaatataccaagaaacatcccagcttgaagaatattcacctgaaatataccaagaaacaccggggcctgaagacctctctactgagacatataaaaataaggatgtgcctaaagaatgctttccagaaccacaccaagaaacaggtgggccccaaggccaggatcctaaagcacaccaggaagatgctaaagatgcttatacttttcctcaagaaatgaaagaaaaacccaaagaagagccaggaataccagcaattctgaatgagagtcatccagaaaatgatgtctatagttatgttttgttttaacaatgctcaaccataaagttgtggtccaatggaacatacagcttaatagtttatgcgtgattttctcaaaatattgtaaaacttttgacaatgctcattaatattattttttctatttgtagaccatatctgaaagaaataacattttttaaggctctaccacatagacaatatcatgctagaatgtgtgtgtgtgtgtgtgtgtgtatgtatgtataggtcggggagaggatagtggtgggaacagacaaataaggaagcggggaggactggataattggttttcccccctaagaacatttatttacgtcttaagagcagataagtgactaagactgaacacatacattttgtggagtatatagttttcttgtaaatgctgttcaattattaatgtaacagtagcatcaaaattttattcaggctttagttgactcttttggtcagttttaacaattctccttaaaagatattttggagtgatgaatgtggtttacttttgtatttgaattttgattttctatttttattttttaaatattgtatttgtgcacaatgt

SEQ ID NO: 37 - DNA, 2103 bp, Homo sapiens
ggaagtcagaccaaaatagcaggaaggtattgcagcaagatggatttgggaaaggaccaatctcatttgaagcaccatcagacacctgaccctcatcaagaagagaaccattctccagaagtcattggaacctggagtttgagaaacagagaactacttagaaaaagaaaagctgaagtgcatgaaaaggaaacatcacaatggctatttggagaacagaaaaaacgcaagcagcagagaacaggaaaaggaaatcgaagaggcagaaagagacaacaaaacacagaattgaaggtggagcctcagccacagatagaaaaggaaatagtggagaaagcactggcacctatagagaaaaaaactgagccacctgggagcataaccaaagtatttccttcagtagcctccccgcaaaaagttgtgcctgaggaacacttttctgaaatatgtcaagaaagtaacatatatcaggagaatttttctgagtaccaagaaatagcagtacaaaaccattcttctgaaacatgccaacatgtgtctgaacctgaagacctctctcctaaaatgtaccaagaaatatctgtacttcaagacaattcttccaaaatatgccaagacatgaaggaacctgaagacaactctcctaacacatgccaagtaatatctgtaattcaagaccatcctttcaaaatgtaccaagatatggctaaacgagaagatctggctcctaaaatgtgccaagaagctgctgtacccaaaatccttccttgtccaacatctgaagacacagctgatctggcaggatgctctcttcaagcatatccaaaaccagatgtgcctaaaggctatattcttgacacagaccaaaatccagcagaaccagaggaatacaatgaaacagatcaaggaatagctgagacagaaggcctttttcctaaaatacaagaaatagctgagcctaaagacctttctacaaaaacacaccaagaatcagctgaacctaaataccttcctcataaaacatgtaacgaaattattgtgcctaaagccccctctcataaaacaatccaagaaacacctcattctgaagactattcaattgaaataaaccaagaaactcctgggtctgaaaaatattcacctgaaacgtatcaagaaatacctgggcttgaagaatattcacctgaaatataccaagaaacatcccagcttgaagaatattcacctgaaatataccaagaaacaccggggcctgaagacctctctactgagacatataaaaataaggatgtgcctaaagaatgctttccagaaccacaccaagaaacaggtgggccccaaggccaggatcctaaagcacaccaggaagatgctaaagatgcttatacttttcctcaagaaatgaaagaaaaacccaaagaagagccaggaataccagcaattctgaatgagagtcatccagaaaatgatgtctatagttatgttttgttttaacaatgctcaaccataaagttgtggtccaatggaacatacagcttaatagtttatgcgtgattttctcaaaatattgtaaaacttttgacaatgctcattaatattattttttctatttgtagaccatatctgaaagaaataacattttttaaggctctaccacatagacaatatcatgctagaatgtgtgtgtgtgtgtgtgtgtgtgtgtgtgtatgtatgtataggtcggggagaggatagtggtgggaacagacaaataaggaagcggggaggactggataattggttttcccccctaagaacatttatttacgtcttaagagcagataagtgactaagactgaacacatacattttgtggagtatatagttttcttgtaaatgctgttcaattattaatgtaacagtagcatcaaaattttattcaggctttagttgactcttttggtcagttttaacaattctccttaaaagatattttggagtgatgaatgtagtttacttttgtatttgaattttgattttctatttttattttttaaatattgtatttgtgcacaatgtacattaaatcattattacatgcttaaaaaaaaaaaaaaaaaa

SEQ ID NO: 38 - DNA, 1516 bp, Homo sapiens
gcaagatggatttgggaaaggaccaatctcatttgaagcaccatcagacacctgaccctcatcaagaagagaaccattctccagaagtcattggaacctggagtttgagaaacagagaactacttagaaaaagaaaagctgaagtgcatgaaaaggaaacatcacaatggctatttggagaacagaaaaaacgcaagcagcagagaacaggaaaaggaaatcgaagaggcagaaagagacaacaaaacacagaattgaaggtggagcctcagccacagatagaaaaggaaatagtggagaaagcactggcacctatagagaaaaaaactgagccacctgggagcataaccaaagtatttccttcagtagcctccccgcaaaaagttgtgcctgaggaacacttttctgaaatatgtcaagaaagtaacatatatcaggagaatttttctgagtaccaagaaatagcagtacaaaaccattcttctgaaacatgccaacatgtgtctgaacctgaagacctctctcctaaaatgtaccaagaaatatctgtacttcaagacaattcttccaaaatatgccaagacatgaaggaacctgaagacaactctcctaacacatgccaagtaatatctgtaattcaagaccatcctttcaaaatgtaccaagatatggctaaacgagaagatctggctcctaaaatgtgccaagaagctgctgtacccaaaatccttccttgtccaacatctgaagacacagctgatctggcaggatgctctcttcaagcatatccaaaaccagatgtgcctaaaggctatattcttgacacagaccaaaatccagcagaaccagaggaatacaatgaaacagatcaaggaatagctgagacagaaggcctttttcctaaaatacaagaaatagctgagcctaaagacctttctacaaaaacacaccaagaatcagctgaacctaaataccttcctcataaaacatgtaacgaaattattgtgcctaaagccccctctcataaaacaatccaagaaacacctcattctgaagactattcaattgaaataaaccaagaaactcctgggtctgaaaaatattcacctgaaacgtatcaagaaatacctgggcttgaagaatattcacctgaaatataccaagaaacatcccagcttgaagaatattcacctgaaatataccaagaaacaccggggcctgaagacctctctactgagacatataaaaataaggatgtgcctaaagaatgctttccagaaccacaccaagaaacaggtgggccccaaggccaggatcctaaagcacaccaggaagatgctaaagatgcttatacttttcctcaagaaatgaaagaaaaacccaaagaagagccaggaataccagcaattctgaatgagagtcatccagaaaatgatgtctatagttatgttttgttttaacaatgctcaaccataaagttgtggtccaatggaacataaaaaaaaaaaaaaaaaaa

SEQ ID NO: 39 - DNA, 426 bp, Homo sapiens
acaactggcattcaaatctaggtcagtctgcccccagagccactacccttacccctcactgaatctgcctttatattgttgagcccatgaccccaaactgctctttccaatttgaacttccagggattttattgtgaacttacatagcaacattaaaatgaagttgaattgtttttaatggcaacgccgtctgtctcctctagcttaccgcttctcacctttcaaccccatctgtggcctttgtccaggcccacagcttagccatggcttccctcctgcatccctgccgtgggttgctggcctcacacttgcagcagctggacagtgattttagaaggccaccagtccccatagctatgtgacaatgagaagcaaacttttttgtgacagattgtattggcataggcatgatagatggggattggt

SEQ ID NO: 40 - DNA, 2864 bp, Homo sapiens
ctcgcttctcgttctactgccccaggagcccggcgggtccgggactcccgtccgtgccggtgcgggcgccggcatgtggctgtgggaggaccagggcggcctcctgggccctttctccttcctgctgctagtgctgctgctggtgacgcggagcccggtcaatgcctgcctcctcaccggcagcctcttcgttctactgcgcgtcttcagctttgagccggtgccctcttgcagggccctgcaggtgctcaagccccgggaccgcatttctgccatcgcccaccgtggcggcagccacgacgcgcccgagaacacgctggcggccattcggcaggcagctaagaatggagcaacaggcgtggagttggacattgagtttacttctgacgggattcctgtcttaatgcacgataacacagtagataggacgactgatgggaccgggcgattgtgtgatttgacatttgaacaaattaggaagctgaatcctgcagcaaaccacagactcaggaatgatttccctgatgaaaagatccctaccctaagggaagctgttgcagagtgcctaaaccataacctcacaatcttctttgatgtcaaaggccatgcacacaaggctactgaggctctaaagaaaatgtatatggaatttcctcaactgtataataatagtgtggtctgttctttcttgccagaagttatctacaagatgagacaaacagatcgggatgtaataacagcattaactcacagaccttggagcctaagccatacaggagatgggaaaccacgctatgatactttctggaaacattttatatttgttatgatggacattttgctcgattggagcatgcataatatcttgtggtacctgtgtggaatttcagctttcctcatgcaaaaggattttgtatccccggcctacttgaagaagtggtcagctaaaggaatccaggttgttggttggactgttaatacctttgatgaaaagagttactacgaatcccatcttggttccagctatatcactgacagcatggtagaagactgcgaacctcacttctagactttcacggtgggacgaaacgggttcagaaactgccaggggcctcatacagggatatcaaaataccctttgtgctagcccaagccctggggaatcaggtgactcacacaaatgcaatagttggtcactgcatttttacctgaaccaaagctaaacccggtgttgccaccatgcaccatggcatgccagagttcaacactgttgctcttgaaaatctgggtctgaaaaaacgcacaagagcccctgccctgccctagctgaggcacacagggagacccagtgaggataagcacagattgaattgtacaatttgcagatgcagatgtaaatgcatgggacatgcatgataactcagagttgacattttaaaacttgccacacttatttcaaatatttgtactcagctatgttaacatgtactgtagacatcaaacttgtggtcatactaataaaattattaaaaggagcactaaaggaaaactgtgtgccaagcatcatatcctaaggcatacggaatttggggaagccaccatgcaatccagtgaggcttcagtgtacagcaaccaaaatggtagggaggtcttgaagccaatgagggatttatagcatcttgaatagagagctgcaaaccaccagggggcagagttgcacttttccaggctttttaggaagctctgcaacagatgtgatctgatcataggcaattagaactggaagaaacttccaaaaatatctaggtttgtcctcattttacaaatgaggaaactaaactctgtggaagggaaggggttgcctcaaaagtcacagcttagctgggcacagtggctcatgccgataatcccagcaattcagaaagctgaggcaggaggattacttgaggccagactgggcaatatagcaagaccccatctctaaaaaattaggcatggtggtgcatgcctgtattcccagctactcaggaggttgaggtgggaggatcacttgagcccagaagttcaaggttgcaatgagccatgattacaccacggcactacaaccttggtggcacagtgagaacctgactcttaaaaaaaaaaaaaaaaaaaaaaaagataactagaacttctagaacatcttgtttacagttagccagaaactatacaagtggtttaacatgcattatcttactcaatccatacaaaagtcttatggaggtgttagcactctttctactgatgaagaactgaggtacttcataaaaccacttacccaaggtgtcttgagtctggtacaactggcattcaaatctaggtcagtctgcccccagagccactacccttacccctcactgaatctgcctttatattgttgagcccatgaccccaaactgctctttccaatttgaacttccagggattttattgtgaacttacatagcaacattaaaatgaagttgaattgtttttaatggcaacgccgtctgtctcctctagcttaccgcttctcacctttcaaccccatctgtggcctttgtccaggcccacagcttagccatggcttccctcctgcatccctgccgtgggttgctggcctcacacttgcagcagctggacagtgattttagaaggccaccagtccccatagctatgtgacaatgagaagcaaacttttttgtgacagattgtattggcataggcatgatagatggggattggtacgttttgaatcagcatttgcaaaaaaattgtcttgaattttaaaataaacaacaaagatttgttcattgagtgcaaaaaaaaaaaaaaaaaaaaaaaaa

SEQ ID NO: 41 - DNA, 704 bp, Homo sapiens
acttcacgactggctgccactactgggaggtgtatgtgggagacaagaccaaatggattcttggagtatgtagtgagtcagtgagcaggaaggggaaggttactgcctcacctgccaatggacactggcttctgcgacagagtcgtgggaatgagtatgaagctctcacatccccgcagacctccttccgccttaaagagcctccacggtgtgtggggattttcctggactatgaagcaggagtcatctctttctacaatgtgaccaacaagtcccacatctttactttcacccacaatttctctggcccccttcgccctttctttgaaccttgccttcatgatggaggaaaaaacacagcacctctagtcatttgttcagaactacacaaatcagaggaatcaattgtccccaggccagaagggaaaggccatgctaatggagatgtgtccctcaaggtgaactcttctttactacccccgaaggccccagagctgaaggatataatcctgtccttgccccctgaccttggcccagcccttcaggagctcaaggctccttctttttagggatatgccacattacctgctcccatcaccatccagcccagcaccctggacttcagtcgcctggcccaaccccatgattatggaacgtctcttcaccttaacccaaatccagacccttttgtggtttctatttgt

SEQ ID NO: 42 - DNA, 3381 bp, Homo sapiens
ctcatatttcataaaatatgaacttttcccggcccacatccctaggccttcctgatgcgcttgcctgctccctggtctctctgcatggggaaggagtgttcccagcttgcaaactccagctttgcctgtgagaggaacaagcgtccctgatccagaaggtgttcagatggagatggcgagttctgctggctcctggctctctggctgcctcatccctctcgtcttcctccggctgtctgtgcatgtgtcaggccacgcaggggatgccggcaagttccacgtggccctactagggggcacagccgagctgctctgccctctctccctctggcccgggacggtacccaaggaggtgaggtggctgcggtccccattcccgcagcgttcccaggctgttcacatattccgggatgggaaggaccaggatgaagatctgatgccggaatataaggggaggacggtgctagtgagagatgcccaagagggaagtgtcactctgcagatccttgacgtgcgccttgaggaccaagggtcttaccgatgtctgatccaagttggaaatctgagtaaagaggacaccgtgatcctgcaggttgcagccccatctgtggggagtctctccccctcagcagtggctctggctgtgatcctgcctgtcctggtacttctcatcatggtgtgcctttgccttatctggaagcaaagaagagcaaaagaaaagcttctctatgaacatgtgacggaggtggacaatcttctttcagaccatgctaaagaaaaaggaaaactccataaagctgtcaagaaactccggagtgaactgaagttgaaaagagctgcagcaaactcaggctggagaagagcccggttgcattttgtggcagtgaccctggacccagacacagcacatcccaaactcatcctttctgaggaccaaagatgtgtaaggcttggagacagacggcagcctgtacctgacaacccccagagatttgatttcgttgtcagcatcctaggctctgagtacttcacgactggctgccactactgggaggtgtatgtgggagacaagaccaaatggattcttggagtatgtagtgagtcagtgagcaggaaggggaaggttactgcctcacctgccaatggacactggcttctgcgacagagtcgtgggaatgagtatgaagctctcacatccccgcagacctccttccgccttaaagagcctccacggtgtgtggggattttcctggactatgaagcaggagtcatctctttctacaatgtgaccaacaagtcccacatctttactttcacccacaatttctctggcccccttcgccctttctttgaaccttgccttcatgatggaggaaaaaacacagcacctctagtcatttgttcagaactacacaaatcagaggaatcaattgtccccaggccagaagggaaaggccatgctaatggagatgtgtccctcaaggtgaactcttctttactacccccgaaggccccagagctgaaggatataatcctgtccttgccccctgaccttggcccagcccttcaggagctcaaggctccttctttttagggatatgccacattacctgctcccatcaccatccagcccagcaccctggacttcagtcgcctggcccaaccccatgattatggaacgtctcttcaccttaacccaaatccagacccttttgtggtttctatttgtaccacttttctcccaggcctcagttctgaagcttacctttcttctaaggaattgaagctcccagtgacctggagggaggattcctggaaaccaaacaatcagtttaggtgcaggtggagatgttgaatatgtgttaccaagatacagcacaggttcagggaaaagagttcgctactccaggggttatttagaagacactttctctgcctcatcctgccctcaagctttagtcaagaagttatggcccccagtccctgacttcttacttatcccattgaggactgcctttctctctctcagttctggcctctgccccccaaagtcagctctctaaaagcaagcatgttttagaccactcactctttccctctttcttcaggaatgaattgggaaaggctgatgagtaaaacataccatccttttctattttcttgatgctgtttacaacatagtttggtgatatccagagctaatgtacatgctttcaaaagctaatctgcctgttgatgataactaggtacagcgactttaaatacagttgctataatcctgaaaagccccaggagcacatcagggagctgggaaacacagttgcagagaactcagctgtatgttgccctctgaccttggcccagaccttcagaggctcagtctgttgagtctgttgtgactgtcttatgaatcaatctgttgtgccaaccctttctgagattcagagaggtccagctagaaaaagtggcagttttaaccaccaacgtagaagcttttttttccccacaagaccattgtactgcaatacttgactatttgtgtagcaaacagcctcttaggttggagagtattgatttcaaataggaagttggtagactaggtgtgaggatgaaataatgaccttgatttttgggtgtgtattgcagaagcctcgtcgctttcaagtcacatcatatatgcgatctggcctaaccatggagatattggttatcagtttgcagatgaatacacagttctatccaacagaaactgtatctttctgtttgtcagaagttctccgttagttcctgtattagtcaaggttttccagagaaacagaatcaatatatattggggcaggggggcggtggttattataaggaattggctcacgtgattatggaggcggttaaatcccaagatctgcaggataagtcagcaagatggagtcccatgagagctgatggtttagttccagtctgatggcagcaggcttgacgccaaggaagagatgatgtttaattcaagtccgaaggcaaggaaaaagctgatggtcctgtccaaaggctattaggcaggaagaattctcttagggcagagttagctcttttgttctattcaggccttcaactgattcagcaaggcccgcccacatttgggaggacagtctgcattactcagtctactgatttgaatgttaatgtcattgagaaacaccctcacaggaacactcagaataatgtttgaccaaatagctgggcatcttgtgacccagttaagtggatacataagattaactatcacagtgactcagtggaattttttgtttgcttttgtacagttttaaaataaaagtttggtatttgtgttttaaaaaaaaaaaaaaaaaaaaaaaaaaaaaa

SEQ ID NO: 43 - protein, 184 aa, Homo sapiens
MPFNGEKQCVGEDQPSDSDSSRFSESMASLSDYECSRQSFTSDSSSKSSSPASTSPPRVVTFDEVMATARNLSNLTLAHEIAVNENLQLKQEALPEKSLAGRVKHIVHQAFWDVLDSELNADPPEIEHAIKLFEEIREILLSFLTPGGNRLRNQICEVLDTDLIRQQAEHSAVDIQGLANYVIS

SEQ ID NO: 44 - protein, 258 aa, Homo sapiens
MPFNGEKQCVGEDQPSDSDSSRFSESMASLSDYECSRQSFTSDSSSKSSSPASTSPPRVVTFDEVMATARNLSNLTLAHEIAVNENFQLKQEALPEKSLAGRVKHIVHQAFWDVLDSELNADPPEFEHAIKLFEEIREILLSFLTPGGNRLRNQICEVLDTDLIRQQAEHSAVDIQGLANYVISTMGKLCAPVRDNDIRELKATGNIVEVLRQIFHVLDLMQMDMANFTIMSLRPHLQRQLVEYERTKFQEILEETPS

SEQ ID NO: 45 - protein, 17 aa, Homo sapiens
MPFNGEKQCVGEDQPSD

SEQ ID NO: 46 - protein, 17 aa, Homo sapiens
MPFNGEKQCVGEDQPSD

SEQ ID NO: 47 - protein, 122 aa, Homo sapiens
ARDYLKTLTERLARLRRARRALRRRNSIKKMAALTPRKRKQDSLKCDSLLHFTENLFPSPNKKHCFYQNSDKNEENLHCSQQEHFVLSALKTTEINRLPSANQGSPFKSALSTVSFYNQNKW

SEQ ID NO: 48 - protein, 297 aa, Homo sapiens
RRNSIKKMAALTPRKRKQDSLKCDSLLHFTENLFPSPNKKHCFYQNSDKNEENLHCSQQEHFVLSALKTTEINRLPSANQGSPFKSALSTVSFYNQNKWYLNPLERKLIKESRSTCLKTNDEDKSFPIVTEKMQGKPVCSKKNNKKPQKSLTAKYQPKYRHIKPVSRNSRNSKQNRVIYKPIVEKENNCHSAENNSNAPRVLSQKIKPQVTLQGGAAFFVRKKSSLRKSSLENEPSLGRTQKSKSEVIEDSDVETVSEKKTFATRQVPKCLVLEEKLKIGLLSASSKNKEKLIKVKL

SEQ ID NO: 49 - protein, 60 aa, Homo sapiens
SLLHFTENLFPSPNKKHCFYQNSDKNEENLHCSQQEHFVLSALKTTEINRLPSANQGSPF

SEQ ID NO: 50 - protein, 59 aa, Homo sapiens
GARILETATRVGGARAGAPPITEPAPGSAEPWPIGTQRLPPSLARDYLKTLTERLARLR

SEQ ID NO: 51 - protein, 23 aa, Homo sapiens
RNSIKKMAALTPRKRKQDSLKCD

SEQ ID NO: 52 - protein, 156 aa, Homo sapiens
GARILETATRVGGARAGAPPITEPAPGSAEPWPIGTQRLPPSLARDYLKTLTERLARLRRARRALRRRNSIKKMAALTPRKRKQDSLKCDSLLHFTENLFPSPNKKHCFYQNSDKNEENPHCSQQEHFVLSALKTTEINRLPSANQGSPFKSALST

SEQ ID NO: 53 - protein, 313 aa, Homo sapiens
LKTLTERLARLRRARRALRRRNSIKKMAALTPRKRKQDSLKCDSLLHFTENLFPSPNKKHCFYQNSDKNEENLHCSQQEHFVLSALKTTEINRLPSANQGSPFKSALSTVSFYNQNKWYLNPLERKLIKESRSTCLKTNDEDKSFPIVTEKMQGKPVCSKKNNKKPQKSLTAKYQPKYRHIKPVSRNSRNSKQNRVIYKPIVEKENNCHSAENNSNAPRVLSQKIKPQVTLQGGAAFFVRKKSSLRKSSLENEPSLGRTQKSKSEVIEDSDVETVSEKKTFATRQVPKCLVLEEKLKIGLLSASSKNKEKLIK

SEQ ID NO: 54 - protein, 279 aa, Homo sapiens
RKKVNPYEEVDQEKYSNLVQSVLSSRGVAQTPGSVEEDALLCGPVSKHKLPNQGEDRRVPQNWFPIFNPERSDKPNASDPSVPLKIPLQRNVIPSVTRVLQQTMAKQQVFLLERWKQRMILELGEDGFKEYTSNVFLQGKRFHEALESILSPQETLKERDENLLKSGYIESVQHILKDVSGVRALESAVQHETLNYIGLLDCVAEYQGKLCVIDWKTSEKPKPFIQSIFDNPLQVVAYMGAMNHDTNYSFQVQCGLIVVAYKDGSPAHPHFMDAELCSQ

SEQ ID NO: 55 - protein, 307 aa, Homo sapiens
RKKVNPYEEVDQEKYSNLVQSVLSSRGVAQTPGSVEEDALLCGPVSKHKLPNQGEDRRVPQNWFPIFNPERSDKPNASDPSVPLKIPLQRNVIPSVTRVLQQTMTKQQVFLLERWKQRMILELGEDGFKEYTSNVFLQGKRFHEALESILSPQETLKERDENLLKSGYIESVQHILKDVSGVRALESAVQHETLNYIGLLDCVAEYQGKLCVIDWKTSEKPKPFIQSTFDNPLQVVAYMGAMNHDTNYSFQVQCGLIVVAYKDGSPAHPHFMDAELCSQYWTKWLLRLEEYTEKKKNQNIQKPEYSE

SEQ ID NO: 56 - protein, 82 aa, Homo sapiens
MKLFQTICRQLRSSKFSVESAALVAFSTSSYSCGRKKKVNPYEEVDQEKYSNLVQSVLSSRGVAQTPGSVEEDALLCGPVSK

SEQ ID NO: 57 - protein, 298 aa, Homo sapiens
QHEEFILLSQGEVEKLIKCDEIQVDSEEPVFEAVINWVKHAKKEREESLPNLLQYVRMPLLTPRYITDVIDAEPFIRCSLQCRDLVDEAKKFHLRPELRSQMQGPRTRARLGANEVLLVVGGFGSQQSPIDVVEKYDPKTQEWSFLPSITRKRRYVASVSLHDRIYVIGGYDGRSRLSSVECLDYTADEDGVWYSVAPMNVRRGLAGATTLGDMIYVSGGFDGSRRHTSMERYDPNIDQWSMLGDMQTAREGAGLVVASGVIYCLGGYDGLNILNSVEKYDPHTGHWTNVTPMATKRS

SEQ ID NO: 58 - protein, 189 aa, Homo sapiens
VDSEEPVFEAVINWVKHAKKEREESLPNLLQYVRMPLLTPRYITDVIDAEPFIRCSLQCRDLVDEAKKFHLRPELRSQMQGPRTRARLGANEVLLVVGGFGSQQSPIDVVEKYDPKTQEWSFLPSITRKRRYVASVSLHDRIYVIGGYDGRSRLSSVECLDYTADEDGVWYSVAPMNVRRGLAGATTLG

SEQ ID NO: 59 - protein, 448 aa, Homo sapiens
QLKGVKQACCEFLESQLDPSNCLGIRDFAETHNCVDLMQAAEVFSQKHFPEVVQHEEFILLSQGEVEKLIKCDEIQVDSEEPVFEAVINWVKHAKKEREESLPNLLQYVRMPLLTPRYITDVIDAEPFIRCSLQCRDLVDEAKKFHLRPELRSQMQGPRTRARLGANEVLLVVGGFGSQQSPIDVVEKYDPKTQEWSFLPSITRKRRYVASMSLHDRIYVIGGYDGRSRLSSVECLDYTADEDGVWYSVAPMNVRRGLAGATTLGDMIYVSGGFDGSRRHTSMERYDPNIDQWSMLGDMQTAREGAGLVVASGVIYCLGGYDGLNILNSVEKYDPHTGHWTNVTPMATKRSGAGVALLNDHIYVVGGFDGTAHLSSVEAYNIRTDSWTTVTSMTTPRCYVGATVLRGRLYAIAGYDGNSLLSSIECYDPIIDSWEVVTSMGTQRCDAG

SEQ ID NO: 60 - protein, 189 aa, Homo sapiens
MIYVSGGFDGSRRHTSMERYDPNIDQWSMLGDMQTAREGAGLVVASGVIYCLGGYDGLNILNSVEKYDPHTGHWTNVTPMATKRSGAGVALLNDHIYVVGGFDGTAHLSSVEAYNIRTDSWTTVTSMTTPRCYVGATVLRGRLYAIAGYDGNSLLSSIECYDPIIDSWEVVTSMGTQRCDAGVCVLREK

SEQ ID NO: 61 - protein, 189 aa, Homo sapiens
VDSEEPVFEAVINWVKHAKKEREESLPNLLQYVRMPLLTPRYITDVIDAEPFIRCSLQCRDLVDEAKKFHLRPELRSQMQGPRTRARLGANEVLLVVGGFGSQQSPIDVVEKYDPKTQEWSFLPSITRKRRYVASMSLHDRIYVIGGYDGRSRLSSVECLDYTADEDGVWYSVAPMNVRRGLAGATTLG

SEQ ID NO: 62 - protein, 414 aa, Homo sapiens
MGGIMAPKDIMTNTHAKSILNSMNSLRKSNTLCDVTLRVEQKDFPAHRIVLAACSDYFCAMFTSELSEKGKPYVDIQGLTASTMEILLDFVYTETVHVTVENVQELLPAACLLQLKGVKQACCEFLESQLDPSNCLGIRDFAETHNCVDLMQAAEVFSQKHFPEVVQHEEFILLSQGEVEKLIKCDEIQVDSEEPVFEAVINWVKHAKKEREESLPNLLQYVRMPLLTPRYITDVIDAEPFIRCSLQCRDLVDEAKKFHLRPELRSQMQGPRTRARLDMIYVSGGFDGSRRHTSMERYDPNIDQWSMLGDMQTAREGAGLVVASGVIYCLGGYDGLNILNSVEKYDPHTGHWTNVTPMATKRSGAGVALLNDHIYVVGGFDGTAHLSSVEAYNIRTDSWTTVTSMTTPRCYVGA

SEQ ID NO: 63 - protein, 164 aa, Homo sapiens
RKSNTLCDVTLRVEQKDFPAHRIVLAACSDYFCAMFTSELSEKGKPYVDIQGLTASTMEILLDFVYTETVHVTVENVQELLPAACLLQLKGVKQACCEFLESQLDPSNCLGIRDFAETHNCVDLMQAAEVFSQKHFPEVVQHEEFILLSQGEVEKLIKCDEIQV

SEQ ID NO: 64 - protein, 299 aa, Homo sapiens
TGDFRYTPSMLKEPALTLGKQIHTLYLDNTNCNPALVLPSRQEAAHQIVQLIRKHPQHNIKIGLYSLGKESLLEQLALEFQTWVVLSPRRLELVQLLGLADVFTVEEKAGRIHAVDHMEICHSNMLRWNQTHPTIAILPTSRKIHSSHPDIHVIPYSDHSSYSELRAFVAALKPCQVVPIVSRRPCGGFQDSLSPRISVPLIPDSVQQYMSSSSRKPSLLWLLERRLKRPRTQGVVFESPEESADQSQADRDSKKAKKEKLSPWPADLEKQPSHHPLRIKKQLFPDLYSKEWNKAVPFC

SEQ ID NO: 65 - protein, 64 aa, Homo sapiens
MNGVLIPHTPIAVDFWSLRRAGTARLFFLSHMHSDHTVGLSSTWARPLYCSPITAHLLHRHLQV

SEQ ID NO: 66 - protein, 19 aa, Homo sapiens
AGYSSRRFDQQVEKYHKPC

SEQ ID NO: 67 - protein, 421 aa, Homo sapiens
FGTILYTGDFRYTPSMLKEPALTLGKQIHTLYLDNTNCNPALVLPSRQEAAHQIVQLIRKHPQHNIKIGLYSLGKESLLEQLALEFQTWVVLSPRRLELVQLLGLADVFTVEEKAGRIHAVDHMEICHSNMLRWNQTHPTIAILPTSRKIHSSHPDIHVIPYSDHSSYSELRAFVAALKPCQVVPIVSRRPCGGFQDSLSPRISVPLIPDSVQQYMSSSSRKPSLLWLLERRLKRPRTQGVVFESPEESADQSQADRDSKKAKKEKLSPWPADLEKQPSHHPLRIKKQLFPDLYSKEWNKAVPFCESQKRVTMLTAPLGFSVHLRSTDEEFISQKTREEIGLGSPLVPMGDDDGGPEATGNQSAWMGHGSPLSHSSKGTPLLATEFRGLALKYLLTPVNFFQAGYSSRRFDQQVEKYHKPC

SEQ ID NO: 68 - protein, 307 aa, Homo sapiens
MNGVLIPHTPIAVDFWSLRRAGTARLFFLSHMHSDHTVGLSSTWARPLYCSPITAHLLHRHLQVSKQWIQALEVGESHVLPLDEIGQETMTVTLLDANHCPGSVMFLFEGYFGTILYTGDFRYTPSMLKEPALTLGKQIHTLYLDNTNCNPALVLPSRQEAAHQIVQLIRKHPQHNIKIGLYSLGKESLLEQLALEFQTWVVLSPRRLELVQLLGLADVFTVEEKAGRIHAVDHMEICHSNMLRWNQTHPTIAILPTSRKIHSSHPDIHVIPYSDHSSYSELRAFVAALKPCQVVPIVSRRPWEAFR

SEQ ID NO: 69 - protein, 336 aa, Homo sapiens
MATALSEEELDNEDYYSLLNVRREASSEELKAAYRRLCMLYHPDKHRDPELKSQAERLFNLVHQAYEVLSDPQTRAIYDIYGKGGLEMEGWEVVERRRTPAEIREEFERLQREREERRLQQRTNPKGTISVGVNATDLFDRYDEEYEDVSGSSFPQIEINKMHISQSIEAPLTATDTAILSGSLSTQNGNGGGSINFALRRVTSVKGWGELEFGAGDLQGPLFGLKLFRNLTPRCFVTTNCALQFSSRGIRPGLTTVLARNLDKNTVGYLQWRWGIQSAMNTSIVRDTKTSHFTVALQLGIPHSFALISYQHKFQDDDQTRVKGSLKAGFFGTVVE

SEQ ID NO: 70 - protein, 559 aa, Homo sapiens
MATALSEEELDNEDYYSLLNVRREASSEELKAAYRRLCMLYHPDKHRDPELKSQAERLFNLVHQAYEVLSDPQTRAIYDIYGKRGLEMEGWEVVERRRTPAEIREEFERLQREREERRLQQRTNPKGTISVGVDATDLFDRYDEEYEDVSGSSFPQIEINKMHISQSIEAPLTATDTAILSGSLSTQNGNGGGSINFALRRVTSAKGWGELEFGAGDLQGPLFGLKLFRNLTPRCFVTTNCALQFSSRGIRPGLTTVLARNLDKNTVGYLQWRWGIQSAMNTSIVRDTKTSHFTVALQLGIPHSFALISYQHKFQDDDQTRVKGSLKAGFFGTVVEYGAERKISRHSVLGAAVSVGVPQGVSLKVKLNRASQTYFFPIHLTDQLLPSAMFYATVGPLVVYFAMHRLIIKPYLRAQKEKELEKQRESAATDVLQKKQEAESAVRLMQESVRRIIEAEESRMGLIIVNAWYGKFVNDKSRKSEKVKVIDVTVPLQCLVKDSKLILTEASKAGLPGFYDPCVGEEKNLKVLYQFRGVLHQVMVLDSEALRIPKQSHRIDTDG

SEQ ID NO: 71 - protein, 103 aa, Homo sapiens
DSVSKKKKKKEIHKVVERRRTPAEIREEFERLQREREERRLQQRTNPKGTISVGVDATDLFDRYDEEYEDVSGSSFPQIEINKMHISQSIEAPLTATDTAILS

SEQ ID NO: 72 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 73 - protein, 173 aa, Homo sapiens
TRAHQESAEPKYLPHKTCNEIIVPKAPSHKTIQETPHSEDYSIEINQETPGSEKYSPETYQEIPGLEEYSPEIYQETSQLEEYSPEIYQETPGPEDLSTETYKNKDVPKECFPEPHQETGGPQGQDPKAHQEDAKDAYTFPQEMKEKPKEEPGIPAILNESHPENDVYSYVLF

SEQ ID NO: 74 - protein, 484 aa, Homo sapiens
MDLGKDQSHLKHHQTPDPHQEENHSPEVIGTWSLRNRELLRKRKAEVHEKETSQWLFGEQKKRKQQRTGKGNRRGRKRQQNTELKVEPQPQIEKEIVEKALAPIEKKTEPPGSITKVFPSVASPQKVVPEEHFSEICQESNIYQENFSEYQEIAVQNHSSETCQHVSEPEDLSPKMYQEISVLQDNSSKICQDMKEPEDNSPNTCQVISVIQDHPFKMYQDMAKREDLAPKMCQEAAVPKILPCPTSEDTADLAGCSLQAYPKPDVPKGYILDTDQNPAEPEEYNETDQGIAETEGLFPKIQEIAEPKDLSTKTHQESAEPKYLPHKTCNEIIVPKAPSHKTIQETPHSEDYSIEINQETPGSEKYSPETYQEIPGLEEYSPEIYQETSQLEEYSPEIYQETPGPEDLSTETYKNKDVPKECFPEPHQETGGPQGQDPKAHQEDAKDAYTFPQEMKEKPKEEPGIPAILNESHPENDVYSYVLF

SEQ ID NO: 75 - protein, 484 aa, Homo sapiens; identical to SEQ ID NO: 74

SEQ ID NO: 76 - protein, 331 aa, Homo sapiens
MWLWEDQGGLLGPFSFLLLVLLLVTRSPVNACLLTGSLFVLLRVFSFEPVPSCRALQVLKPRDRISAIAHRGGSHDAPENTLAAIRQAAKNGATGVELDIEFTSDGIPVLMHDNTVDRTTDGTGRLCDLTFEQIRKLNPAANHRLRNDFPDEKIPTLREAVAECLNHNLTIFFDVKGHAHKATEALKKMYMEFPQLYNNSVVCSFLPEVIYKMRQTDRDVITALTHRPWSLSHTGDGKPRYDTFWKHFIFVMMDILLDWSMHNILWYLCGISAFLMQKDFVSPAYLKKWSAKGIQVVGWTVNTFDEKSYYESHLGSSYITDSMVEDCEPHF

SEQ ID NO: 77 - protein, 188 aa, Homo sapiens
FTTGCHYWEVYVGDKTKWILGVCSESVSRKGKVTASPANGHWLLRQSRGNEYEALTSPQTSFRLKEPPRCVGIFLDYEAGVISFYNVTNKSHIFTFTHNFSGPLRPFFEPCLHDGGKNTAPLVICSELHKSEESIVPRPEGKGHANGDVSLKVNSSLLPPKAPELKDIILSLPPDLGPALQELKAPSF

SEQ ID NO: 78 - protein, 475 aa, Homo sapiens
MEMASSAGSWLSGCLIPLVFLRLSVHVSGHAGDAGKFHVALLGGTAELLCPLSLWPGTVPKEVRWLRSPFPQRSQAVHIFRDGKDQDEDLMPEYKGRTVLVRDAQEGSVTLQILDVRLEDQGSYRCLIQVGNLSKEDTVILQVAAPSVGSLSPSAVALAVILPVLVLLIMVCLCLIWKQRRAKEKLLYEHVTEVDNLLSDHAKEKGKLHKAVKKLRSELKLKRAAANSGWRRARLHFVAVTLDPDTAHPKLILSEDQRCVRLGDRRQPVPDNPQRFDFVVSILGSEYFTTGCHYWEVYVGDKTKWILGVCSESVSRKGKVTASPANGHWLLRQSRGNEYEALTSPQTSFRLKEPPRCVGIFLDYEAGVISFYNVTNKSHIFTFTHNFSGPLRPFFEPCLHDGGKNTAPLVICSELHKSEESIVPRPEGKGHANGDVSLKVNSSLLPPKAPELKDIILSLPPDLGPALQELKAPSF